(19) United States
(12) Patent Application Publication
(10) Pub. No.: US 2024/0246944 A1
(43) Pub. Date: Jul. 25, 2024

(54) AMINO HETEROARYL COMPOUNDS AND COMPOSITIONS

(71) Applicant: Anrui Biomedical Technology (Guangzhou) Co., Ltd., Guangzhou, Guangdong (CN)

(72) Inventors: Dai Cheng, Guangzhou (CN); Qiang Ding, Guangzhou (CN)

(21) Appl. No.: 18/550,898

(22) PCT Filed: Jul. 13, 2021
(86) PCT No.: PCT/CN2021/106028

§ 371 Date: Sep. 15, 2023

(30) Foreign Application Priority Data
Mar. 16, 2021 (WO) PCT/CN2021/081177

(51) Int. Cl. C07D 413/12; A61K 31/4439; A61K 31/444; A61K 31/501; A61K 31/506; A61K 31/551; A61K 31/553; A61P 17/06; C07D 401/04; C07D 401/12; C07D 403/12; C07D 471/10; C07D 487/04

(52) U.S. Cl. CPC C07D 413/12; A61K 31/4439; A61K 31/444; A61K 31/501; A61K 31/506; A61K 31/551; A61K 31/553; A61P 17/06; C07D 401/04; C07D 401/12; C07D 403/12; C07D 471/10; C07D 487/04

(57) ABSTRACT

Provided herein are novel compounds (e.g., Formula I or II), pharmaceutical compositions, and methods of using related to Tyrosine kinase 2 (TYK2). The compounds herein are typically TYK2 inhibitors, which can be used for treating a variety of diseases or disorders, such as an autoimmune disorder or an inflammatory disorder, e.g., psoriasis, psoriatic arthritis, Crohn's disease, ulcerative colitis, inflammatory bowel disease, and/or systemic lupus erythematosus.

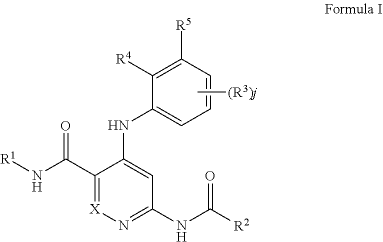

This application claims priority of International Application No. PCT/CN2021/081177, filed Mar. 16, 2021, the content of which is incorporated herein by reference in its entirety.

In various embodiments, the present disclosure generally relates to novel heteroaryl compounds, compositions comprising the same, methods of preparing and methods of using the same, e.g., for inhibiting tyrosine-protein kinase 2 (TYK2) and/or for treating or preventing various diseases or disorders described herein.

### BACKGROUND

Tyrosine kinase 2 (TYK2) is a member of the Janus kinase (JAK) family of nonreceptor tyrosine kinases. It has been shown that TYK2 is critical in regulating the signal transduction cascade downstream of receptors for IL-12, IL-23 and type I interferons (e.g., IFN-alpha or IFN-beta) both in mice and in human. TYK2 mediates the receptor-induced phosphorylation of members of the Signal Transducer and Activation of Transcription (STAT) family of transcription factors, an essential signal that leads to the dimerization of STAT proteins and the transcription of STAT-dependent pro-inflammatory genes.

Various diseases or disorders such as autoimmune diseases, inflammatory diseases, etc. are known to be associated/mediated by TYK2. Clinically, a TYK2 inhibitor, BMS-986165, is currently in phase III trial for treating psoriasis. BMS-986165 is also in various trials for treating other diseases such as Crohn's disease, psoriatic arthritis, systemic lupus erythematosus, ulcerative colitis, and inflammatory bowel disease. New TYK2 inhibitors are needed to provide therapeutic benefits to a wide variety of patients in need thereof.

### BRIEF SUMMARY

In various embodiments, the present disclosure is based in part on the newly designed heteroaryl compounds as TYK2 inhibitors, which can bind to the pseudokinase domain or JH2 domain. The compounds and compositions herein are useful for treating various diseases or disorders, such as an autoimmune disorder or an inflammatory disorder, e.g., psoriasis, psoriatic arthritis, Crohn's disease, ulcerative colitis, inflammatory bowel disease, and/or systemic lupus erythematosus.

Some embodiments of the present disclosure are directed to a compound of Formula I or II, or a pharmaceutically acceptable salt thereof,

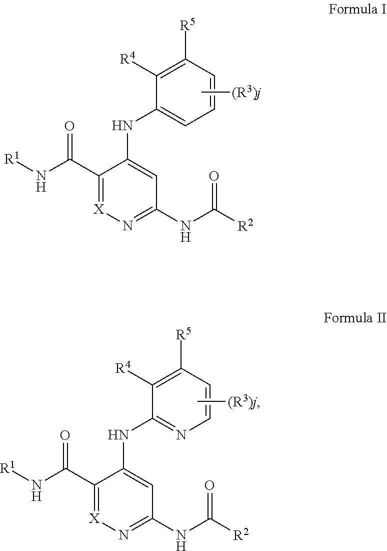

wherein X, R¹, R², R³, j, R⁴, and R⁵ are defined herein for the respective formulae. In some embodiments, the compound of Formula I can have a sub-formula of I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3 as defined herein. In some embodiments, the present disclosure also provides specific compounds selected from the compounds shown in Table 1 herein, or a pharmaceutically acceptable salt thereof.

In some embodiments, the present disclosure provides a pharmaceutical composition comprising one or more compounds of the present disclosure and optionally a pharmaceutically acceptable excipient. The pharmaceutical composition can be typically formulated for oral administration.

In some embodiments, the present disclosure also provides a method of inhibiting TYK2 in a subject or biological sample. In some embodiments, the method comprises contacting the subject or biological sample with an effective amount of one or more compounds of the present disclosure, e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof, or a pharmaceutical composition comprising the same.

In some embodiments, the present disclosure provides a method of treating or preventing a TYK2-mediated disease or disorder in a subject in need thereof. In some embodiments, the method comprises administering to the subject an effective amount of one or more compounds of the present disclosure or the pharmaceutical composition herein. In some embodiments, the method comprises administering to the subject an effective amount of a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof, or a pharmaceutical composition comprising the same. In some embodiments, the TYK2-mediated disease or disorder is an autoimmune disease or disorder, an inflammatory disease or disorder, a proliferative disease or disorder, an endocrine disease or disorder, a neurological disease or disorder, and/or a disease or disorder associated with transplantation. In some embodiments, the TYK2 mediate disease or disorder is psoriasis, psoriatic arthritis, Crohn's disease, ulcerative colitis, inflammatory bowel disease, and/or systemic lupus erythematosus. In some embodiments, the administering is an oral administration. In some embodiments, the method herein further comprises administering to the subject an additional therapeutic agent.

It is to be understood that both the foregoing summary and the following detailed description are exemplary and explanatory only, and are not restrictive of the invention herein.

### BRIEF DESCRIPTION OF THE DRAWINGS

FIG. 1 is a bar graph showing the effects of reducing interferon gamma production induced by IL-12/IL-18 in mice following oral administration of compound Example 4 at 2.5 mpk, 5 mpk, 7.5 mpk, or 10 mpk. BMS986165 at 5 mpk was used as a positive control.

FIG. 2 is a bar graph showing the effects of reducing interferon gamma production induced by IL-12/IL-18 in mice following oral administration of compound Example 5 at 2.5 mpk, 5 mpk, or 10 mpk. BMS986165 at 5 mpk was used as a positive control.

### DETAILED DESCRIPTION

In various embodiments, the present disclosure provides compounds and compositions that are useful for inhibiting TYK2 and/or treating or preventing various diseases or disorders described herein. In some embodiments, the present disclosure is directed to the various objects as follows. For example, one object of the present disclosure is to provide a TYK2 inhibitor, such as those with similar potency when compared to BMS986165 when tested with the in vitro and/or in vivo methods described herein. One object of the present disclosure is to provide a selective TYK2 inhibitor over other JAK kinases, such as similar or more selective TYK2 inhibitors when compared to BMS986165 when tested with the methods described herein. One object of the present disclosure is to provide an orally bioavailable TYK2 inhibitor, such as those with similar or better pharmacokinetic profiles when compared to BMS986165 in mouse, rat, dog, or other animal species or in human. One object of the present disclosure includes providing a synthetic method for the TYK2 inhibitors described herein, a pharmaceutical composition comprising the TYK2 inhibitors described herein, a use of the TYK2 inhibitors described herein or compositions thereof for treating or preventing various diseases or disorders described herein, etc. In some embodiments, the TYK2 inhibitor is a compound of Formula I or II as defined herein. The various embodiments described herein can achieve one or more of these non-limiting objects of the present disclosure.

### Compounds

In some embodiments, the present disclosure provides a compound of Formula I, or a pharmaceutically acceptable salt thereof:

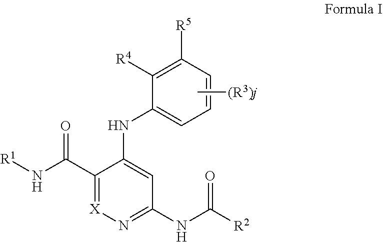

wherein:
X is CH or N;
R¹ is C₁₋₃ alkyl substituted by 0-7 deuterium atoms;
R² is optionally substituted C₁₋₆ alkyl, optionally substituted C₁₋₄ heteroalkyl, optionally substituted cycloalkyl (e.g., C₃₋₆ cycloalkyl), optionally substituted heterocyclyl (e.g., 4-8 membered heterocyclyl), or optionally substituted heteroaryl;
R³ at each occurrence is independently halogen, optionally substituted C₁₋₄ alkyl, or optionally substituted C₁₋₄ heteroalkyl;
j is 0, 1, 2, or 3;
R⁴ is C₁₋₆ alkyl optionally substituted with 1-3 R^{A}, S(O)ₚR^{B}, or OR^{C};
   wherein:
   p is 0, 1, or 2,
   R^{A} at each occurrence is independently halogen, OH, C₁₋₆ alkyl optionally substituted with 1-3 R^{A1},
   R^{B} is C₁₋₆ alkyl optionally substituted with 1-3 R^{A1},
   R^{C} is hydrogen or C₁₋₆ alkyl optionally substituted with 1-3 R^{A2},
   wherein R^{A1} at each occurrence is independently halogen, OH, or CN; R^{A2} at each occurrence is independently F or OH;
R⁵ is an optionally substituted heterocyclyl or an optionally substituted heteroaryl such as

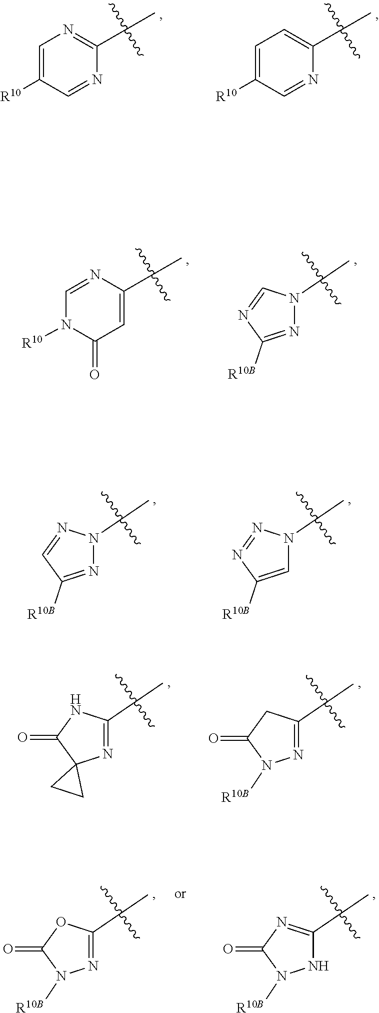

or R⁵ is -L¹-L²-Q-G, wherein:
R¹⁰ at each occurrence is independently an optionally substituted cycloalkyl (e.g., C₃₋₆ cycloalkyl), or optionally substituted heterocyclyl (e.g., 4-8 membered heterocyclyl); R^{10B} at each occurrence is independently halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl);
L¹ is O, C═(O)NH, or null,
L² is C₁₋₄ alkylene, or null,
Q is an optionally substituted heterocycle or optionally substituted heteroaryl, and
G is CN,

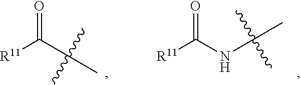

or a Michael acceptor,
wherein R¹¹ is hydrogen, an optionally substituted C₁₋₆ alkyl or an optionally substituted C₃₋₆ cycloalkyl.

In some embodiments, X in Formula I can be CH, and the compound of Formula I can be characterized as having a Formula I-1:

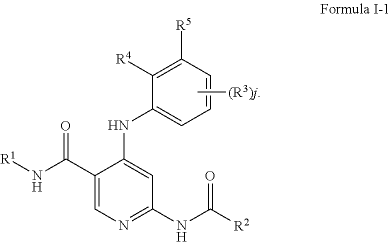

In some embodiments, X in Formula I can be N, and the compound of Formula I can be characterized as having a Formula I-2:

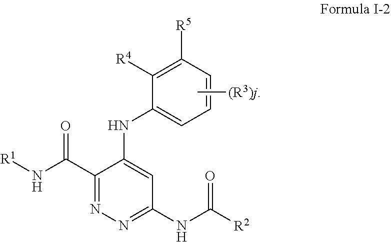

R¹ in Formula I (e.g., Formula I-1 or I-2) is typically a methyl or ethyl group, optionally substituted with deuterium atoms. For example, in some embodiments, R¹ is CH₃, C₂H₅, CD₃, or CD₂CD₃. In some preferred embodiments, R¹ is CD₃.

Various groups can be suitable for R². In some embodiments, R² in Formula I (e.g., Formula I-1 or I-2) can be an optionally substituted C₁₋₆ alkyl, such as optionally substituted methyl, ethyl, or isopropyl. In some embodiments, R² in Formula I (e.g., Formula I-1 or I-2) can be an optionally substituted C₁₋₄ heteroalkyl. In some embodiments, R² in Formula I (e.g., Formula I-1 or I-2) can be an optionally substituted cycloalkyl (e.g., C₃₋₆ cycloalkyl). The cycloalkyl can be typically a monocyclic or a bicyclic ring (including a fused, spiro, or bridged bicyclic) having 3-8 ring carbons, more typically, 3-6 ring carbons. In some embodiments, R² in Formula I (e.g., Formula I-1 or I-2) can be an optionally substituted heterocyclyl (e.g., 4-8 membered heterocyclyl). The heterocyclyl is typically a monocyclic or a bicyclic ring (including a fused, spiro, or bridged bicyclic) having 4-10 ring atoms, with 1-3 ring heteroatoms independently selected from N, O, and S. More typically, the heterocyclyl can have 4-8 ring atoms with 1 or 2 ring heteroatoms independently selected from N, O, and S. In some embodiments, R² in Formula I (e.g., Formula I-1 or I-2) can be an optionally substituted heteroaryl. The heteroaryl is typically a 5- or 6-membered heteroaryl having 1-4 ring heteroatoms independently selected from N, O, and S. When substituted, the C₁₋₆ alkyl, cycloalkyl, heterocyclyl, or heteroaryl can be substituted with one or more, typically, 1-5, independently selected substituents, which include any of those described herein.

In some particular embodiments, R² in Formula I (e.g., Formula I-1 or I-2) can be a 3-6 membered cycloalkyl, such as cyclopropyl, cyclobutyl, which is optionally substituted. For example, in some embodiments, R² in Formula I (e.g., Formula I-1 or I-2) can be a C₃₋₆ cycloalkyl, which can be substituted with 1-4 (e.g., 1, 2, or 3) substituents independently selected from CN, halogen (e.g., F), OH, and optionally substituted C₁₋₆ alkyl. In some embodiments, R² in Formula I (e.g., Formula I-1 or I-2) can be a C₃₋₆ cycloalkyl, which can be optionally substituted with 1-4 (e.g., 1, 2, or 3) substituents independently selected from CN, F, Cl, OH, and C₁₋₄ alkyl optionally substituted with F.

In some particular embodiments, R² in Formula I (e.g., Formula I-1 or I-2) can be cyclopropyl optionally substituted with 1-4 substituents independently selected from CN, halogen, OH, and optionally substituted C₁₋₆ alkyl. In some embodiments, R² in Formula I (e.g., Formula I-1 or I-2) can be cyclopropyl optionally substituted with 1-4 (e.g., 1, 2, or 3) substituents independently selected from CN, F, Cl, OH, and C₁₋₄ alkyl optionally substituted with F.

In some preferred embodiments, R² in Formula I (e.g., Formula I-1 or I-2) can be cyclopropyl.

The phenyl group drawn in Formula I (e.g., Formula I-1 or I-2) can be optionally further substituted with 1-3 independently selected R³ groups, i.e., j is 0, 1, 2, or 3. In some embodiments, j is 0. In some embodiments, j is 1. In some embodiments, j is 2. In some embodiments, one R³ group is substituted at a position para to the R⁴ group. When present, R³ at each occurrence can be independently halogen, optionally substituted C₁₋₄ alkyl, or optionally substituted C₁₋₄ heteroalkyl. In some embodiments, R³ at each occurrence can be independently F, Cl, or C₁₋₄ alkyl optionally substituted with F. In some embodiments, j is 1, and R³ can be a halogen, such as F. In some embodiments, j is 1, and R³ can be an optionally substituted C₁₋₄ alkyl, such as methyl or ethyl, optionally substituted with F.

Various groups are suitable as R⁴ in Formula I (e.g., Formula I-1 or I-2). In some embodiments, R⁴ is a C₁₋₆ alkyl optionally substituted with 1-3 R^{A}, wherein R^{A} at each occurrence is independently halogen, OH, C₁₋₆ alkyl optionally substituted with 1-3 R^{A1}, wherein R^{A1} at each occurrence is independently halogen, OH, or CN. For example, in some embodiments, R⁴ is a C₁₋₄ alkyl optionally substituted with 1-3 R^{A}, wherein R^{A} at each occurrence is independently F, OH, or C₁₋₄ alkyl optionally substituted with 1-3 F.

In some embodiments, R⁴ in Formula I (e.g., Formula I-1 or I-2) is S(O)ₚR^{B}, wherein p is 0, 1 or 2, and R^{B} is C₁₋₆ alkyl optionally substituted with 1-3 R^{A1}, wherein R^{A1} at each occurrence is independently halogen, OH, or CN. For example, in some embodiments, R⁴ in Formula I (e.g., Formula I-1 or I-2) is S(O)₂R^{B}, and R^{B} is C₁₋₄ alkyl optionally substituted with 1-3 R_{A1}, wherein R^{A1} at each occurrence is independently F, OH, or CN.

In some embodiments, R⁴ in Formula I (e.g., Formula I-1 or I-2) is OR^{C}, wherein R^{C} is hydrogen or C₁₋₆ alkyl optionally substituted with 1-3 R^{A2}, wherein R^{A2} at each occurrence is independently F or OH. For example, in some embodiments, R⁴ in Formula I (e.g., Formula I-1 or I-2) is OH. In some embodiments, R⁴ in Formula I (e.g., Formula I-1 or I-2) is OR^{C}, wherein R^{C} is a C₁₋₄ alkyl optionally substituted with 1-3 R^{A2}, wherein R^{A2} at each occurrence is independently F or OH. In some embodiments, R⁴ in Formula I (e.g., Formula I-1 or I-2) is OR^{C}, wherein R^{C} is a C₁₋₄ alkyl such as methyl. In some embodiments, R⁴ in Formula I (e.g., Formula I-1 or I-2) is OR^{C}, wherein R^{C} is a C₁₋₄ alkyl optionally substituted with 1-3 F.

In some preferred embodiments, R⁴ in Formula I (e.g., Formula I-1 or I-2) is OMe. For example, in some embodiments, the compound of Formula I can be characterized as having a Formula I-1-A, I-1-A-1, I-1-A-2, I-1-A-3, I-1-A-4, I-2-A, I-2-A-1, I-2-A-2, I-2-A-3, or I-2-A-4:

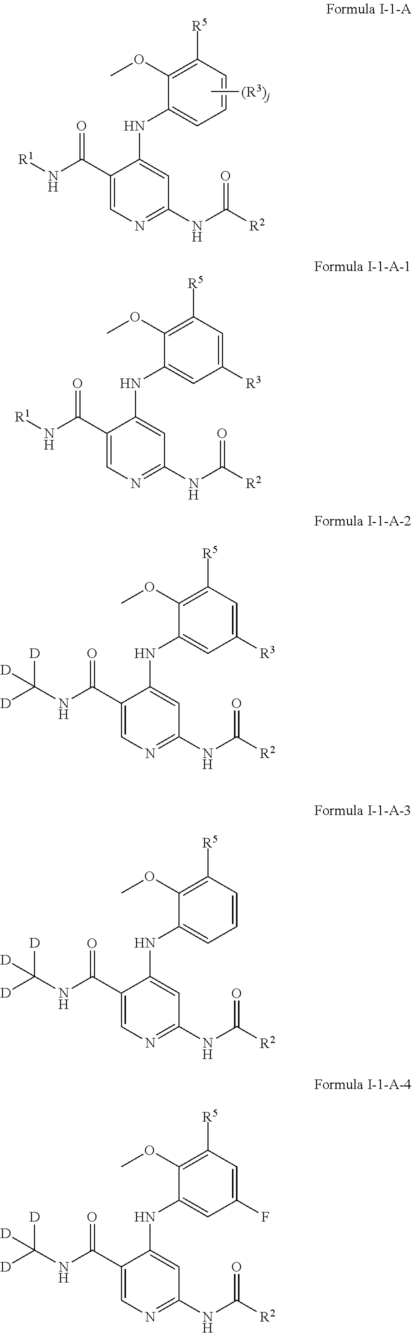

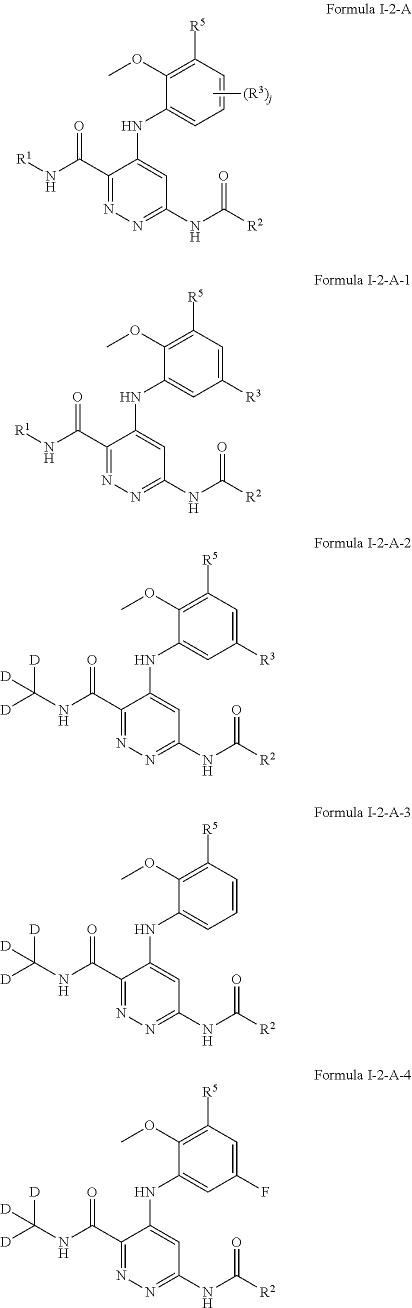

wherein j, R¹, R², R³, and R⁵ include any of those described herein in any combination.

In some embodiments, R⁵ in Formula I (e.g., Formula I-1, I-2, or any of the applicable subformulae described herein) can be an optionally substituted heteroaryl, such as a substituted 5 or 6-membered heteroaryl having 1-4 ring heteroatoms, for example, substituted pyridyl, substituted pyrimidinyl, substituted triazolyl, etc. When substituted, the heteroaryl is typically substituted with 1-2 substituents independently selected from halogen, OH, CN, optionally substituted C₁₋₆ alkyl, optionally substituted C₁₋₆ heteroalkyl, optionally substituted cycloalkyl (e.g., C₃₋₆ cycloalkyl), and optionally substituted heterocyclyl (e.g., 4-8 membered heterocyclyl). For example, typically, the heteroaryl can be substituted with 1 or 2 substituents independently selected from F, Cl, OH, CN, C₁₋₄ alkyl optionally substituted with 1-3 S^{A}, C₁₋₄ alkoxyl optionally substituted with 1-3 S^{A}, C₃₋₆ cycloalkyl optionally substituted with 1-3 S^{B}, and 4-8 membered heterocyclyl optionally substituted with 1-3 S^{B}, wherein S^{A} at each occurrence is independently F, OH, or C₁₋₄ alkoxy, S^{B} at each occurrence is independently oxo, F, C₁₋₄ alkyl optionally substituted with F, OH, or C₁₋₄ heteroalkyl optionally substituted with F. Examples of optionally substituted heteroaryls as R⁵ include those exemplified herein.

In some embodiments, R⁵ in Formula I (e.g., Formula I-1, I-2, or any of the applicable subformulae described herein) can be an optionally substituted heterocyclyl. In some embodiments, R⁵ in Formula I (e.g., Formula I-1, I-2, or any of the applicable subformulae described herein) can be an optionally substituted monocyclic (e.g., monocyclic 4-8 membered) heterocyclyl group having 1-4 ring heteroatoms independently selected from N, O, and S. In some embodiments, R⁵ in Formula I (e.g., Formula I-1, I-2, or any of the applicable subformulae described herein) can be an optionally substituted heterocyclyl group having 1-4 ring heteroatoms independently selected from N, O, and S, which includes an spiro, bridged, or fused ring system, such as a 6-10 membered spiro, bridged, or fused bicyclic heterocyclyl. When substituted, the heterocyclyl is typically substituted with 1-2 substituents independently selected from oxo, halogen, OH, CN, optionally substituted C₁₋₆ alkyl, optionally substituted C₁₋₆ heteroalkyl, optionally substituted cycloalkyl (e.g., C₃₋₆ cycloalkyl), and optionally substituted heterocyclyl (e.g., 4-8 membered heterocyclyl). For example, typically, the heterocyclyl can be substituted with 1 or 2 substituents independently selected from F, Cl, OH, CN, C₁₋₄ alkyl optionally substituted with 1-3 S^{A}, C₁₋₄ alkoxyl optionally substituted with 1-3 S^{A}, C₃₋₆ cycloalkyl optionally substituted with 1-3 S^{B}, and 4-8 membered heterocyclyl optionally substituted with 1-3 S^{B}, wherein S^{A} at each occurrence is independently F, OH, or C₁₋₄ alkoxy, S^{B} at each occurrence is independently oxo, F, C₁₋₄ alkyl optionally substituted with F, OH, or C₁₋₄ heteroalkyl optionally substituted with F. Examples of optionally substituted heterocyclyl as R⁵ include those exemplified herein.

In some embodiments, R⁵ in Formula I (e.g., Formula I-1, I-2, or any of the applicable subformulae described herein) is a substituted pyrimidine, e.g., with one substituent para to the phenyl group in Formula I and optionally one or more additional substituents. For example, in some embodiments, R⁵ in Formula I (e.g., Formula I-1, I-2, or any of the applicable subformulae described herein) can be

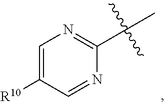

wherein R¹⁰ is an optionally substituted cycloalkyl (e.g., C₃₋₆ cycloalkyl), or optionally substituted heterocyclyl (e.g., 4-8 membered heterocyclyl). For example, in some embodiments, R¹⁰ is an optionally substituted cycloalkyl, such as an optionally substituted C₃₋₆ cycloalkyl. In some specific embodiments, R¹⁰ can be

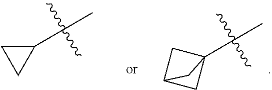

In some embodiments, R⁵ in Formula I (e.g., Formula I-1, I-2, or any of the applicable subformulae described herein) is a substituted pyridine, e.g., with one substituent para to the phenyl group in Formula I and optionally one or more additional substituents. For example, in some embodiments, R⁵ in Formula I (e.g., Formula I-1, I-2, or any of the applicable subformulae described herein) can be

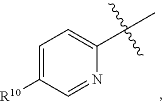

wherein R¹⁰ is an optionally substituted cycloalkyl (e.g., C₃₋₆ cycloalkyl), or optionally substituted heterocyclyl (e.g., 4-8 membered heterocyclyl). For example, in some embodiments, R¹⁰ is an optionally substituted cycloalkyl, such as an optionally substituted C₃₋₆ cycloalkyl. In some specific embodiments, R¹⁰ can be

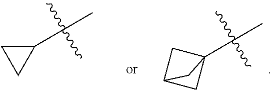

In some embodiments, R⁵ in Formula I (e.g., Formula I-1, I-2, or any of the applicable subformulae described herein) is a substituted pyrimidinone, e.g., with one substituent para to the phenyl group in Formula I and optionally one or more additional substituents. For example, in some embodiments, R⁵ in Formula I (e.g., Formula I-1, I-2, or any of the applicable subformulae described herein) can be

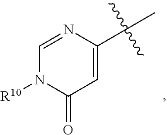

wherein R¹⁰ is an optionally substituted cycloalkyl (e.g., C₃₋₆ cycloalkyl), or optionally substituted heterocyclyl (e.g., 4-8 membered heterocyclyl). For example, in some embodiments, R¹⁰ is an optionally substituted cycloalkyl, such as an optionally substituted C₃₋₆ cycloalkyl. In some specific embodiments, R¹⁰ can be

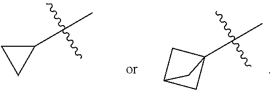

In some embodiments, R⁵ in Formula I (e.g., Formula I-1, I-2, or any of the applicable subformulae described herein) is an optionally substituted triazole, such as 1,2,3-triazole or 1,2,4-triazole. Typically, the triazole connects to the phenyl group in Formula I through a ring nitrogen atom. For example, in some embodiments, R⁵ in Formula I (e.g., Formula I-1, I-2, or any of the applicable subformulae described herein) can be a substituted 1,2,4-triazole such as

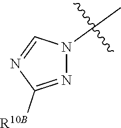

wherein R^{10B} is halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) can be a substituted 1,2,3-triazole such as

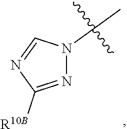

wherein R^{10B} is halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) can be a substituted 1,2,3-triazole such as

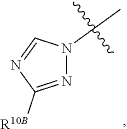

wherein R^{10B} is halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) is an optionally substituted imidazolone. For example, in some embodiments, R⁵ in Formula I (e.g., Formula I-1, I-2, or any of the applicable subformulae described herein) can be a 3,5-dihydro-4H-imidazol-4-one, wherein the 5-position is a quaternary center, such as

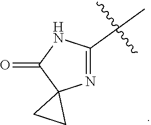 [SMILES: *C1=NC2(CC2)C(=O)N1]

In some embodiments, R⁵ in Formula I (e.g., Formula I-1, I-2, or any of the applicable subformulae described herein) is an optionally substituted pyrazolone. For example, in some embodiments, R⁵ in Formula I (e.g., Formula I-1, I-2, or any of the applicable subformulae described herein) can be a substituted 2,4-dihydro-3H-pyrazol-3-one such as

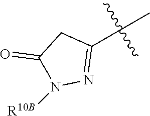

wherein R^{10B} is an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) is an optionally substituted oxadiazolone. For example, in some embodiments, R⁵ in Formula I (e.g., Formula I-1, I-2, or any of the applicable subformulae described herein) can be a substituted 1,3,4-oxadiazol-2(3H)-one such as

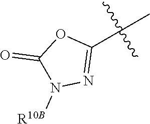

wherein R^{10B} is an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) is an optionally substituted triazolone. For example, in some embodiments, R⁵ in Formula I (e.g., Formula I-1, I-2, or any of the applicable subformulae described herein) can be a substituted 1,2-dihydro-3H-1,2,4-triazol-3-one such as

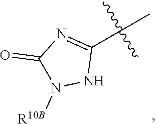

wherein R^{10B} is an optionally substituted C₁₋₆ alkyl, an optionally substituted cycloalkyl (e.g., C₃₋₆ cycloalkyl), or optionally substituted heterocyclyl (e.g., 4-8 membered heterocyclyl). In some embodiments, R^{10B} is an optionally substituted C₁₋₄ alkyl or an optionally substituted C₃₋₆ cycloalkyl. In some specific embodiments, R^{10B} can be methyl, CF₃, or cyclopropyl.

In some embodiments, R⁵ in Formula I (e.g., Formula I-1, I-2, or any of the applicable subformulae described herein) can also be characterized as having a formula according to -L¹-L²-Q-G as defined herein. For example, in some embodiments, the compound of Formula I can be characterized as having a Formula I-1-B or I-2-B:

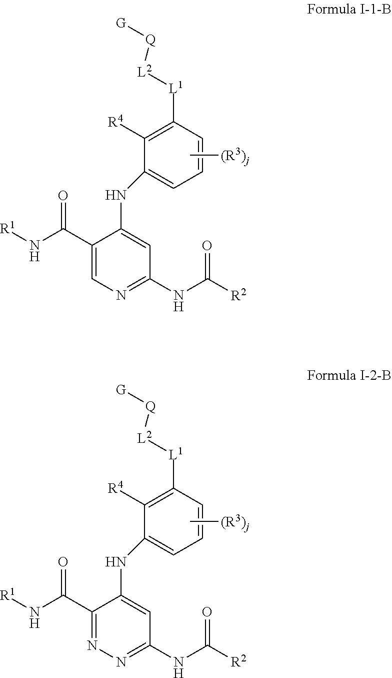

wherein L¹, L², Q, G, j, R¹, R², R³, and R⁴ include any of those described herein in any combination.

In some embodiments, L¹ is O.

In some embodiments, L¹ is C═(O)NH, such as

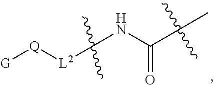

(G, Q, and L2 are shown to show direction of connection with the remainder of the molecule).

In some embodiments, L¹ is null.

In some embodiments, L² is C₁₋₄ alkylene, such as methylene.

In some embodiments, L² is null.

Q is typically an optionally substituted heterocycle. For example, in some embodiments, Q is a 4-7 membered monocyclic heterocyclic ring having 1 or 2 ring heteroatoms independently selected from N, O, and S, which is optionally substituted. In some embodiments, when substituted, the 4-7 membered monocyclic heterocyclic ring can be substituted with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. In some embodiments, Q can be a 4-7 membered monocyclic heterocyclic ring selected from:

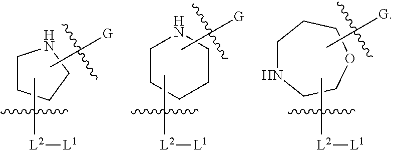

In some preferred embodiments, Q can be a 4-7 membered monocyclic heterocyclic ring selected from:

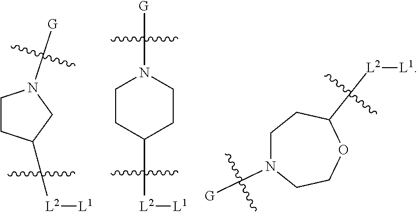

In some embodiments, Q can also be a 6-12 membered bicyclic heterocyclic ring having 1-4 ring heteroatoms independently selected from N, O, and S, which is optionally substituted. In some embodiments, when substituted, the 6-12 membered bicyclic heterocyclic ring can be substituted with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. The 6-12 membered bicyclic heterocyclic ring can be a fused, spiro, or bridged bicyclic ring, wherein one of the rings is optionally aromatic or heteroaromatic. For example, in some embodiments, Q can be a 6-10 membered bicyclic heterocyclic ring selected from:

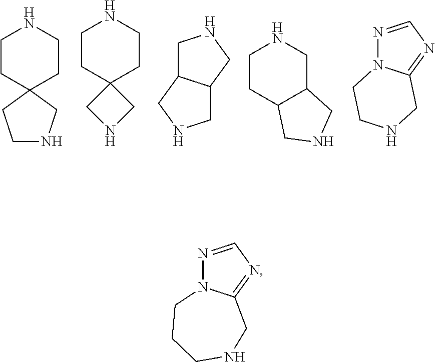

wherein each of which is optionally substituted, for example, with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. In some embodiments, Q can be a 6-10 membered bicyclic heterocyclic ring selected from:

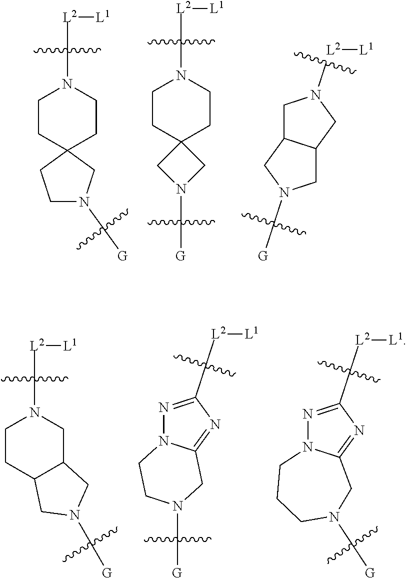

In some embodiments, Q can also be an optionally substituted heteroaryl. For example, in some embodiments, Q is a 5 or 6 membered heteroaryl having 1-4 ring heteroatoms independently selected from N, O, and S, which is optionally substituted, for example, with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. In some embodiments, Q is pyridine or pyrimidine, which is optionally substituted, for example, with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F. In some embodiments, Q is

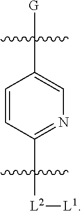

In some embodiments, G is CN.

In some embodiments, G is

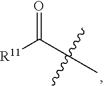

wherein R¹¹ is hydrogen, an optionally substituted C₁₋₆ alkyl or an optionally substituted C₃₋₆ cycloalkyl. In some embodiments, R¹¹ is hydrogen or C₁₋₄ alkyl, such as methyl, optionally substituted with halogen, such as F or Cl.

In some embodiments, G is

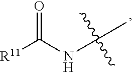

wherein R¹¹ is hydrogen, an optionally substituted C₁₋₆ alkyl or an optionally substituted C₃₋₆ cycloalkyl. In some embodiments, R¹¹ is hydrogen or C₁₋₄ alkyl, such as methyl, optionally substituted with halogen, such as F or Cl.

In some embodiments, G is a Michael acceptor. Typically, when G is a Michael acceptor, it contains an alpha-beta unsaturated carbonyl group. For example, in some embodiments, G can be

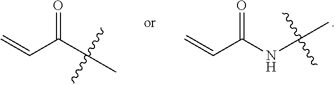

The combination of L¹, L², Q and G is not particularly limited. For example, in some embodiments, the compound of Formula I can be characterized as having a Formula I-1-B-1, I-1-B-2, I-1-B-3, I-2-B-1, I-2-B-2, or I-2-B-3:

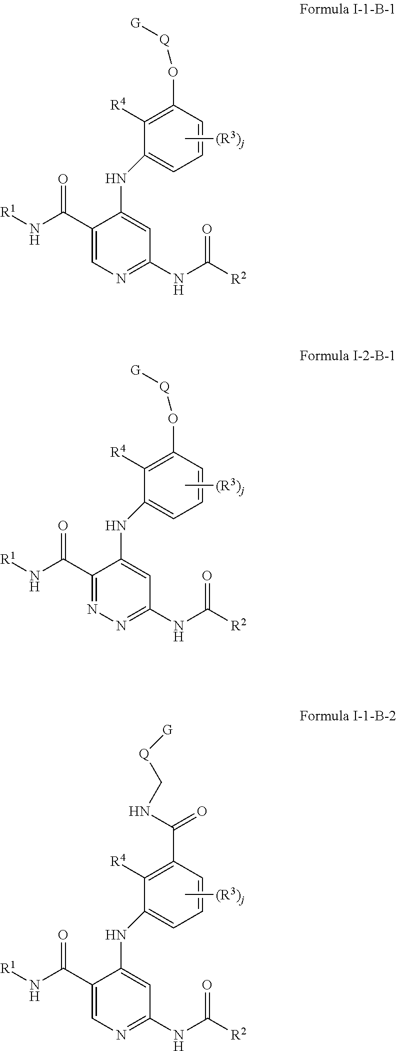

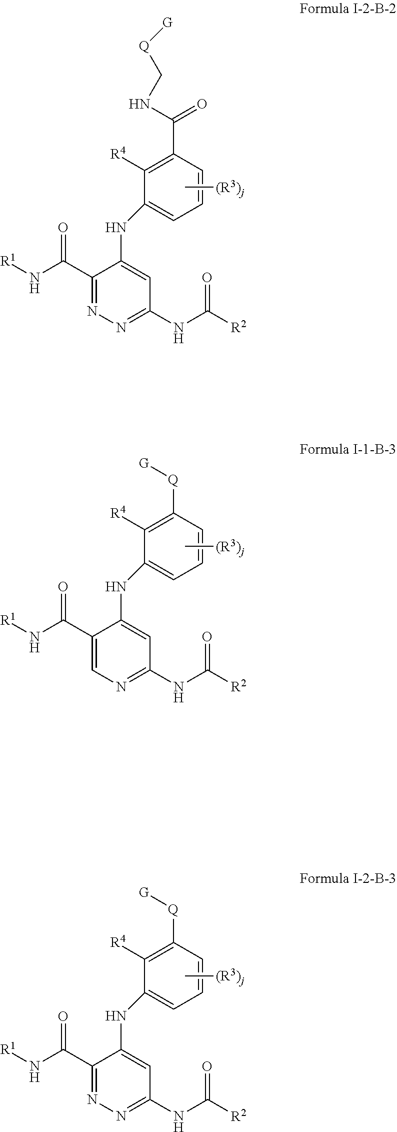

wherein Q, G, j, R¹, R², R³, and R⁴ include any of those described herein in any combination. Exemplary combinations of L¹, L², Q and G are also shown herein such as in the compounds of Table 1.

In some embodiments, R⁵ in Formula I (e.g., Formula I-1, I-2, or any of the applicable subformulae described herein) can be

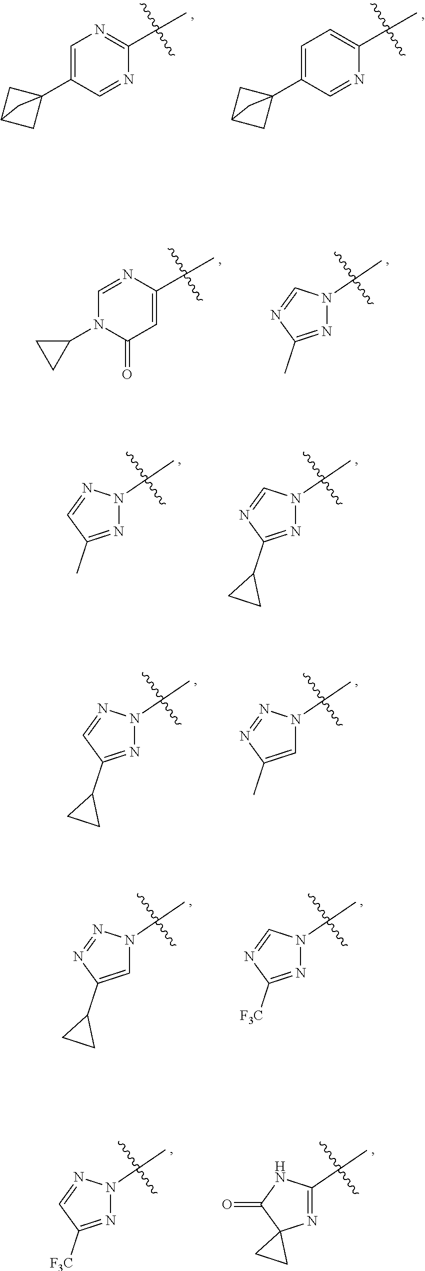

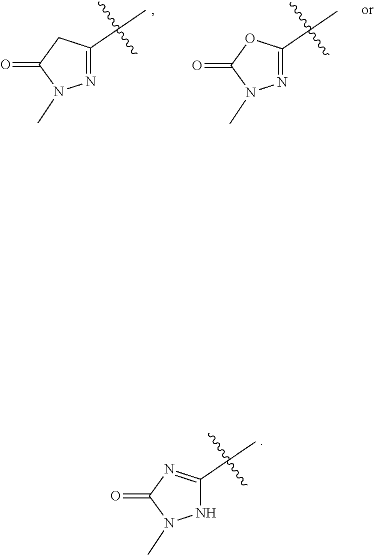

In some embodiments, R⁵ in Formula I (e.g., Formula I-1, I-2, or any of the applicable subformulae described herein) can be selected from:

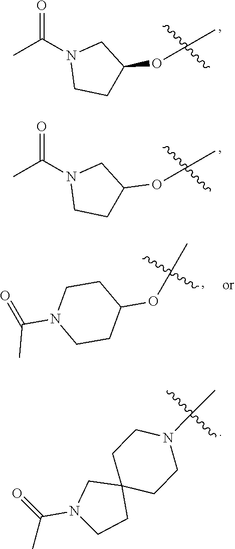

In some embodiments, R⁵ in Formula I (e.g., Formula I-1, I-2, or any of the applicable subformulae described herein) can be selected from:

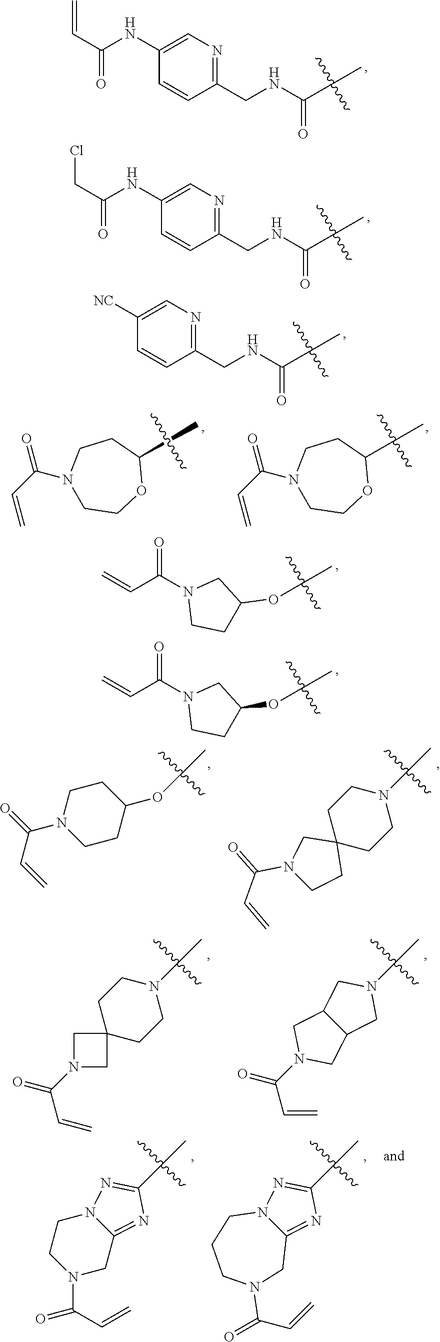

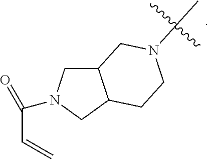 [SMILES: *N1CCC2CN(C(=O)C=C)CC2C1]

In some embodiments, the present disclosure also provide a compound selected from the compounds shown in Table 1, or a pharmaceutically acceptable salt thereof:

TABLE 1
List of Compounds

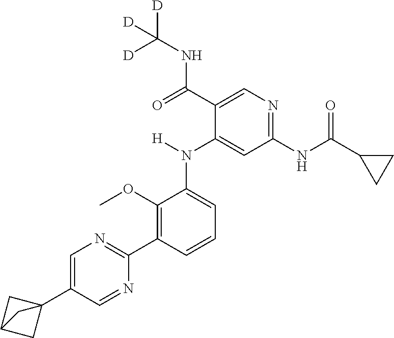 [SMILES: [2H]C([2H])([2H])NC(=O)c1cnc(NC(=O)C2CC2)cc1Nc1cccc(-c2ncc(C34CC(C3)C4)cn2)c1OC]

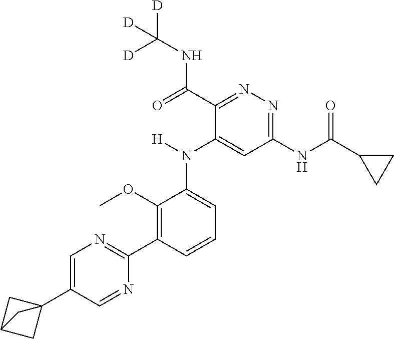 [SMILES: [2H]C([2H])([2H])NC(=O)c1nnc(NC(=O)C2CC2)cc1Nc1cccc(-c2ncc(C34CC(C3)C4)cn2)c1OC]

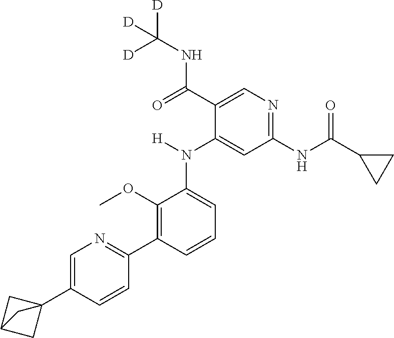 [SMILES: [2H]C([2H])([2H])NC(=O)c1cnc(NC(=O)C2CC2)cc1Nc1cccc(-c2ccc(C34CC(C3)C4)cn2)c1OC]

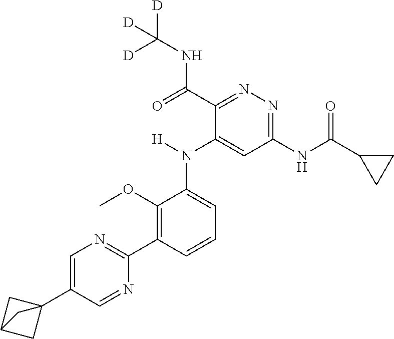 [SMILES: [2H]C([2H])([2H])NC(=O)c1nnc(NC(=O)C2CC2)cc1Nc1cccc(-c2ncc(C34CC(C3)C4)cn2)c1OC]

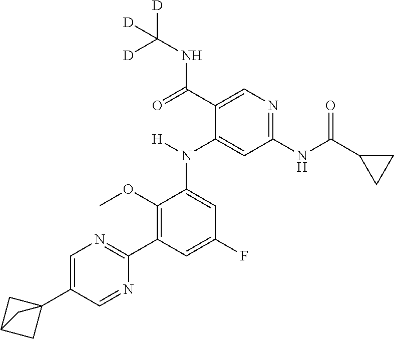 [SMILES: [2H]C([2H])([2H])NC(=O)c1cnc(NC(=O)C2CC2)cc1Nc1cc(F)cc(-c2ncc(C34CC(C3)C4)cn2)c1OC]

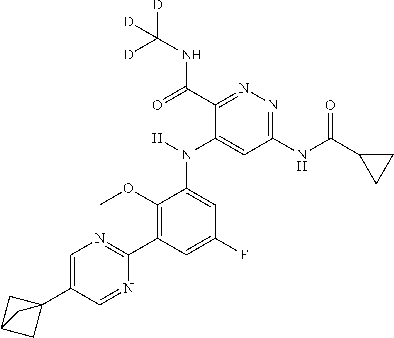 [SMILES: [2H]C([2H])([2H])NC(=O)c1nnc(NC(=O)C2CC2)cc1Nc1cc(F)cc(-c2ncc(C34CC(C3)C4)cn2)c1OC]

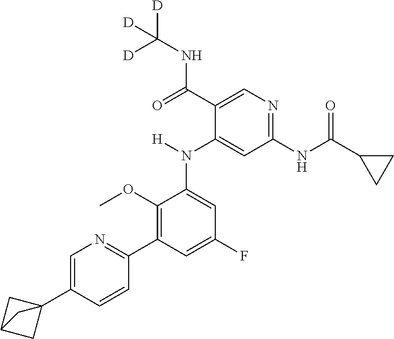 [SMILES: [2H]C([2H])([2H])NC(=O)c1cnc(NC(=O)C2CC2)cc1Nc1cc(F)cc(-c2ccc(C34CC(C3)C4)cn2)c1OC]

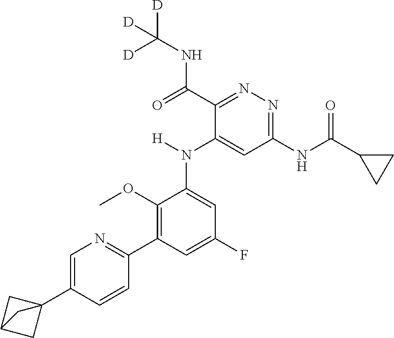 [SMILES: [2H]C([2H])([2H])NC(=O)c1nnc(NC(=O)C2CC2)cc1Nc1cc(F)cc(-c2ccc(C34CC(C3)C4)cn2)c1OC]

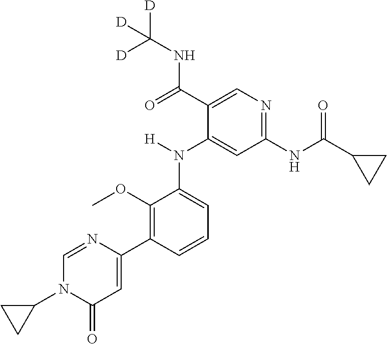 [SMILES: [2H]C([2H])([2H])NC(=O)c1cnc(NC(=O)C2CC2)cc1Nc1cccc(-c2cc(=O)n(C3CC3)cn2)c1OC]

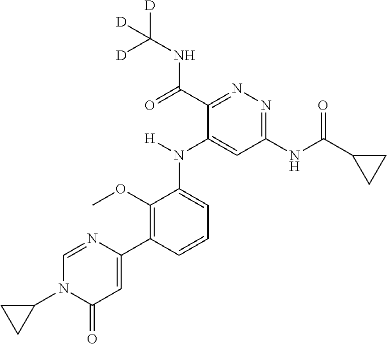 [SMILES: [2H]C([2H])([2H])NC(=O)c1nnc(NC(=O)C2CC2)cc1Nc1cccc(-c2cc(=O)n(C3CC3)cn2)c1OC]

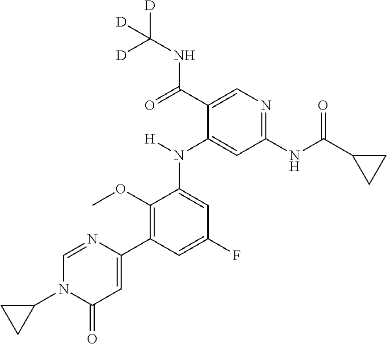 [SMILES: [2H]C([2H])([2H])NC(=O)c1cnc(NC(=O)C2CC2)cc1Nc1cc(F)cc(-c2cc(=O)n(C3CC3)cn2)c1OC]

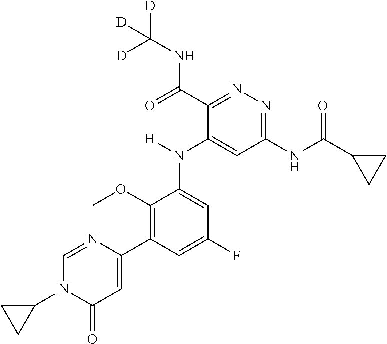 [SMILES: [2H]C([2H])([2H])NC(=O)c1nnc(NC(=O)C2CC2)cc1Nc1cc(F)cc(-c2cc(=O)n(C3CC3)cn2)c1OC]

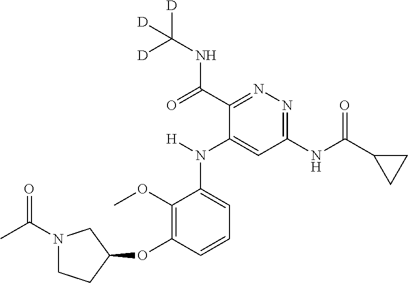 [SMILES: [2H]C([2H])([2H])NC(=O)c1nnc(NC(=O)C2CC2)cc1Nc1cccc(O[C@H]2CCN(C(C)=O)C2)c1OC]

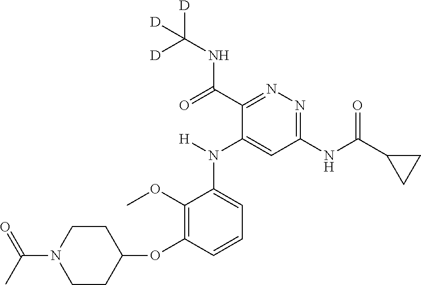 [SMILES: [2H]C([2H])([2H])NC(=O)c1nnc(NC(=O)C2CC2)cc1Nc1cccc(OC2CCN(C(C)=O)CC2)c1OC]

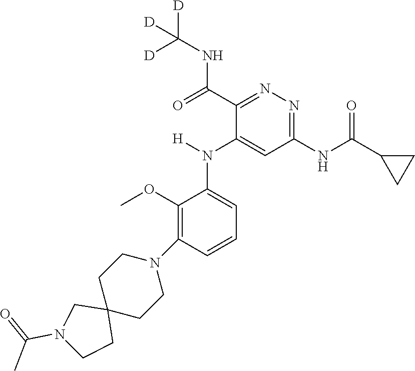 [SMILES: [2H]C([2H])([2H])NC(=O)c1nnc(NC(=O)C2CC2)cc1Nc1cccc(N2CCC3(CCN(C(C)=O)C3)CC2)c1OC]

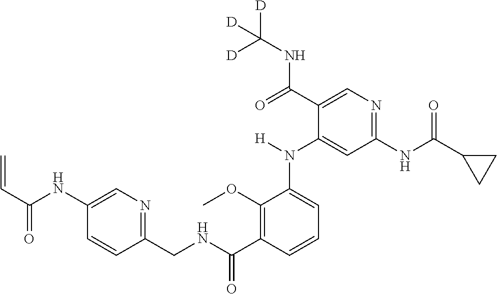 [SMILES: [2H]C([2H])([2H])NC(=O)c1cnc(NC(=O)C2CC2)cc1Nc1cccc(C(=O)NCc2ccc(NC(=O)C=C)cn2)c1OC]

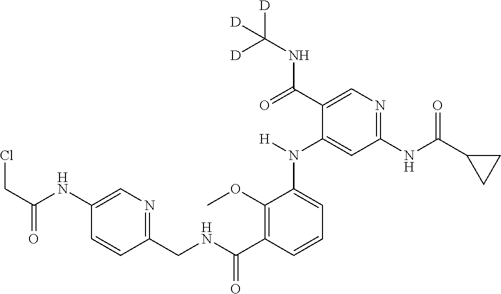 [SMILES: [2H]C([2H])([2H])NC(=O)c1cnc(NC(=O)C2CC2)cc1Nc1cccc(C(=O)NCc2ccc(NC(=O)CCl)cn2)c1OC]

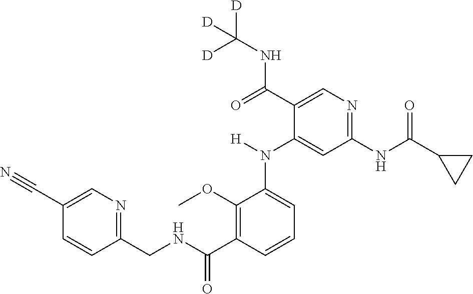 [SMILES: [2H]C([2H])([2H])NC(=O)c1cnc(NC(=O)C2CC2)cc1Nc1cccc(C(=O)NCc2ccc(C#N)cn2)c1OC]

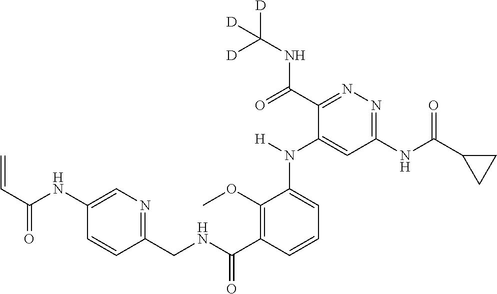 [SMILES: [2H]C([2H])([2H])NC(=O)c1nnc(NC(=O)C2CC2)cc1Nc1cccc(C(=O)NCc2ccc(NC(=O)C=C)cn2)c1OC]

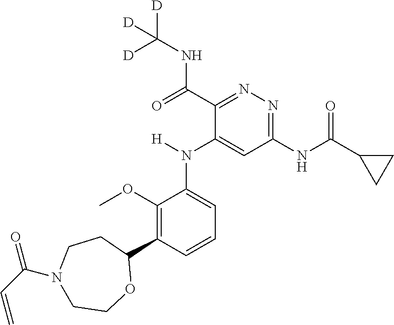

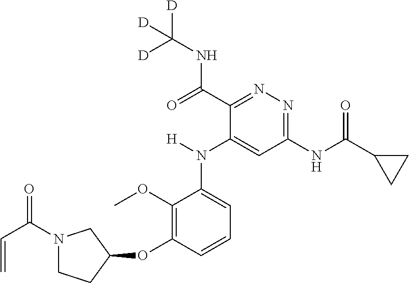 [SMILES: [2H]C([2H])([2H])NC(=O)c1nnc(NC(=O)C2CC2)cc1Nc1cccc(O[C@H]2CCN(C(=O)C=C)C2)c1OC]

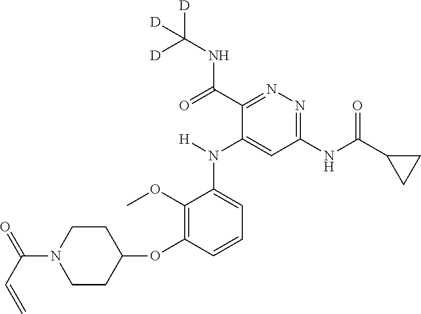 [SMILES: [2H]C([2H])([2H])NC(=O)c1nnc(NC(=O)C2CC2)cc1Nc1cccc(OC2CCN(C(=O)C=C)CC2)c1OC]

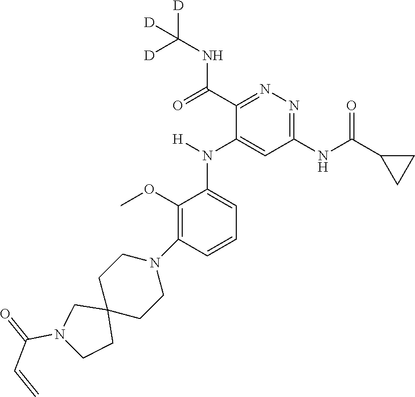 [SMILES: [2H]C([2H])([2H])NC(=O)c1nnc(NC(=O)C2CC2)cc1Nc1cccc(N2CCC3(CCN(C(=O)C=C)C3)CC2)c1OC]

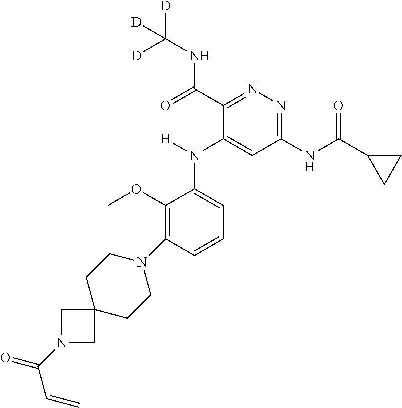 [SMILES: [2H]C([2H])([2H])NC(=O)c1nnc(NC(=O)C2CC2)cc1Nc1cccc(N2CCC3(CC2)CN(C(=O)C=C)C3)c1OC]

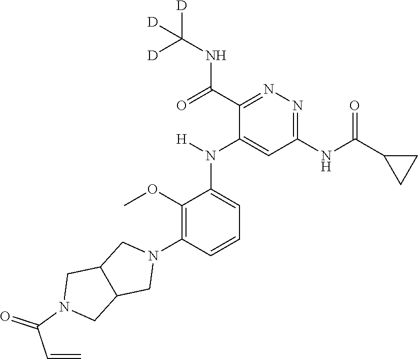 [SMILES: [2H]C([2H])([2H])NC(=O)c1nnc(NC(=O)C2CC2)cc1Nc1cccc(N2CC3CN(C(=O)C=C)CC3C2)c1OC]

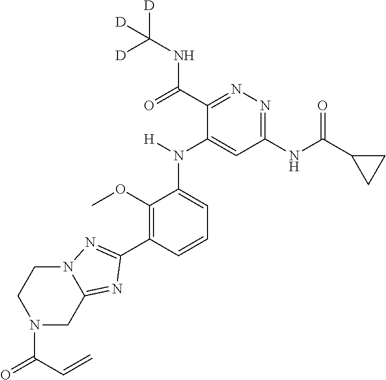 [SMILES: [2H]C([2H])([2H])NC(=O)c1nnc(NC(=O)C2CC2)cc1Nc1cccc(-c2nc3n(n2)CCN(C(=O)C=C)C3)c1OC]

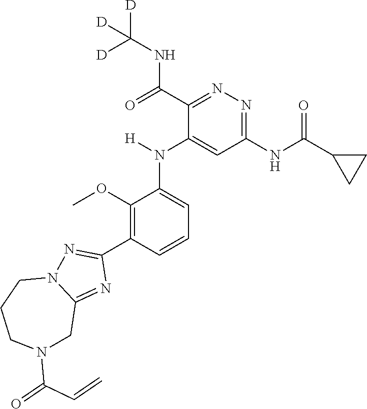 [SMILES: [2H]C([2H])([2H])NC(=O)c1nnc(NC(=O)C2CC2)cc1Nc1cccc(-c2nc3n(n2)CCCN(C(=O)C=C)C3)c1OC]

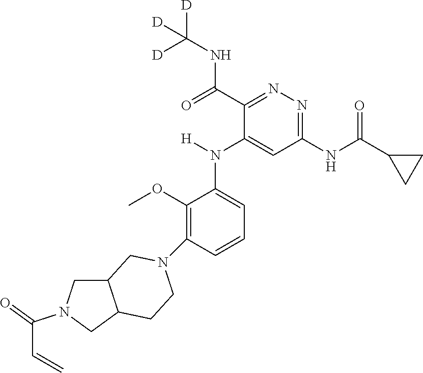 [SMILES: [2H]C([2H])([2H])NC(=O)c1nnc(NC(=O)C2CC2)cc1Nc1cccc(N2CCC3CN(C(=O)C=C)CC3C2)c1OC]

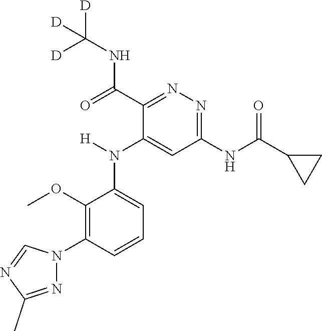 [SMILES: [2H]C([2H])([2H])NC(=O)c1nnc(NC(=O)C2CC2)cc1Nc1cccc(-n2cnc(C)n2)c1OC]

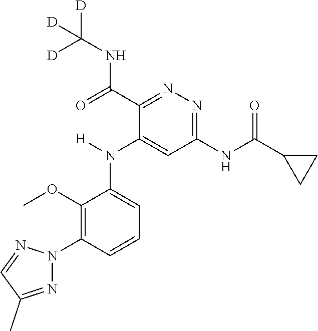 [SMILES: [2H]C([2H])([2H])NC(=O)c1nnc(NC(=O)C2CC2)cc1Nc1cccc(-n2ncc(C)n2)c1OC]

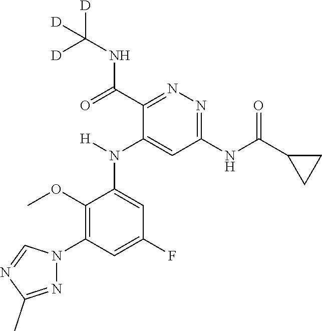 [SMILES: [2H]C([2H])([2H])NC(=O)c1nnc(NC(=O)C2CC2)cc1Nc1cc(F)cc(-n2cnc(C)n2)c1OC]

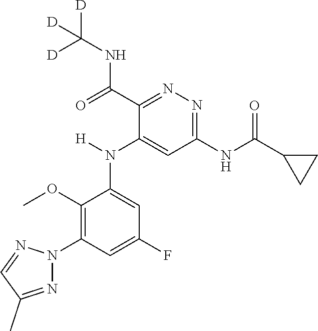 [SMILES: [2H]C([2H])([2H])NC(=O)c1nnc(NC(=O)C2CC2)cc1Nc1cc(F)cc(-n2ncc(C)n2)c1OC]

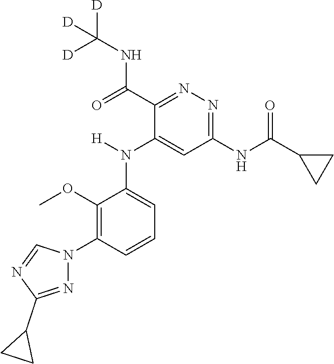 [SMILES: [2H]C([2H])([2H])NC(=O)c1nnc(NC(=O)C2CC2)cc1Nc1cccc(-n2cnc(C3CC3)n2)c1OC]

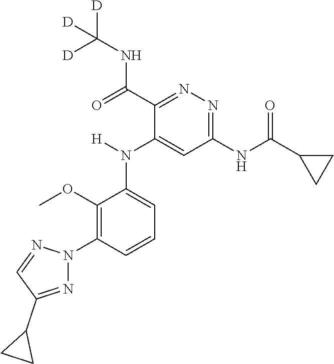 [SMILES: [2H]C([2H])([2H])NC(=O)c1nnc(NC(=O)C2CC2)cc1Nc1cccc(-n2ncc(C3CC3)n2)c1OC]

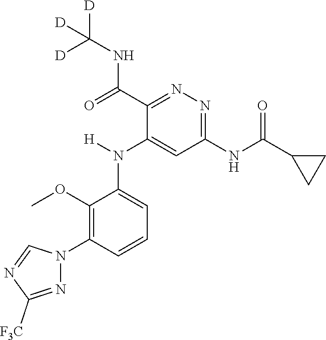 [SMILES: [2H]C([2H])([2H])NC(=O)c1nnc(NC(=O)C2CC2)cc1Nc1cccc(-n2cnc(C(F)(F)F)n2)c1OC]

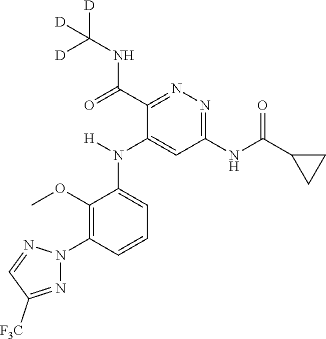 [SMILES: [2H]C([2H])([2H])NC(=O)c1nnc(NC(=O)C2CC2)cc1Nc1cccc(-n2ncc(C(F)(F)F)n2)c1OC]

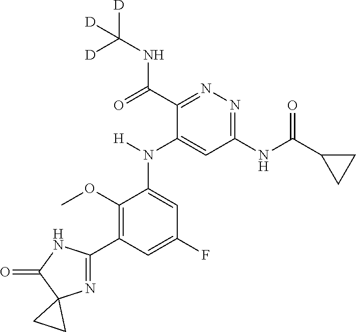 [SMILES: [2H]C([2H])([2H])NC(=O)c1nnc(NC(=O)C2CC2)cc1Nc1cc(F)cc(C2=NC3(CC3)C(=O)N2)c1OC]

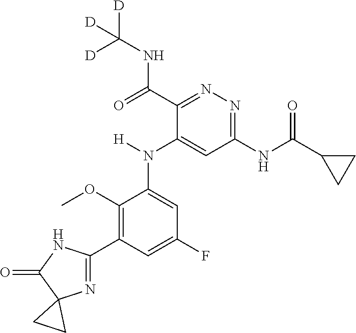 [SMILES: [2H]C([2H])([2H])NC(=O)c1nnc(NC(=O)C2CC2)cc1Nc1cc(F)cc(C2=NC3(CC3)C(=O)N2)c1OC]

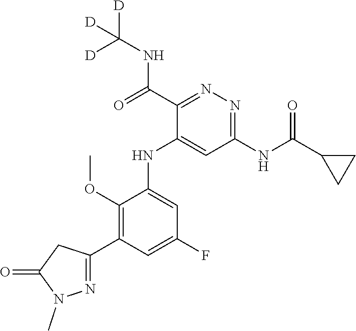 [SMILES: [2H]C([2H])([2H])NC(=O)c1nnc(NC(=O)C2CC2)cc1Nc1cc(F)cc(C2=NN(C)C(=O)C2)c1OC]

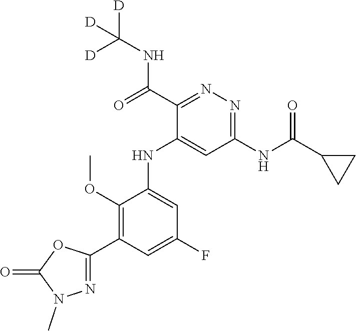 [SMILES: [2H]C([2H])([2H])NC(=O)c1nnc(NC(=O)C2CC2)cc1Nc1cc(F)cc(-c2nn(C)c(=O)o2)c1OC]

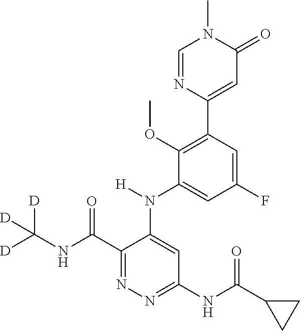 [SMILES: [2H]C([2H])([2H])NC(=O)c1nnc(NC(=O)C2CC2)cc1Nc1cc(F)cc(-c2cc(=O)n(C)cn2)c1OC]

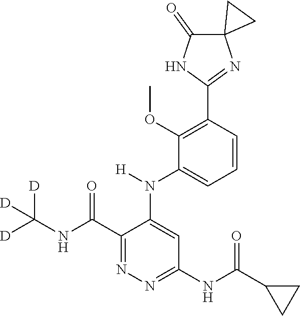 [SMILES: [2H]C([2H])([2H])NC(=O)c1nnc(NC(=O)C2CC2)cc1Nc1cccc(C2=NC3(CC3)C(=O)N2)c1OC]

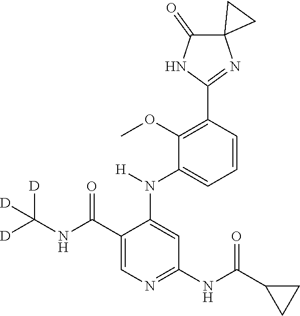 [SMILES: [2H]C([2H])([2H])NC(=O)c1cnc(NC(=O)C2CC2)cc1Nc1cccc(C2=NC3(CC3)C(=O)N2)c1OC]

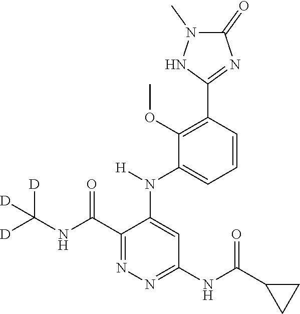 [SMILES: [2H]C([2H])([2H])NC(=O)c1nnc(NC(=O)C2CC2)cc1Nc1cccc(-c2nc(=O)n(C)[nH]2)c1OC]

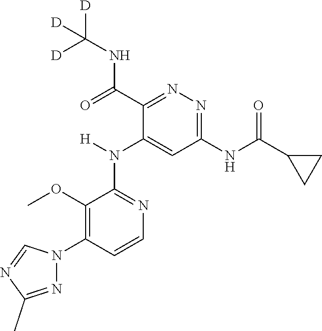 [SMILES: [2H]C([2H])([2H])NC(=O)c1nnc(NC(=O)C2CC2)cc1Nc1nccc(-n2cnc(C)n2)c1OC]

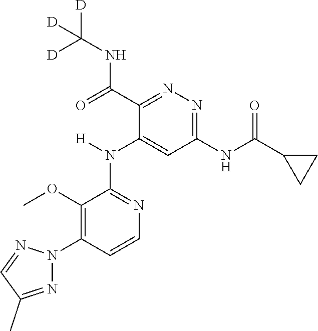 [SMILES: [2H]C([2H])([2H])NC(=O)c1nnc(NC(=O)C2CC2)cc1Nc1nccc(-n2ncc(C)n2)c1OC]

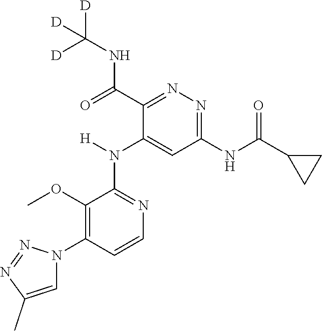 [SMILES: [2H]C([2H])([2H])NC(=O)c1nnc(NC(=O)C2CC2)cc1Nc1nccc(-n2cc(C)nn2)c1OC]

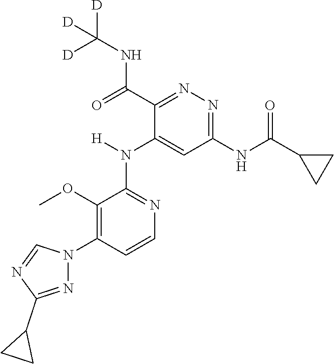 [SMILES: [2H]C([2H])([2H])NC(=O)c1nnc(NC(=O)C2CC2)cc1Nc1nccc(-n2cnc(C3CC3)n2)c1OC]

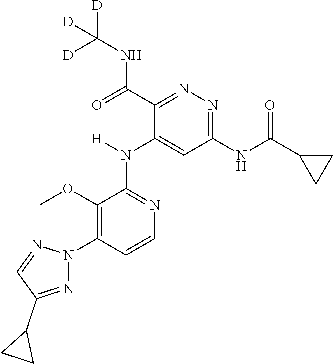 [SMILES: [2H]C([2H])([2H])NC(=O)c1nnc(NC(=O)C2CC2)cc1Nc1nccc(-n2ncc(C3CC3)n2)c1OC]

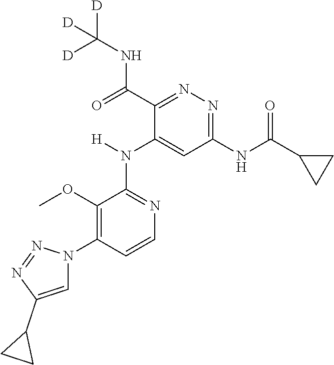 [SMILES: [2H]C([2H])([2H])NC(=O)c1nnc(NC(=O)C2CC2)cc1Nc1nccc(-n2cc(C3CC3)nn2)c1OC]

In some embodiments, the present disclosure also provide a compound selected from the compounds of Examples 1-15, or a pharmaceutically acceptable salt thereof:

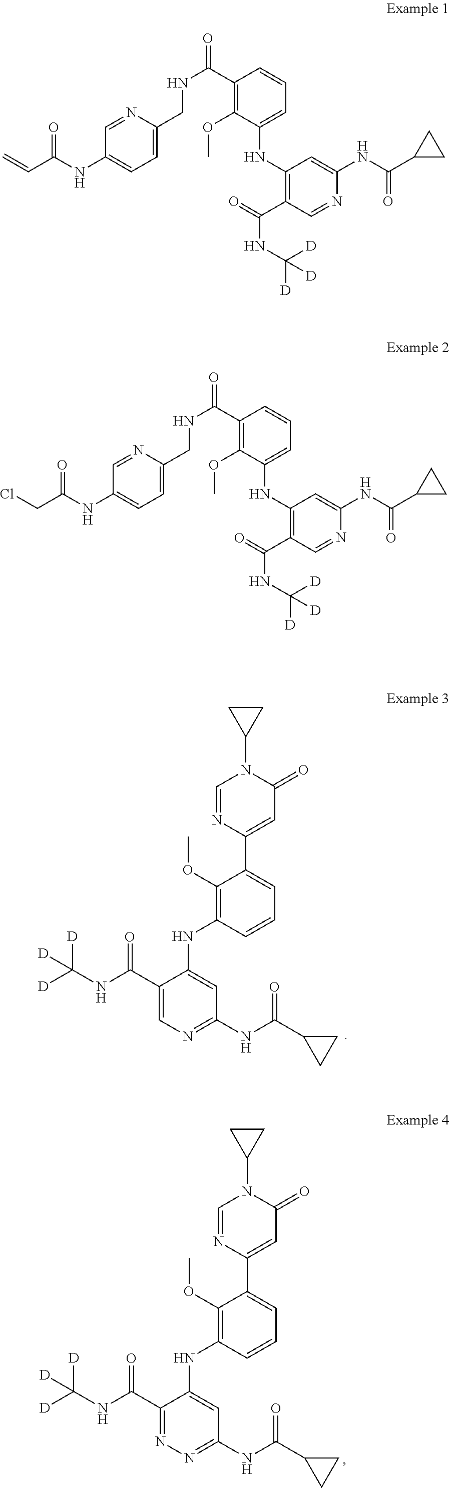 [SMILES: [2H]C([2H])([2H])NC(=O)c1cnc(NC(=O)C2CC2)cc1Nc1cccc(C(=O)NCc2ccc(NC(=O)C=C)cn2)c1OC ; [2H]C([2H])([2H])NC(=O)c1cnc(NC(=O)C2CC2)cc1Nc1cccc(C(=O)NCc2ccc(NC(=O)CCl)cn2)c1OC ; [2H]C([2H])([2H])NC(=O)c1cnc(NC(=O)C2CC2)cc1Nc1cccc(-c2cc(=O)n(C3CC3)cn2)c1OC ; [2H]C([2H])([2H])NC(=O)c1nnc(NC(=O)C2CC2)cc1Nc1cccc(-c2cc(=O)n(C3CC3)cn2)c1OC]

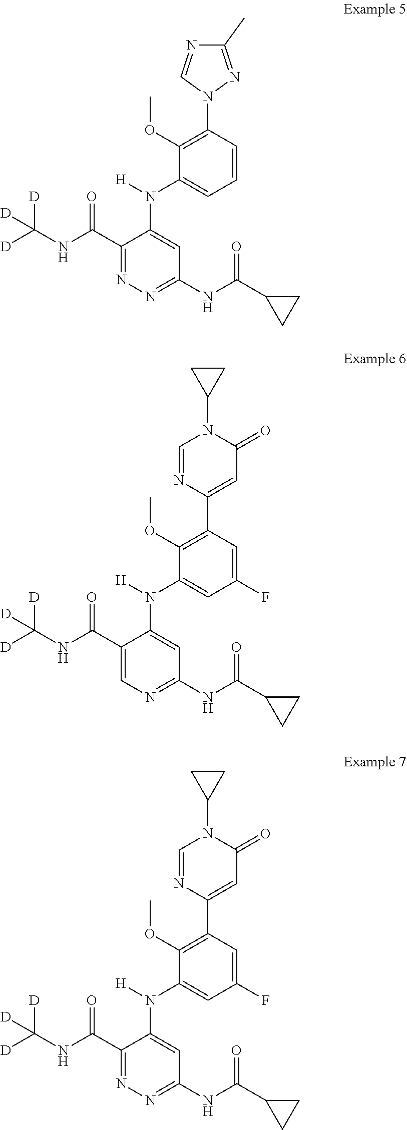 [SMILES: [2H]C([2H])([2H])NC(=O)c1nnc(NC(=O)C2CC2)cc1Nc1cccc(-n2cnc(C)n2)c1OC ; [2H]C([2H])([2H])NC(=O)c1cnc(NC(=O)C2CC2)cc1Nc1cc(F)cc(-c2cc(=O)n(C3CC3)cn2)c1OC ; [2H]C([2H])([2H])NC(=O)c1nnc(NC(=O)C2CC2)cc1Nc1cc(F)cc(-c2cc(=O)n(C3CC3)cn2)c1OC]

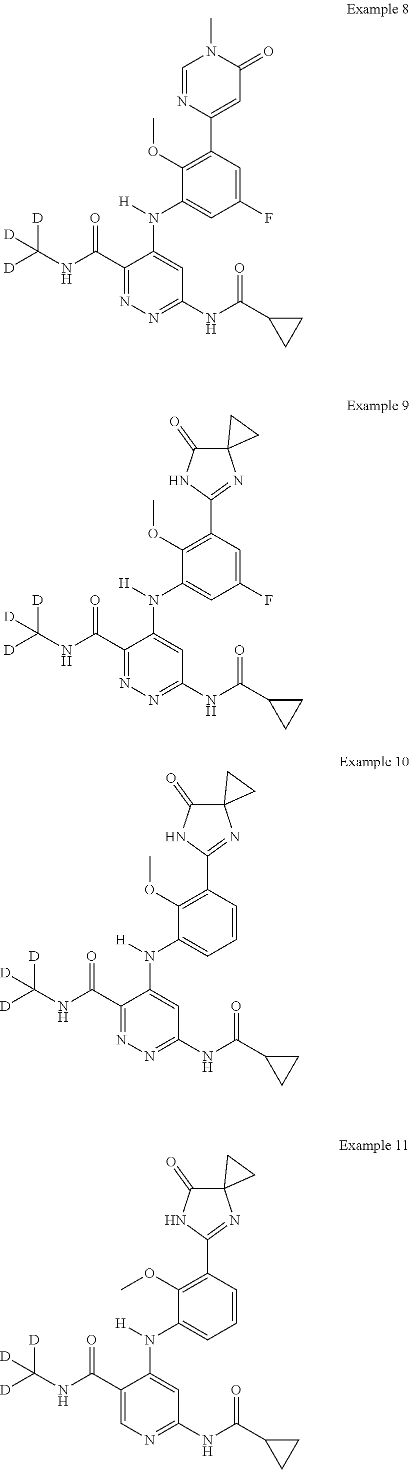 [SMILES: [2H]C([2H])([2H])NC(=O)c1nnc(NC(=O)C2CC2)cc1Nc1cc(F)cc(-c2cc(=O)n(C)cn2)c1OC ; [2H]C([2H])([2H])NC(=O)c1nnc(NC(=O)C2CC2)cc1Nc1cc(F)cc(C2=NC3(CC3)C(=O)N2)c1OC ; [2H]C([2H])([2H])NC(=O)c1nnc(NC(=O)C2CC2)cc1Nc1cccc(C2=NC3(CC3)C(=O)N2)c1OC ; [2H]C([2H])([2H])NC(=O)c1cnc(NC(=O)C2CC2)cc1Nc1cccc(C2=NC3(CC3)C(=O)N2)c1OC]

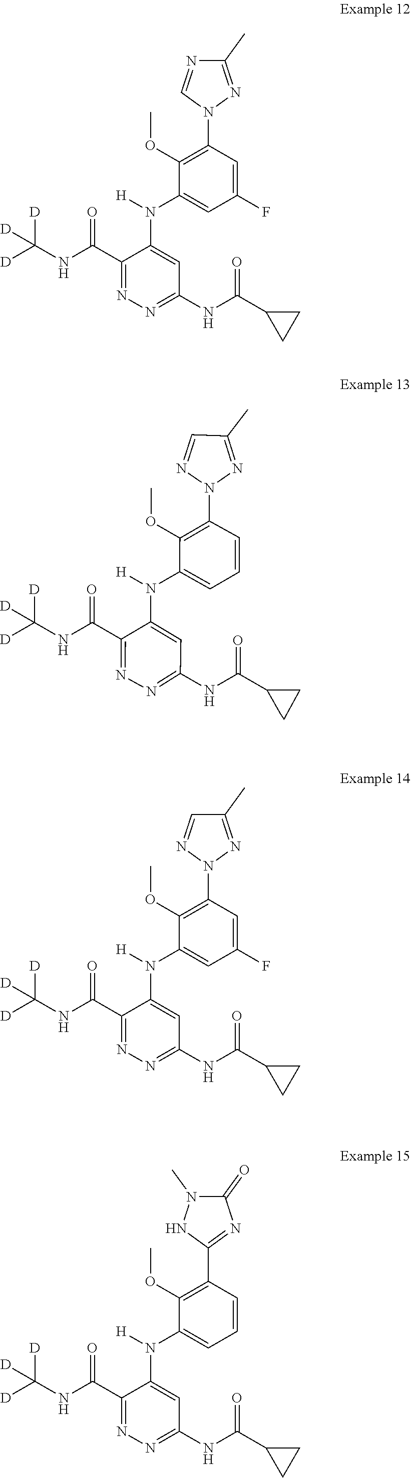 [SMILES: [2H]C([2H])([2H])NC(=O)c1nnc(NC(=O)C2CC2)cc1Nc1cc(F)cc(-n2cnc(C)n2)c1OC ; [2H]C([2H])([2H])NC(=O)c1nnc(NC(=O)C2CCC2)cc1Nc1cccc(-n2ncc(C)n2)c1OC ; [2H]C([2H])([2H])NC(=O)c1nnc(NC(=O)C2CC2)cc1Nc1cc(F)cc(-n2ncc(C)n2)c1OC ; [2H]C([2H])([2H])NC(=O)c1nnc(NC(=O)C2CC2)cc1Nc1cccc(-c2nc(=O)n(C)[nH]2)c1OC]

The compounds herein can be prepared by those skilled in the art in view of the present disclosure. Exemplary procedures are shown in the Examples section for the preparation of Examples 1-15.

In some embodiments, the present disclosure also provides a compound of Formula II

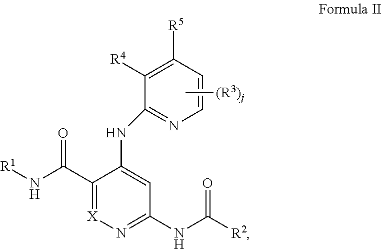

wherein the variables in Formula II can be any of those described herein in connection with Formula I or its subformulae, except that j in Formula II can only be 0, 1, or 2.

For example, in some exemplary embodiments, the present disclosure provides the following:
[1] A compound of Formula II, or a pharmaceutically acceptable salt thereof:

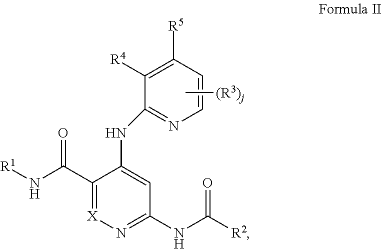

wherein:
X is CH or N;
R¹ is C₁₋₃ alkyl substituted by 0-7 deuterium atoms;
R² is optionally substituted C₁₋₆ alkyl, optionally substituted C₁₋₄ heteroalkyl, optionally substituted cycloalkyl (e.g., C₃₋₆ cycloalkyl), optionally substituted heterocyclyl (e.g., 4-8 membered heterocyclyl), or optionally substituted heteroaryl;
R³ at each occurrence is independently halogen, optionally substituted C₁₋₄ alkyl, or optionally substituted C₁₋₄ heteroalkyl;
j is 0, 1, or 2;
R⁴ is C₁₋₆ alkyl optionally substituted with 1-3 R^{A}, S(O)ₚR^{B}, or OR^{C};
   wherein:
   p is 0, 1, or 2,
   R^{A} at each occurrence is independently halogen, OH, C₁₋₆ alkyl optionally substituted with 1-3 R^{A1},
   R^{B} is C₁₋₆ alkyl optionally substituted with 1-3 R^{A1},
   R^{C} is hydrogen or C₁₋₆ alkyl optionally substituted with 1-3 R^{A2},
   wherein R^{A1} at each occurrence is independently halogen, OH, or CN; R^{A2} at each occurrence is independently F or OH;
R⁵ is

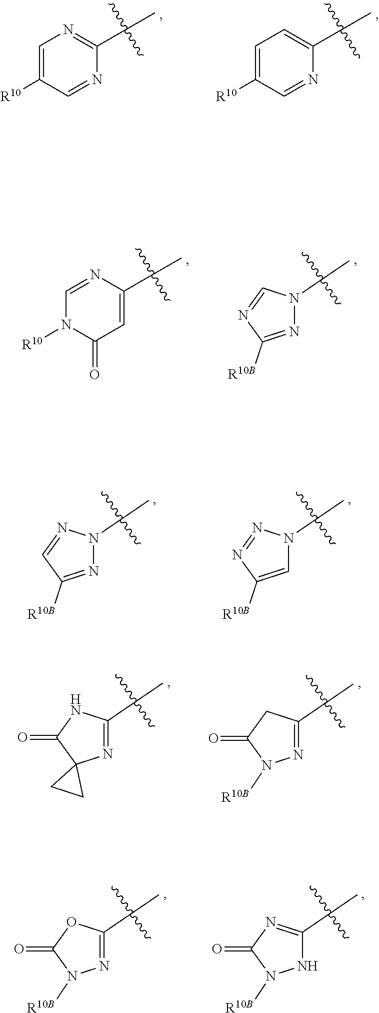

or -L¹-L²-Q-G,
wherein:
R¹⁰ at each occurrence is independently an optionally substituted cycloalkyl (e.g., C₃₋₆ cycloalkyl), or optionally substituted heterocyclyl (e.g., 4-8 membered heterocyclyl);
R^{10B} at each occurrence is independently halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl);
L¹ is O, C═(O)NH, or null,
L² is C₁₋₄ alkylene, or null,
Q is an optionally substituted heterocycle or optionally substituted heteroaryl, and
G is CN,

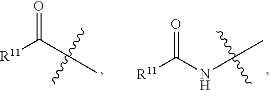

or a Michael acceptor,
wherein R¹¹ is hydrogen, an optionally substituted C₁₋₆ alkyl or an optionally substituted C₃₋₆ cycloalkyl.
[2] The compound of [1], or a pharmaceutically acceptable salt thereof, wherein X is CH.
[3] The compound of [1], or a pharmaceutically acceptable salt thereof, wherein X is N.
[4] The compound of any one of [1]-[3], or a pharmaceutically acceptable salt thereof, wherein R¹ is CH₃, C₂H₅, CD₃, or CD₂CD₃.
[5] The compound of any one of [1]-[4], or a pharmaceutically acceptable salt thereof, wherein R² is a 3-6 membered cycloalkyl, such as cyclopropyl, cyclobutyl, which is optionally substituted with 1-4 substituents independently selected from CN, halogen (e.g., F), OH, and optionally substituted C₁₋₆ alkyl.
[6] The compound of any one of [1]-[4], or a pharmaceutically acceptable salt thereof, wherein R² is cyclopropyl optionally substituted with 1-4 substituents independently selected from CN, halogen, OH, and optionally substituted C₁₋₆ alkyl.
[7] The compound of any one of [1]-[4], or a pharmaceutically acceptable salt thereof, wherein R² is cyclopropyl.
[8] The compound of any one of [1]-[7], or a pharmaceutically acceptable salt thereof, wherein j is 1.
[9] The compound of any one of [1]-[8], or a pharmaceutically acceptable salt thereof, wherein when present, R³ at each occurrence is independently F, Cl, or C₁₋₄ alkyl optionally substituted with F.
[10] The compound of any one of [1]-[7], or a pharmaceutically acceptable salt thereof, wherein j is 0.
[11] The compound of any one of [1]-[10], or a pharmaceutically acceptable salt thereof, wherein R⁴ is OMe.
[12] The compound of any one of [1]-[11], or a pharmaceutically acceptable salt thereof, wherein R⁵ is

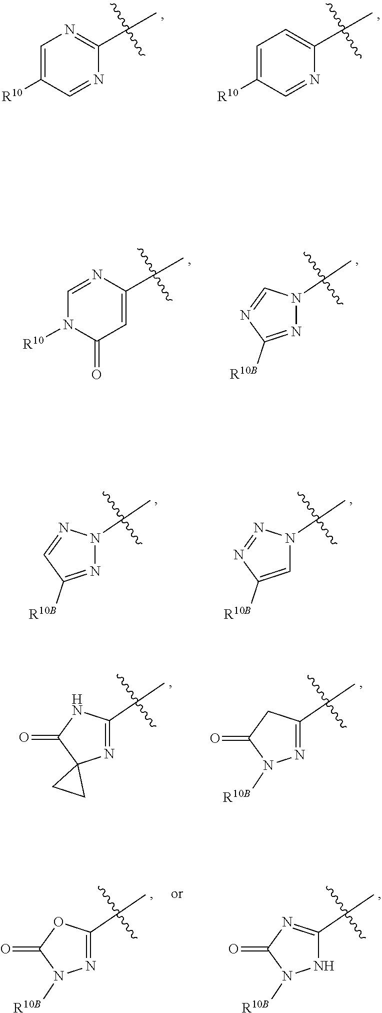

wherein R¹⁰ is an optionally substituted C₃₋₆ cycloalkyl, R^{10B} is an optionally substituted C₁₋₄ alkyl or an optionally substituted C₃₋₆ cycloalkyl.
[13] The compound of [12], or a pharmaceutically acceptable salt thereof, wherein R¹⁰ is

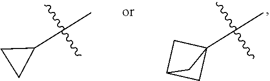

R^{10B} is methyl, CF₃, or cyclopropyl.
[14] The compound of any one of [1]-[11], or a pharmaceutically acceptable salt thereof, wherein R⁵ is -L¹-L²-Q-G.
[15] The compound of [14], or a pharmaceutically acceptable salt thereof, wherein L¹ is O.
[16] The compound of [14], or a pharmaceutically acceptable salt thereof, wherein L¹ is null.
[17] The compound of [14], or a pharmaceutically acceptable salt thereof, wherein L¹ is C═(O)NH:

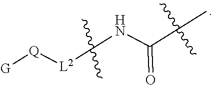

[18] The compound of any one of [14]-[17], or a pharmaceutically acceptable salt thereof, wherein L² is null.
[19] The compound of any one of [14]-[17], or a pharmaceutically acceptable salt thereof, wherein L² is C₁₋₄alkylene, e.g., CH₂.
[20] The compound of any one of [14]-[19], or a pharmaceutically acceptable salt thereof, wherein Q is a 4-7 membered monocyclic heterocyclic ring having 1 or 2 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.
[21] The compound of any one of [14]-[19], or a pharmaceutically acceptable salt thereof, wherein Q is a 4-7 membered monocyclic heterocyclic ring selected from:

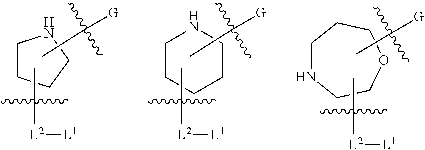

[22] The compound of any one of [14]-[19], or a pharmaceutically acceptable salt thereof, wherein Q is a 4-7 membered monocyclic heterocyclic ring selected from:

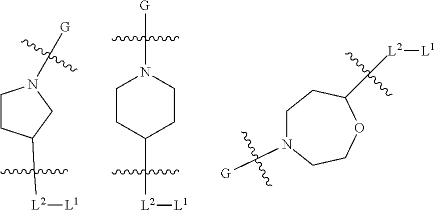

[23] The compound of any one of [14]-[19], or a pharmaceutically acceptable salt thereof, wherein Q is a 6-12 membered bicyclic heterocyclic ring having 1-4 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F,
wherein the bicyclic heterocyclic ring is a fused, spiro, or bridged bicyclic ring, wherein one of the rings is optionally aromatic or heteroaromatic.
[24] The compound of any one of [14]-[19], or a pharmaceutically acceptable salt thereof, wherein Q is a 6-10 membered bicyclic heterocyclic ring selected from:

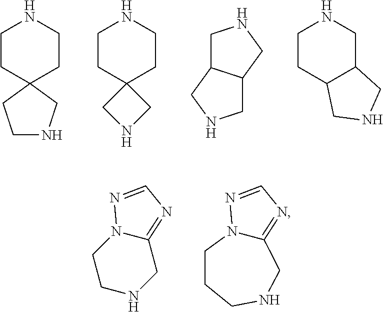

each of which is optionally substituted with one or more (e.g., 1, 2, or 3) R^{s1}, wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.
[25] The compound of any one of [14]-[19], or a pharmaceutically acceptable salt thereof, wherein Q is a 6-10 membered bicyclic heterocyclic ring selected from:

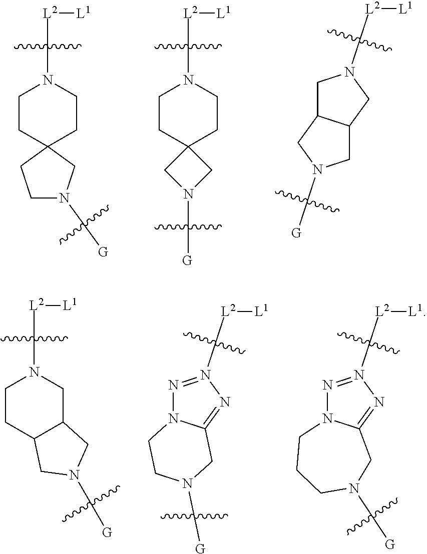

[26] The compound of any one of [14]-[19], or a pharmaceutically acceptable salt thereof, wherein Q is a 5 or 6 membered heteroaryl having 1-4 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.
[27] The compound of any one of [14]-[19], or a pharmaceutically acceptable salt thereof, wherein Q is pyridine or pyrimidine, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.
[28] The compound of any one of [14]-[19], or a pharmaceutically acceptable salt thereof, wherein Q is

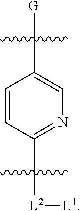

[29] The compound of any one of [14]-[28], or a pharmaceutically acceptable salt thereof, wherein G is CN.
[30] The compound of any one of [14]-[28], or a pharmaceutically acceptable salt thereof, wherein G is

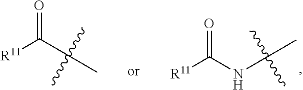

wherein R¹¹ is hydrogen or C₁₋₄ alkyl, such as methyl, optionally substituted with halogen.
[31] The compound of any one of [14]-[28], or a pharmaceutically acceptable salt thereof, wherein G is a Michael acceptor.
[32] The compound of any one of [14]-[28], or a pharmaceutically acceptable salt thereof, wherein G is

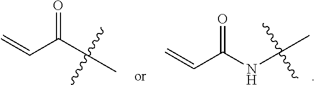

[33] The compound of any one of [1]-[11], or a pharmaceutically acceptable salt thereof, wherein R⁵ is selected from:

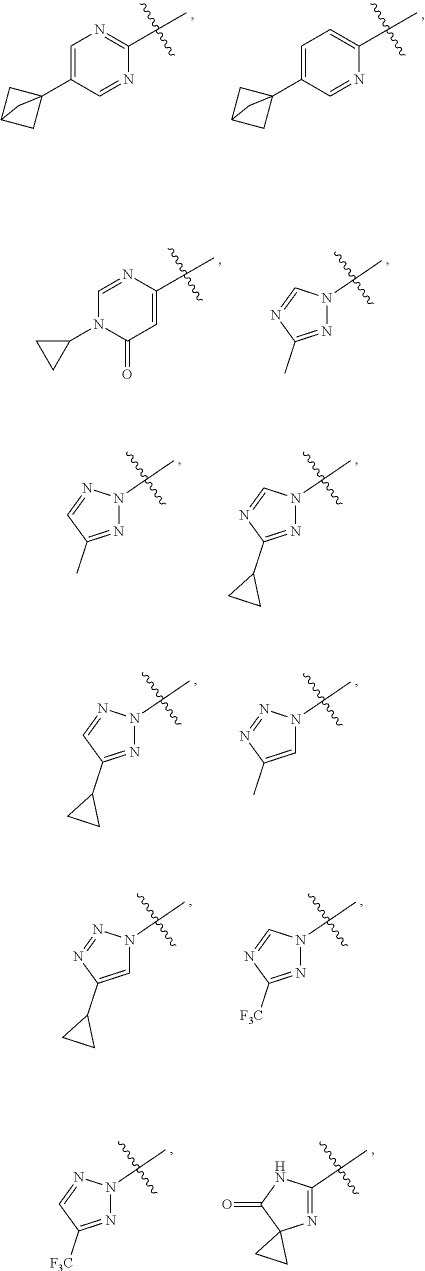

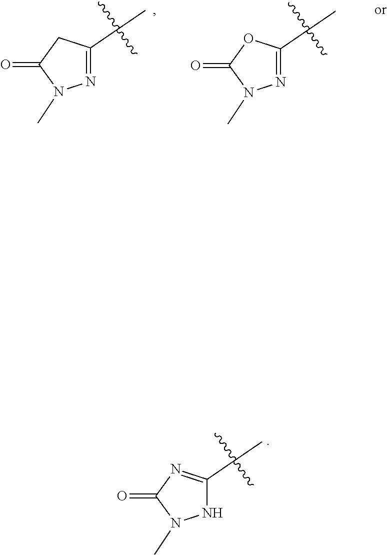

[34] The compound of any one of [1]-[11], or a pharmaceutically acceptable salt thereof, wherein R⁵ is selected from:

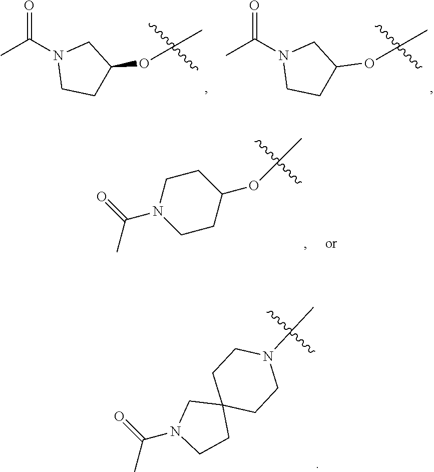

[35] The compound of any one of [1]-[11], or a pharmaceutically acceptable salt thereof, wherein R⁵ is selected from:

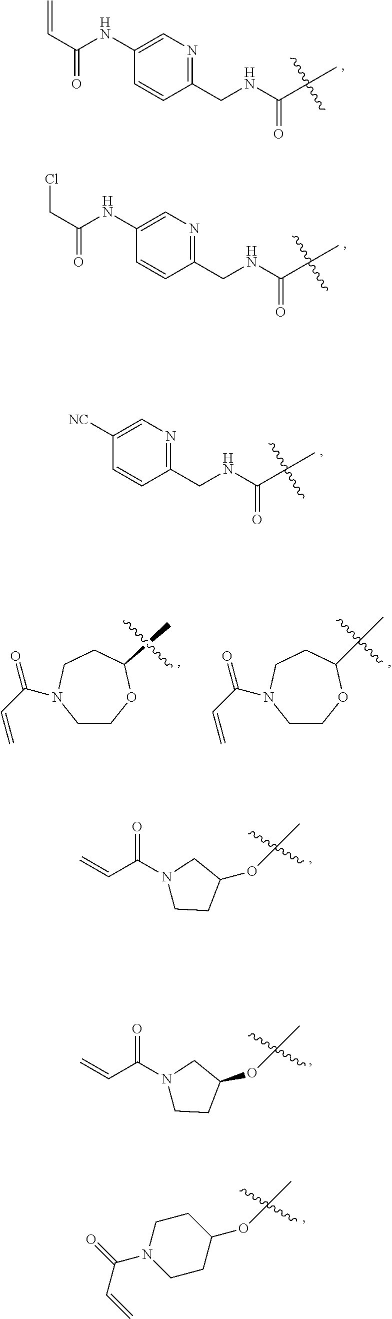

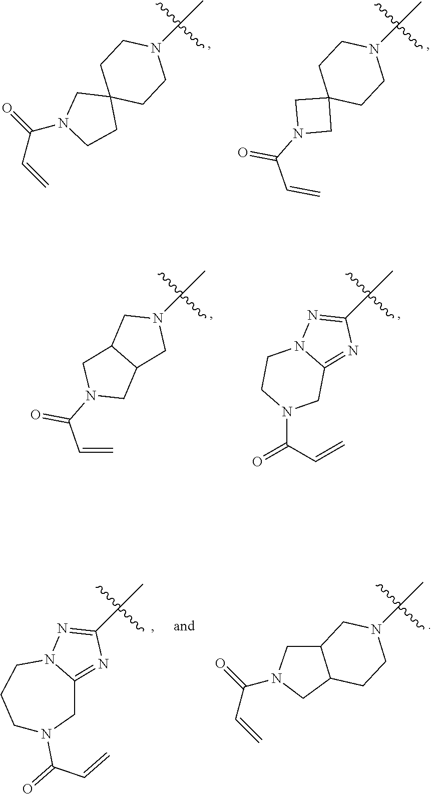 [SMILES: *N1CCC2(CC1)CCN(C(=O)C=C)C2 ; *N1CCC2(CC1)CN(C(=O)C=C)C2 ; *N1CC2CN(C(=O)C=C)CC2C1 ; *c1nc2n(n1)CCN(C(=O)C=C)C2 ; *c1nc2n(n1)CCCN(C(=O)C=C)C2 ; *N1CCC2CN(C(=O)C=C)CC2C1]

[36] A pharmaceutical composition comprising the compound of any one of [1]-[35], or a pharmaceutically acceptable salt thereof, and a pharmaceutically acceptable excipient.
[37] A method of inhibiting TYK2 in a subject or biological sample comprising contacting the subject or biological sample with an effective amount of the compound of any one of [1]-[35], or a pharmaceutically acceptable salt thereof, or the pharmaceutical composition of [36].
[38] A method of treating a TYK2-mediated disease or disorder in a subject in need thereof, the method comprising administering to the subject a therapeutically effective amount of the compound of any one of [1]-[35], or a pharmaceutically acceptable salt thereof, or the pharmaceutical composition of [36].
[39] The method of [38], wherein the TYK2-mediated disease or disorder is an autoimmune disease or disorder, an inflammatory disease or disorder, a proliferative disease or disorder, an endocrine disease or disorder (e.g., polycystic ovary syndrome, Crouzon's syndrome, or type 1 diabetes), a neurological disease or disorder (e.g., Alzheimer's disease), and/or a disease or disorder associated with transplantation (e.g., transplant rejection or graft versus host disease).
[40] The method of [38], wherein the TYK2-mediated disease or disorder is an autoimmune disease or disorder selected from type 1 diabetes, ankylosing spondylitis, cutaneous lupus erythematosus, systemic lupus erythematosus, multiple sclerosis, systemic sclerosis, psoriasis, Behçet's disease, POEMS syndrome, Crohn's disease, ulcerative colitis, inflammatory bowel disease, and combinations thereof.
[41] The method of [38], wherein the TYK2-mediated disease or disorder is an inflammatory disease or disorder selected from rheumatoid arthritis, asthma, chronic obstructive pulmonary disease, psoriasis, hepatomegaly, Crohn's disease, ulcerative colitis, inflammatory bowel disease, and combinations thereof.
[42] The method of [38], wherein the TYK2-mediated disease or disorder is a proliferative disease or disorder, such as a hematological cancer (e.g., leukemia, such as T-cell leukemia, e.g., T-cell acute lymphoblastic leukemia (T-ALL)).
[43] The method of [38], wherein the TYK2-mediated disease or disorder is associated with type I interferon, IL-10, IL-12, and/or IL-23 signaling.
[44] A method of treating psoriasis in a subject in need thereof, the method comprising administering to the subject a therapeutically effective amount of the compound of any one of [1]-[35], or a pharmaceutically acceptable salt thereof, or the pharmaceutical composition of [36].
[45] A method of treating psoriatic arthritis in a subject in need thereof, the method comprising administering to the subject a therapeutically effective amount of the compound of any one of [1]-[35], or a pharmaceutically acceptable salt thereof, or the pharmaceutical composition of [36].
[46] A method of treating systemic lupus erythematosus in a subject in need thereof, the method comprising administering to the subject a therapeutically effective amount of the compound of any one of [1]-[35], or a pharmaceutically acceptable salt thereof, or the pharmaceutical composition of [36].
[47] A method of treating Crohn's disease in a subject in need thereof, the method comprising administering to the subject a therapeutically effective amount of the compound of any one of [1]-[35], or a pharmaceutically acceptable salt thereof, or the pharmaceutical composition of [36].
[48] A method of treating ulcerative colitis in a subject in need thereof, the method comprising administering to the subject a therapeutically effective amount of the compound of any one of [1]-[35], or a pharmaceutically acceptable salt thereof, or the pharmaceutical composition of [36].
[49] A method of treating inflammatory bowel disease in a subject in need thereof, the method comprising administering to the subject a therapeutically effective amount of the compound of any one of [1]-[35], or a pharmaceutically acceptable salt thereof, or the pharmaceutical composition of [36].

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof, e.g., in a therapeutically effective amount. In any of the embodiments described herein, the pharmaceutical composition can comprise a therapeutically effective amount of a compound selected from the compounds shown in Table 1 herein, or a pharmaceutically acceptable salt thereof. In any of the embodiments described herein, the pharmaceutical composition can comprise a compound selected from:

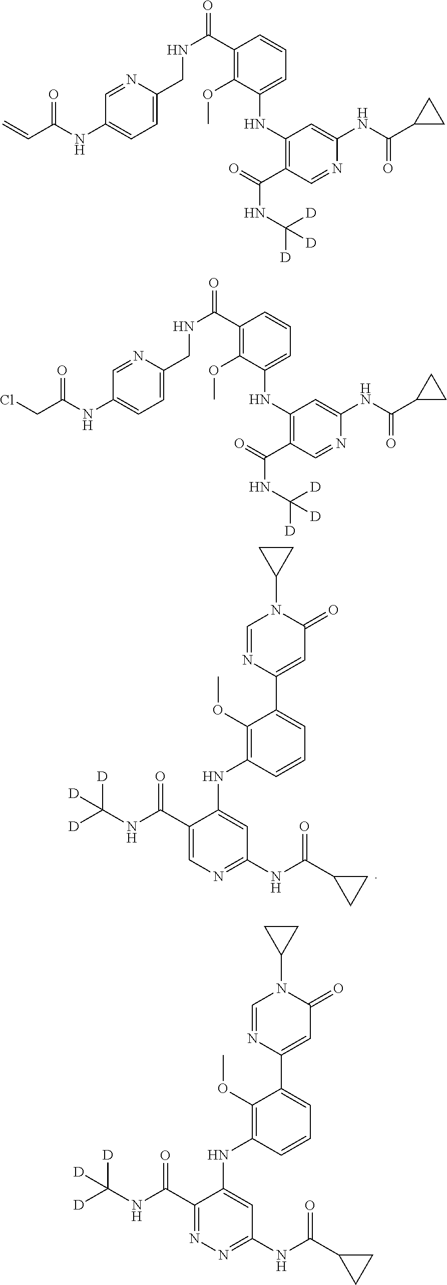

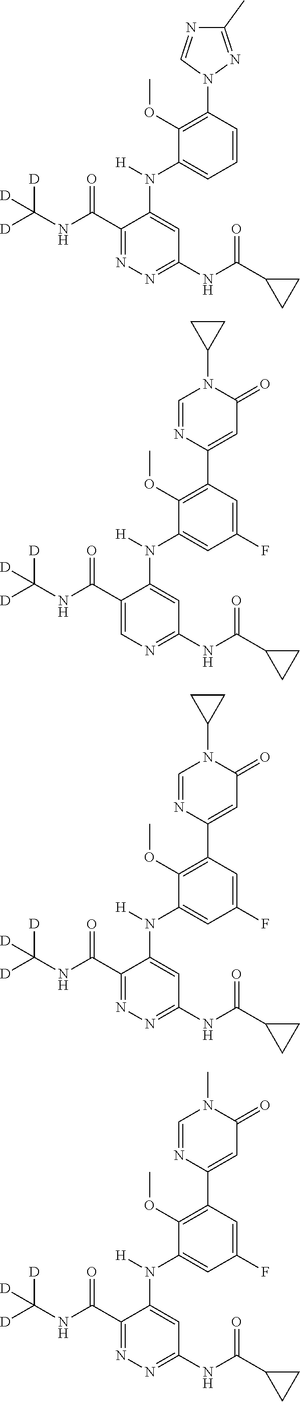

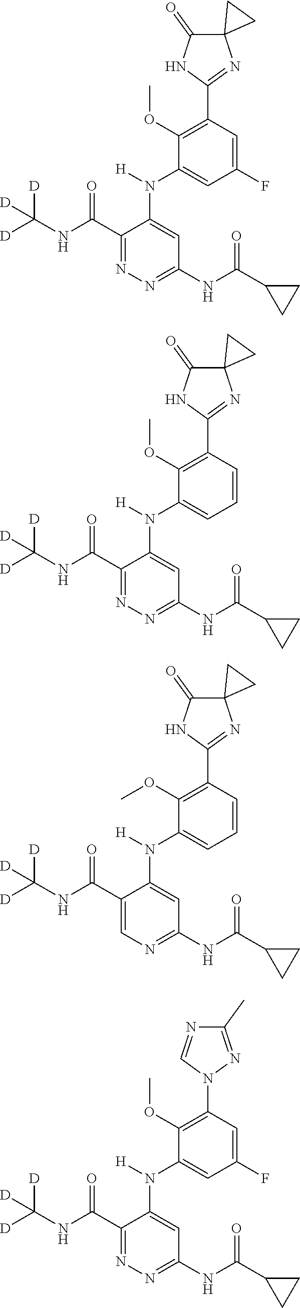

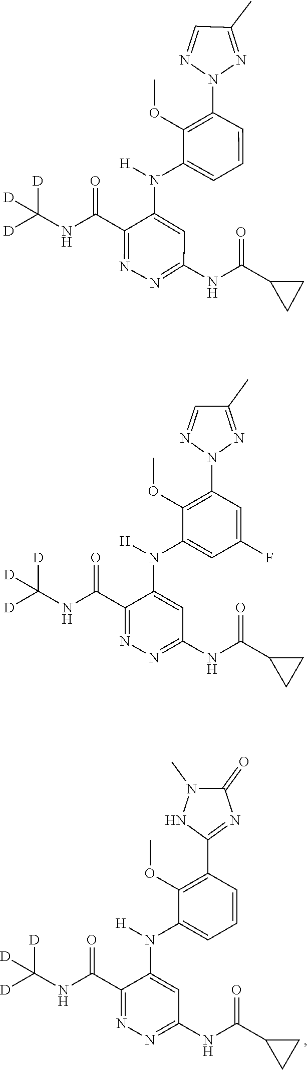 [SMILES: [2H]C([2H])([2H])NC(=O)c1nnc(NC(=O)C2CCC2)cc1Nc1cccc(-n2ncc(C)n2)c1OC ; [2H]C([2H])([2H])NC(=O)c1nnc(NC(=O)C2CC2)cc1Nc1cc(F)cc(-n2ncc(C)n2)c1OC ; [2H]C([2H])([2H])NC(=O)c1nnc(NC(=O)C2CC2)cc1Nc1cccc(-c2nc(=O)n(C)[nH]2)c1OC]

or a pharmaceutically acceptable salt thereof; typically, the compound or pharmaceutically acceptable salt thereof is present in a therapeutically effective amount for a disease or disorder described herein.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

Compounds of the present disclosure can be used alone, in combination with each other, or in combination with one or more additional therapeutic agents, e.g., an additional TYK2 inhibitor, an additional anti-inflammatory agent such as an NSAID, etc. These additional therapeutic agents include those known in the art, such as those TYK2 inhibitors and additional agents suitable for combined use with TYK2 inhibitors disclosed for example, in PCT publication Nos. WO2014/074661, WO2015/069310, WO2015/089143, WO2017/087590, WO2018/067432, WO2018/071794, WO2018/075937, WO2018/093968, WO2019/023468, WO2019/103952, WO2019/183186, WO2020/055636, WO2020/081508, WO2020/086616, WO2020/092196, WO2020/112937, WO2020/123225, and WO2020/156311, CN111909140, and U.S. Pat. Nos. 9,505,748, 10,000,480, and 10,294,256, the content of each of which is herein incorporated by reference in its entireties.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as psoriasis, psoriatic arthritis, Crohn's disease, ulcerative colitis, inflammatory bowel disease, and/or systemic lupus erythematosus, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of a TYK2-mediated disease or disorder. Accordingly, some embodiments of the present disclosure are also directed to methods of using one or more compounds of the present disclosure or pharmaceutical compositions herein for treating or preventing a TYK2-mediated disease or disorder in a subject in need thereof, such as for treating psoriasis, psoriatic arthritis, Crohn's disease, ulcerative colitis, inflammatory bowel disease, and/or systemic lupus erythematosus in a subject in need thereof.

In some embodiments, the present disclosure provides a method of inhibiting TYK2 in a subject or biological sample, which comprises contacting the subject or biological sample with an effective amount of the compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or a pharmaceutical composition described herein.

In some embodiments, the present disclosure provides a method of treating or preventing a TYK2-mediated disease or disorder in a subject in need thereof. In some embodiments, the method comprises administering to the subject an effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein. In some embodiments, the TYK2-mediated disease or disorder is associated with type I interferon, IL-10, IL-12, and/or IL-23 signaling. In some embodiments, the TYK2-mediated disease or disorder is associated with IL-12, IL-23 and/or IFNα. In some embodiments, the TYK2-mediated disease or disorder is associated with type I interferon signaling. In some embodiments, the TYK2-mediated disease or disorder is associated with IL-10 signaling. In some embodiments, the TYK2-mediated disease or disorder is associated with IL-12 signaling. In some embodiments, the TYK2-mediated disease or disorder is associated with IL-23 signaling. In some embodiments, the TYK2-mediated disease or disorder is an autoimmune disease or disorder, an inflammatory disease or disorder, a proliferative disease or disorder, an endocrine disease or disorder (e.g., polycystic ovary syndrome, Crouzon's syndrome, or type 1 diabetes), a neurological disease or disorder (e.g., Alzheimer's disease), and/or a disease or disorder associated with transplantation (e.g., transplant rejection or graft versus host disease). In some embodiments, the endocrine disease or disorder is polycystic ovary syndrome, Crouzon's syndrome, or type 1 diabetes. In some embodiments, the neurological disease or disorder is Alzheimer's disease.

In some embodiments, the present disclosure also provides a method of treating or preventing an autoimmune disease or disorder in a subject in need thereof, which comprises administering to the subject an effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein. In some embodiments, the autoimmune disease or disorder is selected from type 1 diabetes, ankylosing spondylitis, cutaneous lupus erythematosus, systemic lupus erythematosus, multiple sclerosis, systemic sclerosis, psoriasis, Behçet's disease, POEMS syndrome, Crohn's disease, ulcerative colitis, inflammatory bowel disease, and combinations thereof.

In some embodiments, the present disclosure also provides a method of treating or preventing an inflammatory disease or disorder in a subject in need thereof, which comprises administering to the subject an effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein. In some embodiments, the inflammatory disease or disorder is selected from rheumatoid arthritis, asthma, chronic obstructive pulmonary disease, psoriasis, hepatomegaly, Crohn's disease, ulcerative colitis, inflammatory bowel disease, and combinations thereof.

In some embodiments, the present disclosure also provides a method of treating or preventing proliferative disease or disorder, such as a hematological cancer (e.g., leukemia, such as T-cell leukemia, e.g., T-cell acute lymphoblastic leukemia (T-ALL)) in a subject in need thereof, which comprises administering to the subject an effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein. In some embodiments, the proliferative disease or disorder is polycythemia vera, myelofibrosis, or essential thrombocytosis. In some embodiments, the proliferative disease or disorder is a hematological cancer. In some embodiments the proliferative disease or disorder is a leukemia. In some embodiments, the leukemia is a T-cell leukemia. In some embodiments the T-cell leukemia is T-cell acute lymphoblastic leukemia (T-ALL). In some embodiments the proliferative disease or disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and/or R1027H.

In some embodiments, the present disclosure also provides a method of treating or preventing an inflammatory or allergic conditions of the skin in a subject in need thereof, which comprises administering to the subject an effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein. In some embodiments, the inflammatory or allergic conditions of the skin is selected from psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, cutaneous lupus erythematosus, systemic lupus erythematosus, pemphigus vulgaris, pemphigus foliaceus, paraneoplastic pemphigus, epidermolysis bullosa acquisita, acne vulgaris, other inflammatory or allergic conditions of the skin, and combinations thereof.

In some particular embodiments, the present disclosure also provides a method of treating psoriasis in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some particular embodiments, the present disclosure also provides a method of treating psoriatic arthritis in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some particular embodiments, the present disclosure also provides a method of treating systemic lupus erythematosus in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some particular embodiments, the present disclosure also provides a method of treating Crohn's disease in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some particular embodiments, the present disclosure also provides a method of treating ulcerative colitis in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some particular embodiments, the present disclosure also provides a method of treating inflammatory bowel disease in a subject in need thereof, which comprises administering to the subject a therapeutically effective amount of a compound of the present disclosure (e.g., a compound of Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, or a pharmaceutically acceptable salt thereof) or an effective amount of a pharmaceutical composition described herein.

In some embodiments, the compounds of the present disclosure or pharmaceutical compositions described herein can be used in treating TYK2-mediated diseases or disorders associated with IL-23, IL-12 and/or IFNα, which include, but not limited to, inflammatory diseases such as Crohn's disease, ulcerative colitis, asthma, graft versus host disease, allograft rejection, chronic obstructive pulmonary disease; autoimmune diseases such as Graves' disease, rheumatoid arthritis, systemic lupus erythematosis, cutaneous lupus, lupus nephritis, discoid lupus erythematosus, psoriasis; auto-inflammatory diseases including CAPS, TRAPS, FMF, adult onset stills, systemic onset juvenile idiopathic arthritis, gout, gouty arthritis; metabolic diseases including type 2 diabetes, atherosclerosis, myocardial infarction; destructive bone diseases or disorders such as bone resorption disease, osteoarthritis, osteoporosis, multiple myeloma-related bone disease or disorder; proliferative diseases or disorders such as acute myelogenous leukemia, chronic myelogenous leukemia; angiogenic diseases or disorders such as angiogenic diseases or disorders including solid tumors, ocular neovasculization, and infantile haemangiomas; infectious diseases such as sepsis, septic shock, and Shigellosis; neurodegenerative diseases such as Alzheimer's disease, Parkinson's disease, cerebral ischemias or neurodegenerative disease caused by traumatic injury, oncologic and viral diseases such as metastatic melanoma, Kaposi's sarcoma, multiple myeloma, and HIV infection and CMV retinitis, AIDS, respectively.

In some embodiments, the compounds of the present disclosure or pharmaceutical compositions described herein can be used in treating TYK2-mediated diseases or disorders associated with IL-23, IL-12 and/or IFNα, which include, without limitation, pancreatitis (acute or chronic), asthma, allergies, adult respiratory distress syndrome, chronic obstructive pulmonary disease, glomerulonephritis, rheumatoid arthritis, systemic lupus erythematosis, cutaneous lupus, lupus nephritis, discoid lupus erythematosus, scleroderma, chronic thyroiditis, Graves' disease, autoimmune gastritis, diabetes, autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, graft vs. host disease, inflammatory reaction induced by endotoxin, tuberculosis, atherosclerosis, muscle degeneration, cachexia, psoriatic arthritis, Reiter's syndrome, gout, traumatic arthritis, rubella arthritis, acute synovitis, pancreatic β-cell disease; diseases characterized by massive neutrophil infiltration; rheumatoid spondylitis, gouty arthritis and other arthritic conditions, cerebral malaria, chronic pulmonary inflammatory disease, silicosis, pulmonary sarcoidosis, bone resorption disease, allograft rejections, fever and myalgias due to infection, cachexia secondary to infection, keloid formation, scar tissue formation, ulcerative colitis, pyresis, influenza, osteoporosis, osteoarthritis, acute myelogenous leukemia, chronic myelogenous leukemia, metastatic melanoma, Kaposi's sarcoma, multiple myeloma, sepsis, septic shock, and Shigellosis; Alzheimer's disease, Parkinson's disease, cerebral ischemias or neurodegenerative disease caused by traumatic injury; angiogenic diseases or disorders including solid tumors, ocular neovasculization, and infantile haemangiomas; viral diseases including acute hepatitis infection (including hepatitis A, hepatitis B and hepatitis C), HIV infection and CMV retinitis, AIDS, ARC or malignancy, and herpes; stroke, myocardial ischemia, ischemia in stroke heart attacks, organ hyposia [should this be hypoxia], vascular hyperplasia, cardiac and renal reperfusion injury, thrombosis, cardiac hypertrophy, thrombin-induced platelet aggregation, endotoxemia and/or toxic shock syndrome, conditions associated with prostaglandin endoperoxidase syndase-2, and pemphigus vulgaris. In some preferred embodiments, the compounds of the present disclosure or pharmaceutical compositions described herein can be used in treating Crohn's disease, ulcerative colitis, allograft rejection, rheumatoid arthritis, psoriasis, ankylosing spondylitis, psoriatic arthritis, and/or pemphigus vulgaris. In some preferred embodiments, the compounds of the present disclosure or pharmaceutical compositions described herein can be used in treating ischemia reperfusion injury, including cerebral ischemia reperfusions injury arising from stroke and/or cardiac ischemia reperfusion injury arising from myocardial infarction. In some preferred embodiments, the compounds of the present disclosure or pharmaceutical compositions described herein can be used in treating multiple myeloma.

Diseases or disorders that can be suitably treated with the compounds, compositions, and/or methods of the present disclosure herein include any of those known diseases or disorders mediated by TYK2, some of such are disclosed for example, in PCT publication Nos. WO2014/074661, WO2015/069310, WO2015/089143, WO2017/087590, WO2018/067432, WO2018/071794, WO2018/075937, WO2018/093968, WO2019/023468, WO2019/103952, WO2019/183186, WO2020/055636, WO2020/081508, WO2020/086616, WO2020/092196, WO2020/112937, WO2020/123225, and WO2020/156311, CN111909140, and U.S. Pat. Nos. 9,505,748, 10,000,480, and 10,294,256, the content of each of which is herein incorporated by reference in its entireties.

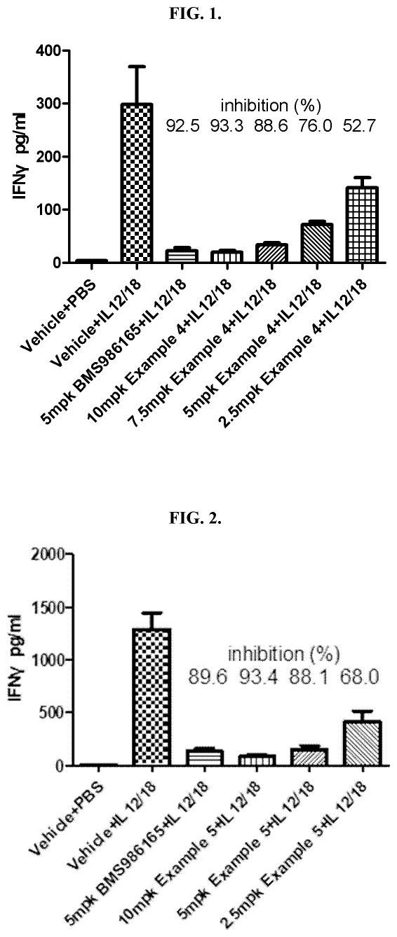

Compounds of the present disclosure can be advantageous over those known TYK2 inhibitors. Representative compounds tested herein in in vivo models were shown to have similar efficacies compared to the positive control(s) tested. For example, when tested according to Biological Example 5 herein, compound Example 5 significantly inhibited IL-12/IL-18 induced INF-gamma production at 10 mpk, greater than 93% reduction compared to vehicle treated group, with efficacy similar to the positive control tested (e.g., BMS986165). See also FIG. 2. Also tested according to Biological Example 5 herein, compound Example 4 dose-dependently inhibited IFNγ production by 52.7%, 76.0%, 88.6% and 93.3% at doses of 2.5, 5, 7.5 and 10 mpk, respectively. See also FIG. 1. In the mouse psoriasis model induced by human IL-23 herein (Biological Example 6), compounds Examples 4 and 5 were found to dose-dependently protect mice from IL-23-induced psoriasis, with efficacies comparable to the positive control tested (BMS986165).

In addition, results from functional cell based assays suggest that compounds of the present disclosure can be more selective over other JAK kinases. See Table 3 in Biological Example 4. For example, compound Example 4 was found to have an IC50 greater than 10 uM in an IFN-gamma reporter assay (JAK1/2), and compound Examples 4 and 5 were both found to have an IC50 greater than 10 uM in an IL-2 reporter assay (JAK1/3). As shown in the Examples section, in the IL-23 report assay shown herein, compound Example 4 showed an IC50 of about 18 nM and compound Example 5 showed an IC50 of about 6 nM. Compound Examples 4 and 5 were also tested in an IFNalpha/beta reporter assay (JAK1/TYK2) and were found to have an IC50 of about 4 nM and 5 nM, respectively. When tested under same assay conditions, BMS986165 was found to have an IC50 value in the INF-gamma reporter assay (JAK1/2) of 1087 nM, IL-2 reporter assay (JAK1/3) of 221 nM, IL-23 reporter assay (JAK2/TYK2) of 3.5 nM, and IFNalpha/beta reporter assay (JAK1/TYK2) of 1.3 nM. Thus, these results show that representative compounds of the present disclosure can be more selective compared to literature compounds, see e.g., Wrobleski, S. T., J. Med. Chem, 62:8973-8995 (2019).

As further shown in Biological Example 7, selected compound Example 5 was found to have a superior pharmacokinetic profile in dog compared to BMS986165 with a higher AUC and Cmax, although studies indicate that the corresponding mouse PK profiles are about the same with BMS986165. This better PK profile in combination with the better selectivity over other JAK kinases can lead to a better in vivo profile in large animals such as dogs or humans with a better safety profile.

As used herein, the term “TYK2-mediated” disorders, diseases, and/or conditions as used herein means any disease or other deleterious condition in which TYK2 or a mutant thereof is known to play a role. Such TYK2-mediated diseases or disorders include but are not limited to autoimmune diseases or disorders, inflammatory diseases or disorders, proliferative diseases or disorders, endocrine diseases or disorders, neurological diseases or disorders and diseases or disorders associated with transplantation.

The administering in the methods herein is not limited. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the administering is orally.

Compounds of the present disclosure can be used as a monotherapy or in a combination therapy. In some embodiments according to the methods described herein, compounds of the present disclosure can be administered as the only active ingredient(s). In some embodiments according to the methods described herein, compounds of the present disclosure can also be co-administered with an additional therapeutic agent, either concurrently or sequentially in any order, to the subject in need thereof.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Definitions

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I, or a subformula thereof, as applicable, are independently selected. Non-limiting useful groups for the variables in compounds of Formula I, or a subformula thereof, as applicable, include any of the respective groups, individually or in any combination, as shown in the specific compounds described in Table 1 herein or Examples 1-15. For example, in some embodiments, suitable groups as R¹ in Formula I include any of the R¹ groups shown in specific compounds described in Table 1 herein or Examples 1-15, without regard to the other variables shown in the specific compounds. In some embodiments, compounds of Formula I can include a R¹ group according to any of the R¹ groups shown in the specific compounds described in Table 1 herein or Examples 1-15 in combination at least one other variable (e.g, L¹) according to the specific compounds described in Table 1 herein or Examples 1-15, wherein the R¹ and at least one other variable can derive from the same compound or a different compound. Any such combinations are contemplated and within the scope of the present disclosure.

The described embodiments of the present disclosure can be combined. Such combination is contemplated and within the scope of the present disclosure. For example, it is contemplated that the definition(s) of any one or more of j, X, R¹, R², R³, R⁴, and R⁵ of Formula I can be combined with the definition of any one or more of the other(s) of j, X, R¹, R², R³, R⁴, and R⁵ as applicable, and the resulted compounds from the combination are within the scope of the present disclosure.

The symbol, [symbol] whether utilized as a bond or displayed perpendicular to (or otherwise crossing) a bond, indicates the point at which the displayed moiety is attached to the remainder of the molecule. It should be noted that the immediately connected group or groups maybe shown beyond the symbol, [symbol] to indicate connectivity, as would be understood by those skilled in the art.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC area, or both, or with a non-detectable amount of the other stereoisomer(s). The presence and/or amounts of stereoisomers can be determined by those skilled in the art in view of the present disclosure, including through the use of a chiral HPLC.

When a range of values is listed, it is intended to encompass each value and sub-range within the range. For example “C₁-6” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., I-1, I-2, I-1-A, I-1-A-1, I-1-A-2, I-1-A-3, I-1-A-4, I-2-A, I-2-A-1, I-2-A-2, I-2-A-3, I-2-A-4, I-1-B, I-1-B-1, I-1-B-2, I-1-B-3, I-2-B, I-2-B-1, I-2-B-2, or I-2-B-3), Formula II, or any of the compounds shown in Table 1 herein, or any of the compounds of Examples 1-15, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). The compounds of Examples 1-15 should be understood as the compounds herein labeled as Example 1, Example 2, . . . , to Example 15; it should not be confused with the compounds labeled otherwise as shown in the Examples section. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O, P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. For the avoidance of doubt, the carbocyclyl groups herein also include ring systems in which one or more rings are aryl ring(s), provided that the carbocyclyl ring as a whole is not aromatic, and the point of attachment can be on any ring. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In some embodiments, the cycloalkyl is a monocyclic ring. In some embodiments, the cycloalkyl is a fused, bridged, or spiro bicyclic C₅₋₁₀ cycloalkyl.

“Heterocyclyl” or “heterocyclic” as used by itself or as part of another group refers to a radical of a 3-membered or larger, such as 3- to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a fused, bridged, or spiro bicyclic system (“bicyclic heterocyclyl”), and can be saturated or can be partially unsaturated. Heterocyclyl bicyclic ring systems can include one or more heteroatoms in one or both rings, and the point of attachment can be on any ring. For the avoidance of doubt, the heterocyclyl groups herein include ring systems in which one or more rings are carbocyclic ring defined herein, and the point of attachment can be on any ring. In addition, the heterocyclyl groups herein also include ring systems in which one or more rings are aryl or heteroaryl ring(s), provided that the heterocyclyl ring as a whole is not a heteroaryl ring, and the point of attachment can be on any ring. As used herein, the term “heterocyclylene” as used by itself or as part of another group refers to a divalent radical derived from the heterocyclyl group defined herein. The heterocyclyl or heterocyclylene can be optionally linked to the rest of the molecule through a carbon or nitrogen atom.

Exemplary non-limiting heterocyclyl groups include azirdinyl, oxiranyl, thiiranyl, azetidinyl, oxetanyl, thietanyl, tetrahydrofuranyl, dihydrofuranyl, tetrahydrothiophenyl, dihydrothiophenyl, pyrrolidinyl, dihydropyrrolyl, pyrrolyl-2,5-dione, dioxolanyl, oxasulfuranyl, disulfuranyl, oxazolidin-2-one, triazolinyl, oxadiazolinyl, thiadiazolinyl, piperidinyl, tetrahydropyranyl, dihydropyridinyl, thianyl, piperazinyl, morpholinyl, dithianyl, dioxanyl, triazinanyl, azepanyl, oxepanyl, thiepanyl, azocanyl, oxecanyl, thiocanyl, indolinyl, isoindolinyl, dihydrobenzofuranyl, dihydrobenzothienyl, benzoxazolinonyl, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). As used herein, the term “arylene” as used by itself or as part of another group refers to a divalent radical derived from the aryl group defined herein.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein.

Exemplary non-limiting heteroaryl groups include pyrrolyl, furanyl, thiophenyl, imidazolyl, pyrazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl, triazinyl, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, purinyl, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N (except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trymethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” or “therapeutically effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. Exemplary embodiments of steps for performing the synthesis of products described herein are described in greater detail infra.

### Compound Example 1. 4-((3-(((5-acrylamidopyridin-2-yl)methyl)carbamoyl)-2-methoxyphenyl)amino)-6-(cyclopropanecarboxamido)-N-(methyl-d3)nicotinamide

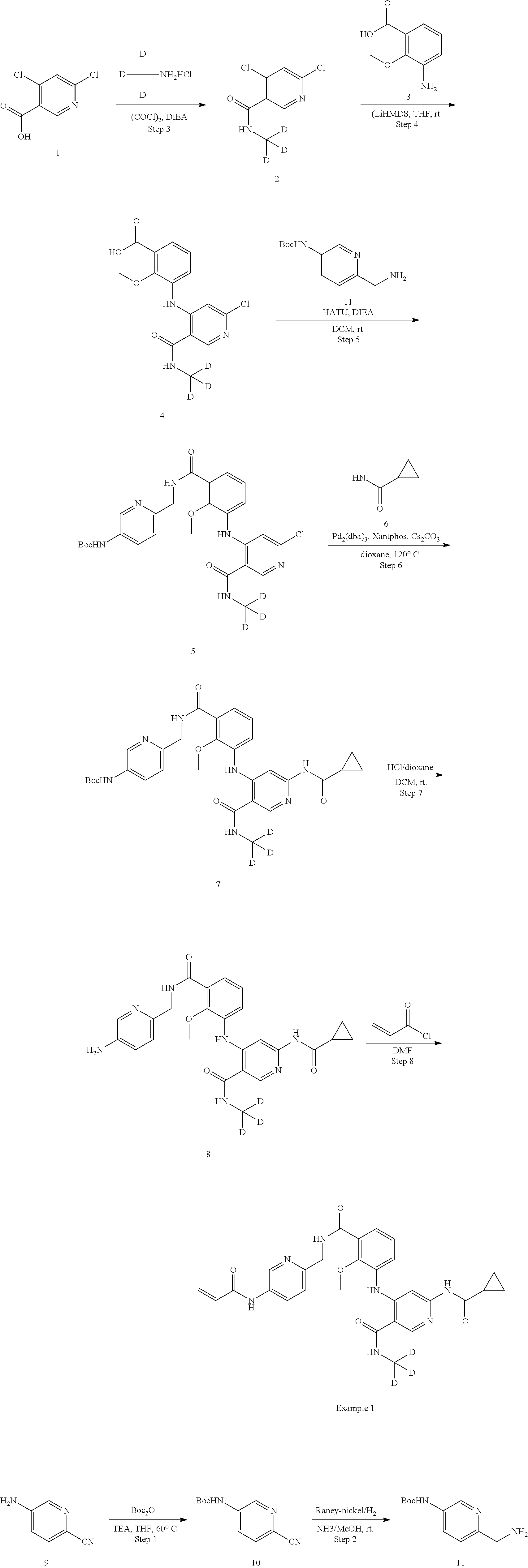

Step 1: synthesis of tert-butyl (6-cyanopyridin-3-yl)carbamate. To a solution of 5-aminopicolinonitrile (1.0 g, 8.40 mmol, 1.0 eq.), TEA (2.50 g, 25.2 mmol, 3.0 eq.) and DMAP (103 mg, 0.84 mmol, 0.1 eq.) in THF (20 mL) was added Boc₂O (2.75 g, 12.6 mmol, 1.5 eq.). The solution was stirred at 60° C. overnight and complete detected by LCMS. The solution was concentrated and purified by flash column (30-50% ethyl acetate in petroleum ether) to give tert-butyl (6-cyanopyridin-3-yl)carbamate (1.4 g, 76.1%) as yellow solid. LC-MS/ESI: m/z=220.2 [M+H]⁺.

Step 2: synthesis of tert-butyl (6-(aminomethyl)pyridin-3-yl)carbamate. To a solution of tert-butyl (6-cyanopyridin-3-yl)carbamate (1.4 g, 6.39 mmol) in NH₃/MeOH (30 mL) was added Pd/C (140 mg). The solution was stirred at rt. Overnight under H₂ and complete detected by LCMS. The mixture was filtered, the filtrate was concentrated and the residue was purified by flash column (50-100% ethyl acetate in petroleum ether) to give tert-butyl (6-(aminomethyl)pyridin-3-yl)carbamate (1.0 g, 70.1%) as yellow solid. LC-MS/ESI: m/z=224.2 [M+H]⁺

Step 3: synthesis of (E)-4-(4-methoxyphenyl)-2-(2-methyl-2H-indazol-5-yl)-8-((2-methylpropylidene) amino)-6-(2,2,2-trifluoroethoxy)pyrido[2,3-b]pyrazin-3(4H)-one. To a slurry of 4,6-dichloronicotinic acid (5 g, 20.05 mmol, 1.0 eq.) in dichloromethane (100 mL) at room temperature was added oxalyl chloride (2.96 mL, 33.85 mmol, 1.7 eq.) followed by 10 drops of DMF causing some effervescence. The mixture was stirred at room temperature for 1.5 h to form a nearly clear solution. The reaction was concentrated and the residue was dissolved in dichloroethane (30 mL) and re-concentrated and the process was repeated to ensure complete removal of the excess oxalyl chloride. The resulting crude acid chloride was dissolved in dichloromethane (100 mL) and methyl-d3-ammonium chloride (2.39 g, 33.85 mmol, 1.7 eq.) was added and the mixture was cooled in an ice bath whereupon diisopropylethylamine (13.65 mL, 78.17 mmol, 2.3 eq.) was added dropwise via syringe. After the addition was complete, the ice bath was removed and the resulting mixture was allowed to warm to room temperature and stirred overnight. The mixture was diluted with dichloromethane (100 mL) and was washed with 1 N aq. HC1 (3×100 mL) then brine before drying over anhydrous Na₂SO₄, decanting and concentrating under vacuum. This afforded 2.7 g of an off-white solid which was purified by flash column (30-50% ethyl acetate in petroleum ether) to give tert-butyl (6-(aminomethyl)pyridin-3-yl)carbamate (4.0 g, 74.1%) as white solid. LC-MS/ESI: m/z=208.2 [M+H]⁺

Step 4: synthesis of 3-((2-chloro-5-((methyl-d3)carbamoyl)pyridin-4-yl)amino)-2-methoxybenzoic acid. To a solution of 4,6-dichloro-N-(methyl-d3)nicotinamide (900 mg, 4.35 mmol, 1.0 eq.) and 3-amino-2-methoxybenzoic acid (900 mg, 4.35 mmol, 1.0 eq.) in THF (20 mL) was added dropwise LiHMDS (4.35 mL, 1M in THF) at rt. The solution was stirred at rt. for 2 hours and complete detected by LCMS. The solution was quenched with aqueous HCl solution (10 mL, 0.5 mol/L), extracted with EA (20 mL*3). The organic layer was washed with brine (5 mL), dried over anhydrous sodium sulfate, concentrated and purified by flash column (50-100% ethyl acetate in petroleum ether) to give 3-((2-chloro-5-((methyl-d3)carbamoyl)pyridin-4-yl)amino)-2-methoxybenzoic acid (650 mg, 40.8% yield) as yellow solid. LC-MS/ESI: m/z=339.2 [M+H]⁺

Step 5: synthesis of tert-butyl (6-((3-((2-chloro-5-((methyl-d3)carbamoyl)pyridin-4-yl)amino)-2-methoxybenzamido)methyl)pyridin-3-yl)carbamate. To a solution of 3-((2-chloro-5-((methyl-d3)carbamoyl)pyridin-4-yl)amino)-2-methoxybenzoic acid (600 mg, 1.78 mmol, 1 eq.), TEA (539 mg, 5.33 mmol, 3 eq.) in DCM (10 mL) was added HATU (720 mg, 1.87 mmol, 1.05 eq.). The solution was stirred at rt for 0.5 h and then tert-butyl (6-(aminomethyl)pyridin-3-yl)carbamate (417 mg, 1.87 mmol, 1.05 eq.) was added to the reaction. The solution was stirred at rt for 2 h and complete detected by LCMS. The solution was concentrated and purified by flash column (50-70% ethyl acetate in petroleum ether) to give tert-butyl (6-((3-((2-chloro-5-((methyl-d3)carbamoyl)pyridin-4-yl)amino)-2-methoxybenzamido)methyl)pyridin-3-yl)carbamate (900 mg, 93.4%) as yellow solid. LC-MS/ESI: m/z=544.2 [M+H]⁺

Step 6: synthesis of tert-butyl (6-((3-((2-(cyclopropanecarboxamido)-5-((methyl-d3)carbamoyl) pyridine-4-yl)amino)-2-methoxybenzamido)methyl)pyridin-3-yl)carbamate. To a solution of tert-butyl (6-((3-((2-chloro-5-((methyl-d3)carbamoyl)pyridin-4-yl)amino)-2-methoxybenzamido)methyl)pyridin-3-yl)carbamate (450 mg, 0.83 mmol, 1 eq.), cyclopropanecarboxamide (141 mg, 1.66 mmol, 2 eq.), Cs₂CO₃ (822 mg, 2.52 mmol, 3 eq.) Xantphos (96 mg, 0.17 mmol, 0.2 eq.) in dioxane (20 mL) was added Pd₂(dba)₃ (76 mg, 0.083 mmol, 0.1 eq.). The solution was stirred at 120° C. overnight under N₂ and complete detected by LCMS. The solution was concentrated and purified by flash column (70-100% ethyl acetate in petroleum ether) to give tert-butyl (6-((3-((2-(cyclopropanecarboxamido)-5-((methyl-d3)carbamoyl)pyridin-4-yl)amino)-2-methoxybenzamido)methyl)pyridin-3-yl)carbamate (360 mg, 73.4%) as yellow solid. LC-MS/ESI: m/z=593.3 [M+H]⁺

Step 7: synthesis of 4-((3-(((5-aminopyridin-2-yl)methyl)carbamoyl)-2-methoxyphenyl)amino)-6-(cyclopropanecarboxamido)-N-(methyl-d3)nicotinamide. To a solution of tert-butyl (6-((3-((2-(cyclopropanecarboxamido)-5-((methyl-d3)carbamoyl)pyridin-4-yl)amino)-2-methoxybenzamido)methyl)pyridin-3-yl) carbamate (360 mg, 0.61 mmol) in DCM (10 mL) was added HCl/dioxane (10 mL, 4M/L). The solution was stirred at rt. for 3 h and complete detected by LCMS. The mixture was dissolved in DCM (15 mL) and neutralized with NaHCO_{3(aq.)} (10 mL, 2N) extracted with DCM (20 mL*3). The organic layer was washed with brine (5 mL), dried over anhydrous sodium sulfate, concentrated and the residue was purified by flash column (5-10% MeOH in DCM) to give 4-((3-(((5-aminopyridin-2-yl)methyl)carbamoyl)-2-methoxyphenyl)amino)-6-(cyclopropanecarboxamido)-N-(methyl-d3)nicotinamide (250 mg, 83.5%) as yellow solid. LC-MS/ESI: m/z=493.2 [M+H]⁺

Step 8: synthesis of Example 1, 4-((3-(((5-acrylamidopyridin-2-yl)methyl)carbamoyl)-2-methoxyphenyl)amino)-6-(cyclopropanecarboxamido)-N-(methyl-d3)nicotinamide. To a solution of 4-((3-(((5-aminopyridin-2-yl)methyl)carbamoyl)-2-methoxyphenyl)amino)-6-(cyclopropanecarboxamido)-N-(methyl-d3)nicotinamide (50 mg, 0.10 mmol, 1.0 eq.) in DCM (1 mL) and DMF (1 mL) was added acryloyl chloride (10 mg, 0.11 mmol, 1.1 eq.). The solution was stirred at 0° C. for 3 h and complete detected by LCMS. The mixture was neutralized with NaHCO_{3(aq.)} (10 mL, 2N) extracted with EA (20 mL*3). The organic layer was washed with brine (5 mL), dried over anhydrous sodium sulfate, concentrated and the residue was purified by Pre-HPLC to give 4-((3-(((5-acrylamidopyridin-2-yl)methyl)carbamoyl)-2-methoxyphenyl)amino)-6-(cyclopropanecarboxamido)-N-(methyl-d3)nicotinamide (30 mg, 54.1%) as white solid. LC-MS/ESI: m/z=545.3 [M−H]⁻, ¹HNMR (400 MHz, DMSO) δ 10.84 (s, 1H), 10.71 (s, 1H), 10.38 (s, 1H), 8.96 (t, J=5.8 Hz, 1H), 8.79 (d, J=2.3 Hz, 1H), 8.64 (s, 1H), 8.52 (s, 1H), 8.10 (dd, J=8.5, 2.4 Hz, 1H), 7.97 (s, 1H), 7.55 (d, J=7.9 Hz, 1H), 7.40 (d, J=8.3 Hz, 2H), 7.25 (t, J=7.9 Hz, 1H), 6.45 (dd, J=17.0, 10.1 Hz, 1H), 6.29 (dd, J=17.0, 1.9 Hz, 1H), 5.80 (dd, J=10.1, 1.8 Hz, 1H), 4.56 (d, J=5.7 Hz, 2H), 3.73 (s, 3H), 2.01-1.93 (m, 1H), 0.78 (d, J=6.1 Hz, 4H).

### Compound Example 2. 4-((3-(((5-(2-chloroacetamido)pyridin-2-yl)methyl)carbamoyl)-2-methoxyphenyl)amino)-6-(cyclopropanecarboxamido)-N-(methyl-d3)nicotinamide

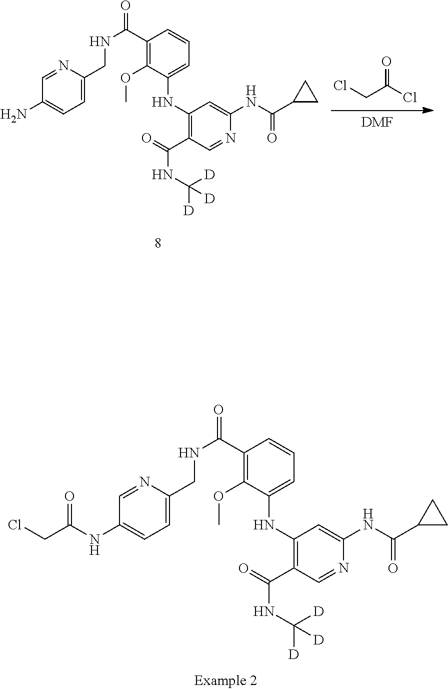

To a solution of 4-((3-(((5-aminopyridin-2-yl)methyl)carbamoyl)-2-methoxyphenyl)amino)-6-(cyclopropanecarboxamido)-N-(methyl-d3)nicotinamide (100 mg, 0.20 mmol, 1.0 eq.) in DCM (2 mL) and DMF (2 mL) was added 2-chloroacetyl chloride (25 mg, 0.22 mmol, 1.1 eq.). The solution was stirred at 0° C. for 3 h and complete detected by LCMS. The mixture was neutralized with aqueous NaHCO₃ solution (10 mL, 2N), extracted with EA (20 mL*3). The combined organic layers were washed with brine (5 mL), dried over anhydrous sodium sulfate, concentrated and the residue was purified by Pre-HPLC to give 4-((3-(((5-(2-chloroacetamido)pyridin-2-yl)methyl)carbamoyl)-2-methoxyphenyl)amino)-6-(cyclopropanecarboxamido)-N-(methyl-d3)nicotinamide (40 mg, 34.6%) as white solid. LC-MS/ESI: m/z=567.2 [M−H]⁻, ¹HNMR (400 MHz, DMSO) δ 10.78 (s, 1H), 10.69 (s, 1H), 10.52 (s, 1H), 8.96 (t, J=5.8 Hz, 1H), 8.71 (d, J=2.3 Hz, 1H), 8.61 (s, 1H), 8.53 (s, 1H), 8.03 (s, 1H), 8.02 (dd, J=7.9, 2.3 Hz, 1H), 7.55 (dd, J=7.9, 1.3 Hz, 1H), 7.38 (dd, J=11.5, 5.0 Hz, 2H), 7.24 (t, J=7.9 Hz, 1H), 4.56 (d, J=5.8 Hz, 2H), 4.30 (s, 2H), 3.73 (s, 3H), 2.03-1.92 (m, 1H), 0.77 (d, J=5.9 Hz, 4H).

### Compound Example 3. 6-(cyclopropanecarboxamido)-4-((3-(1-cyclopropyl-6-oxo-1,6-dihydropyrimidin-4-yl)-2-methoxyphenyl)amino)-N-(methyl-d3)nicotinamide

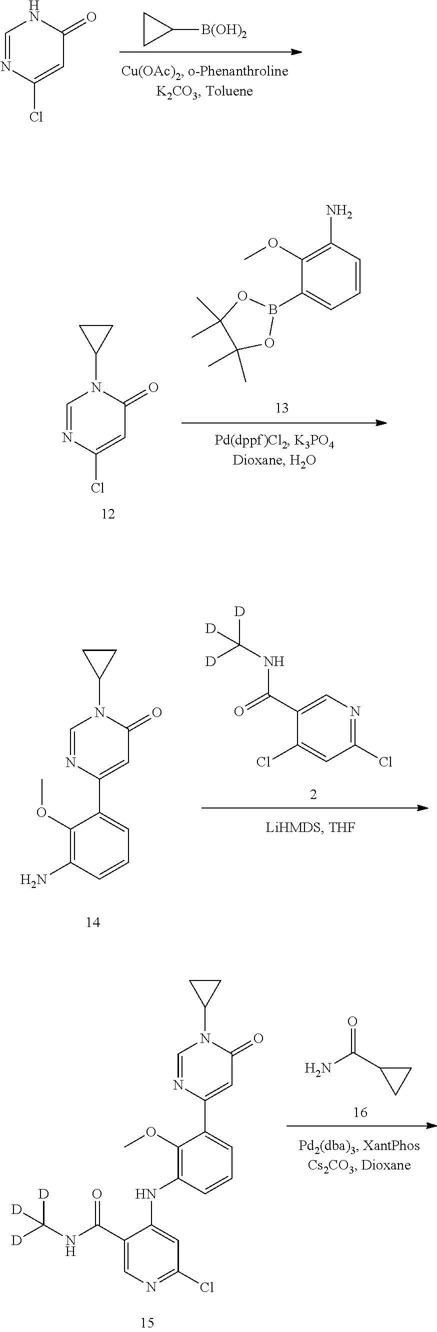

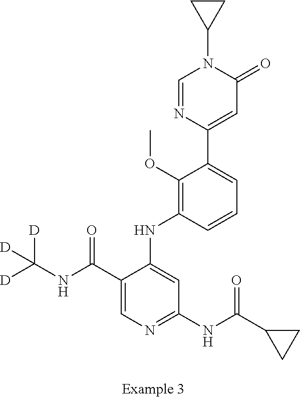 [SMILES: [2H]C([2H])([2H])NC(=O)c1cnc(NC(=O)C2CC2)cc1Nc1cccc(-c2cc(=O)n(C3CC3)cn2)c1OC]

Step 1: Synthesis of compound 12. To a solution of 6-chloropyrimidin-4(3H)-one (1.00 g, 7.66 mmol) in toluene (30 mL) were added cyclopropyl boronic acid (1.32 g, 15.32 mmol), K₂CO₃ (2.12 g, 15.32 mmol), o-Phenanthroline (180.2 mg, 0.99 mmol) and Cu(OAc)₂ (181.9 mg, 0.99 mmol) at room temperature. The mixture was stirred at 70° C. for 17 hr under O₂ (15 psi) atmosphere. After the reaction was completed, the resulting mixture was diluted with ethyl acetate (60 mL) and filtered after stirred for 5 mins. The filtrate was washed with water (30 mL) and brine (30 mL) successively, dried over anhydrous Na₂SO₄, and then evaporated under vacuum to dryness after filtration. The residue was purified by chromatography on silica gel (Petroleum ether:Ethyl acetate=100:1 to 10:1) to give compound 12 (464 mg, 35.50%) as white solid. LC/MS (ESI) m/z: 171 (M+H)⁺.

Step 2: Synthesis of compound 14. To a solution of compound 12 (100.0 mg, 0.586 mmol) in dioxane (24 mL) and H₂O (3 mL) were added compound 13 (146.0 mg, 0.586 mmol), K₃PO₄ (623.5 mg, 2.930 mmol), Pd(dppf)Cl₂ (65.1 mg, 0.09 mmol) at room temperature. The mixture was stirred at 130° C. for 3 hr under N₂ atmosphere. The resulting mixture was diluted with ethyl acetate (50 mL) and filtered after cooled to room temperature. The filtrate was washed with water (25 mL) and brine (25 mL) successively, dried over anhydrous Na₂SO₄, and concentrated under vacuum to dryness after filtration. The residue was purified by chromatography on silica gel (DCM:MeOH=100:1 to 100:5) to give compound 14 (124 mg, 82.22%) as yellow oil. LC/MS (ESI) m/z: 258 (M+H)⁺.

Step 3: Synthesis of compound 15. To a solution of compound 14 (124 mg, 0.48 mmol) and 2 (100 mg, 0.48 mmol) in THF (10 mL) was added 1 M LiHMDS/THF (1.45 mL, 1.44 mmol) solution during 5 mins. The mixture was stirred at room temperature for 1.5 h before quenched by slow addition of water (5 mL). The resulting mixture was adjusted with 1N HCl aqueous solution to pH 9-10, and then extracted with ethyl acetate (20 mL*2). The combined organic layers were washed with water (15 mL) and brine (15 mL) successively, dried over anhydrous Na₂SO₄, and concentrated under vacuum to dryness after filtration. The residue was purified by chromatography on silica gel (DCM:MeOH=100:1 to 100:5) to give compound 15 (67.0 mg, 32.41%) as white solid. LC/MS (ESI) m/z: 429 (M+H)⁺.

Step 4: Synthesis of Example 3. To a solution of compound 15 (67.0 mg, 0.16 mmol) in dioxane (5 mL) were added 16 (39.88 mg, 0.47 mmol), Cs₂CO₃ (101.8 mg, 0.31 mmol), Pd₂(dba)₃ (14.2 mg, 0.02 mmol) and XantPhos (18.2 mg, 0.02 mmol) at room temperature. The mixture was stirred at 130° C. for 7 hr under N₂ atmosphere. The resulting mixture was diluted with ethyl acetate (20 mL) and filtered after cooled to room temperature. The filtrate was washed with water (20 mL) and brine (20 mL) successively, dried over anhydrous Na₂SO₄, and concentrated under vacuum to dryness after filtration. The residue was purified by pre-HPLC (Column: YMC Triart C18 250*20 mm I.D, 5 um, Flow: 14 ml/min, Gradient: 0% to 55% acetonitrile in H₂O with 0.1% FA in H₂O) to give Example 3 which is 6-(cyclopropanecarboxamido)-4-((3-(1-cyclopropyl-6-oxo-1,6-dihydropyrimidin-4-yl)-2-methoxyphenyl)amino)-N-(methyl-d3)nicotinamide (13 mg, 17.43%) as white solid. LC/MS (ESI) m/z: 478 (M+H)⁺. ¹H-NMR (400 MHz, MeOD) δ 8.45 (s, 1H), 8.40 (s, 1H), 8.02 (s, 1H), 7.61 (s, 1H), 7.59 (s, 1H), 7.27 (t, J=8.0 Hz, 1H), 7.07 (d, J=0.6 Hz, 1H), 3.67 (s, 3H), 3.28 (dd, J=7.5, 4.2 Hz, 1H), 1.89-1.80 (m, 1H), 1.18 (q, J=7.4 Hz, 2H), 1.05-0.99 (m, 2H), 0.94 (dt, J=7.7, 3.9 Hz, 2H), 0.87 (ddd, J=10.8, 6.9, 3.9 Hz, 2H).

### Compound Example 4. 6-(cyclopropanecarboxamido)-4-((3-(1-cyclopropyl-6-oxo-1,6-dihydropyrimidin-4-yl)-2-methoxyphenyl)amino)-N-(methyl-d3)pyridazine-3-carboxamide

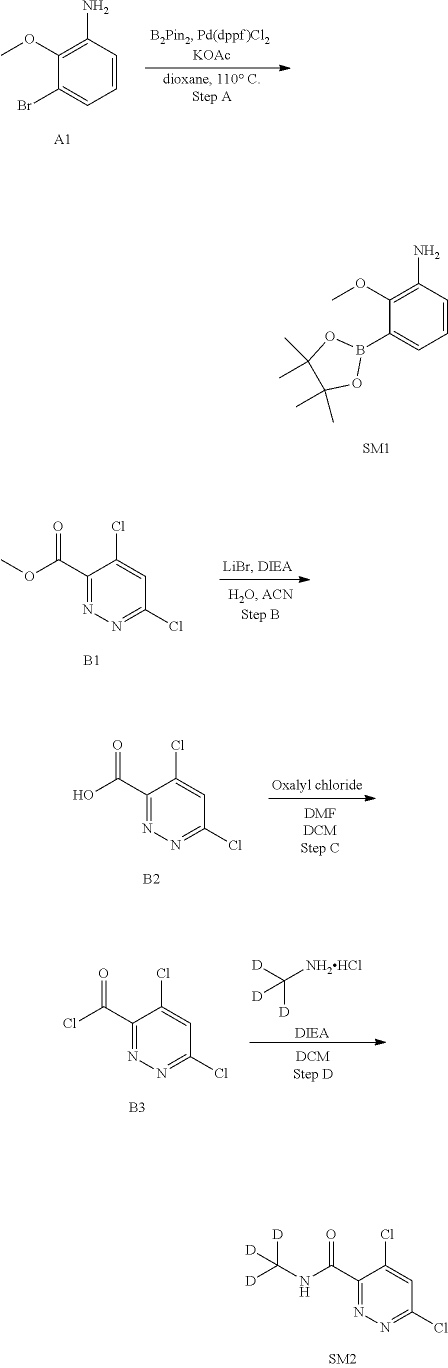

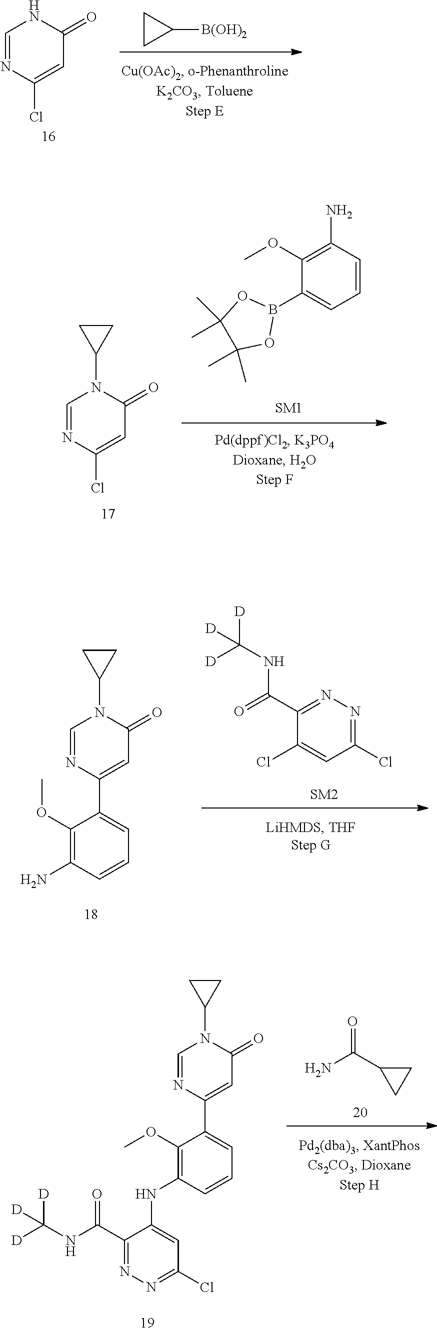

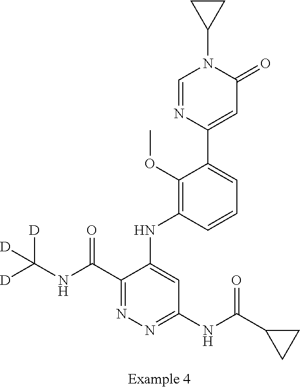 [SMILES: [2H]C([2H])([2H])NC(=O)c1nnc(NC(=O)C2CC2)cc1Nc1cccc(-c2cc(=O)n(C3CC3)cn2)c1OC]

Step A: Synthesis of SM1. A mixture of 3-bromo-2-methoxyaniline A1 (6.0 g, 29.7 mmol), 4,4,4′,4′,5,5,5′,5′-octamethyl-2,2′-bi(1,3,2-dioxaborolane) (11.2 g, 44.54 mmol), Pd(dppf)Cl₂ (1.45 g, 1.78 mmol), and potassium acetate (8.7 g, 89 mmol) in 1,4-dioxane (100 mL) was heated in a pressure bottle at 110° C. for 20 hrs. Upon cooling to rt, the mixture was diluted with ethyl acetate (100 mL) and filtered through Celite. The filtrate was concentrated under vacuum, and the residue was subjected to flash chromatography (0-40% ethyl acetate/hexane) to provide SM1 (5.4 g, 22.9 mmol, 77% yield) as a white solid. LC-MS (ESI) m/z 250 [M+H]⁺.

Step B: Synthesis of B2. LiBr (9.23 g, 106.28 mmol, 1.1 eq.), H₂O (8 mL) and acetonitrile (60 mL) were charged to a glass bottle stirred at room temperature. methyl 4,6-dichloropyridazine-3-carboxylate B1 (20 g, 96.61 mmol, 1 eq.) was added. Followed by DIEA (14.98 g, 115.94 mmol, 1.2 eq.). The reaction was stirred for 3 h, then the precipitate was filtered and washed with acetonitrile (4 mL) and dried by residue vacuo to give B2 (14.9 g, 79.9%) as a white solid. LC-MS (ESI): m/z 191, 193 [M−H]⁻.

Step C: Synthesis of B3. B2 (15 g, 77.73 mmol, 1 eq.) and DMF (568 mg, 7.77 mmol, 0.1 eq.) was added in DCM (150 mL) under nitrogen. Then Oxalyl chloride (11.84 g, 93.27 mmol, 1.2 eq.) was added dropwise to the mixture at 0° C. The reaction was stirred at room temperature for 2 hrs. The solvent was removed under reduce pressure, then DCM (100 mL) was added. The DCM was removed to give 4,6-dichloropyridazine-3-carbonyl chloride (15.4, 93.7%). LC-MS (ESI): m/z 207.1 [M+MeOH]⁺.

Step D: Synthesis of SM2. A solution of 4,6-dichloropyridazine-3-carbonyl chloride (15.4 g, 72.84 mmol, 1.1 eq.), and DIEA (8.56 g, 66.22 mmol, 2.0 eq.) in DCM (150 mL). Methan-d3-amine hydrochloride (4.67 g, 66.22 mmol, 1 eq.) was added to the mixture and the reaction was stirred at room temperature for 2 h. Then the mixture was slowly added to 0.5N HCl and the organic layer was washed with water the brine. The organic layer was dried by Na2SO4 and concentrated. The residue was chromatographed to give 4,6-dichloro-N-(methyl-d3) pyridazine-3-carboxamide SM2 (8.6 g, 62.1%). ¹H NMR (400 MHz, DMSO-d₆): δ 8.90 (s, 1H), 8.48 (s, 1H). LC-MS (ESI): m/z 206.1 [M+H]⁺.

Step E: Synthesis of 17. To a solution of 16 (10.0 g, 76.6 mmol) in toluene (300 mL) were added cyclopropyl boronic acid (13.2 g, 153.2 mmol), K₂CO₃ (21.2 g, 153.2 mmol), o-Phenanthroline (1.80 g, 9.9 mmol) and Cu(OAc)₂ (1.82 g, 9.9 mmol) at room temperature. The mixture was stirred at 70° C. for 17 hr under O₂ (15 psi) atmosphere. After the reaction was completed, the resulting mixture was diluted with ethyl acetate (600 mL) and filtered after stirred for 5 mins. The filtrate was washed with water (300 mL) and brine (300 mL) successively, dried over anhydrous Na₂SO₄, and then evaporated under vacuum to dryness after filtration. The residue was purified by chromatography on silica gel (Petroleum ether:Ethyl acetate=100:1 to 10:1) to give 17 (3.03 g, 23.5%) as white solid. LC/MS (ESI) m/z: 171 [M+H]⁺.

Step F: Synthesis of 18. To a solution of 17 (2.40 g, 14.07 mmol) in dioxane (320 mL) and H₂O (40 mL) were added SM1 (3.86 g, 15.48 mmol), K₃PO₄ (14.93 g, 70.34 mmol), Pd(dppf)Cl₂ (1.54 g, 2.11 mmol) at room temperature. The mixture was stirred at 120° C. for 4 hrs under N₂ atmosphere. The resulting mixture was diluted with ethyl acetate (50 mL) and filtered after cooled to room temperature. The filtrate was washed with water (25 mL) and brine (25 mL) successively, dried over anhydrous Na₂SO₄, and concentrated under vacuum to dryness after filtration. The residue was purified by chromatography on silica gel (DCM:MeOH=100:1 to 100:5) to give 18 (3.10 g, 85.64%) as yellow oil. LC/MS (ESI) m/z: 258 (M+H)⁺.

Step G: Synthesis of 19. To a solution of 18 (3.10 g, 12.05 mmol) and SM2 (2.52 g, 0.48 mmol) in THF (150 mL) was added 1 M LiHMDS/THF (36.15 mL, 250.85 mmol) solution during 15 mins. The mixture was stirred at rt for 1.5 h before quenched by slow addition of water (50 mL). The resulting mixture was adjusted with 1N HCl aqueous solution to pH 9-10, and then extracted with ethyl acetate (25 mL×2). The combined organic layers were washed with water (20 mL) and brine (20 mL) successively, dried over anhydrous Na₂SO₄, and concentrated under vacuum to dryness after filtration. The residue was purified by chromatography on silica gel (DCM:MeOH=100:1 to 100:5) to give 19 (4.28 g, 82.63%) as yellow solid. LC/MS (ESI) m/z: 429 [M+H]⁺.

Step H: Synthesis of Example 4. To a solution of 19 (4.20 g, 9.77 mmol) in dioxane (70 mL) were added cyclopropanecarboxamide 20 (914.6 mg, 10.75 mmol), Cs₂CO₃ (6.37 g, 19.54 mmol), Pd₂(dba)₃ (894.7 mg, 0.97 mmol) and XantPhos (1.13 g, 1.95 mmol) at room temperature. The mixture was stirred at 140° C. for 2 hrs under microwave irradiation (100 w) at CEM microwave reactor. The resulting mixture was diluted with ethyl acetate (50 mL) and filtered after cooled to room temperature. The filtrate was washed with water (25 mL) and brine (25 mL) successively, dried over anhydrous Na₂SO₄, and concentrated under vacuum to dryness after filtration. The residue was purified by chromatography on silica gel (DCM:MeOH=100:1 to 100:5) to give crude solid (2.4 g) as yellow solid. MeOH (10 mL) was added to the residue and the resulting slurry was filtered after stirred for 30 mins. The filter cake was washed with MeOH (2 mL) and dried under vacuum to give pure Example 4 (1.74 g, 37.22%). ¹H-NMR (400 MHz, DMSO-d₆) δ 11.33 (s, 1H), 10.96 (s, 1H), 9.15 (s, 1H), 8.48 (s, 1H), 8.13 (s, 1H), 7.62 (d, J=7.8 Hz, 1H), 7.55 (d, J=7.7 Hz, 1H), 7.31 (t, J=7.9 Hz, 1H), 6.87 (s, 1H), 3.60 (s, 3H), 3.29-3.22 (m, 1H), 2.13-2.03 (m, 1H), 1.07-0.96 (m, 4H), 0.82 (d, J=4.6 Hz, 4H). LC-MS (ESI) m/z: 478 [M+H]⁺.

### Compound Example 5. 6-(cyclopropanecarboxamido)-4-((2-methoxy-3-(3-methyl-1H-1,2,4-triazol-1-yl)phenyl)amino)-N-(methyl-d3)pyridazine-3-carboxamide

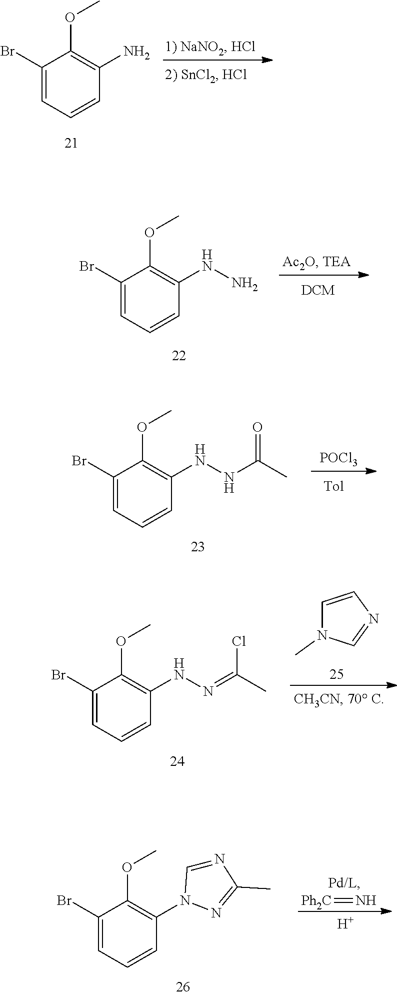

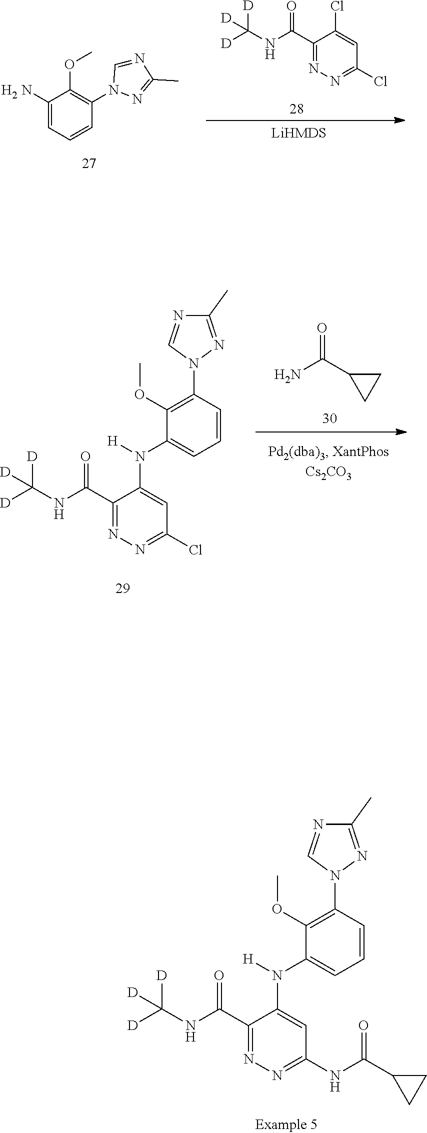

Step 1: Synthesis of compound 22. To a solution of 300 mL HCl (18%, aqueous) were added 21 (65 g, 323.3 mmol) at 0° C. Then NaNO₂ (33.5 g, 485 mmol) dissolved in 400 mL H₂O was added dropwise into the mixture at 0° C. and stirred for 1 h at 0° C. Then a solution of SnCl₂ (123 g, 647 mmol) in 120 mL concentrated HCl was added dropwise to the mixture at 0° C. (Be careful due to the reaction is exothermic). After completion, the mixture was warm to room temperature and stirred for 2 h. The mixture was filtered and the solid was washed with a small amount of water. The solid was dissolved in 300 mL saturated K₂CO₃ solution and 400 mL EtOAc. The organic layer was separated and the water layer was extracted with EtOAc (200 mL×2). The organic layer was combined, dried over anhydrous Na₂SO₄, and then evaporated under vacuum. The residue was purified by column chromatography on silica gel eluted with PE/EtOAc (2:1˜1:1) to give 22 (40 g, 57.1%) as white solid. LC/MS (ESI) m/z: 217, 219 [M+H]⁺.

Step 2: Synthesis of 23. To a solution of 22 (40.0 g, 184.2 mmol) in DCM (300 mL) were added TEA (37.3 g, 368.5 mmol), Ac₂O (28.2 g, 276.4 mmol) at 0° C. The mixture was stirred at room temperature for 3 hr under N₂ atmosphere. After which, the resulting mixture was poured into ice water (300 mL) and extracted with DCM (300 mL*2), dried over anhydrous Na₂SO₄, and then evaporated under vacuum. The residue was purified by chromatography on silica gel (Petroleum ether:EtOAc=10:1 to 0:1) to give pure 23 (22 g, 46.1%) as white solid. LC/MS (ESI) m/z: 259,261 (M+H)⁺.

Step 3: Synthesis of 24. To a solution of 23 (8 g, 30.9 mmol) in Tol (150 mL) were added POCl₃ (4.97 g, 32.4 mmol) at room temperature. The mixture was stirred at 80° C. for 2 hrs in a sealed tube. After which, the resulting mixture was concentrated under vacuum. The residue was purified by chromatography on silica gel (Petroleum ether) to give compound 24 (5.8 g, 67.7%) as yellow oil.

Step 4: Synthesis of 26. A solution of 24 (5.8 g, 20.9 mmol) and 1-methyl-1H-imidazole (4.3 g, 52.2 mmol) in MeCN (180 mL) was stirred at 90° C. for 15 hrs, added DIPEA (8.1 g, 62.7 mmol) and DMSO (90 mL), stirred at 100° C. for additional 15 hrs. After which, the resulting mixture was poured into ice water (200 mL) and extracted with EtOAc (200 mL*3), washed with water (300 mL) and brine (300 mL), dried over anhydrous Na₂SO₄, and then evaporated under vacuum. The residue was purified by chromatography on silica gel (Petroleum ether:Ethyl acetate=10:1 to 1:1) to give 26 (2.1 g, 37.5%) as brown oil. LC/MS (ESI) m/z: 268,270 [M+H]⁺.

Step 5: Synthesis of 27. A solution of 26 (2.1 g, 7.9 mmol) and Ph₂C═NH (1.72 g, 9.5 mmol) in dioxane (20 mL) was added Pd₂(dba)₃ (201 mg, 0.35 mmol), Xantphos (300, 0.52 mmol) and Cs₂CO₃ (7.7 g, 23.6 mmol). The mixture was sealed and stirred at 120° C. for 12 hrs. The mixture was filtered and concentrated to dryness. Then the residue was added into a solution of 20 mL THF and 5 mL HCl (18%, aqueous). The mixture was stirred for 4 h. The mixture was basified with 15 mL saturated K₂CO₃ and extracted with 20 mL EtOAc for three times. The organic layer was combined, dried over anhydrous Na₂SO₄, and then evaporated under vacuum. The residue was purified by column chromatography on silica gel eluted with PE/EtOAc (3:1˜2:1) to give 27 (1.1 g, 68.8%) as a white solid. LC/MS (ESI) m/z: 205 [M+H]⁺.

Step 6: Synthesis of compound 29. To a solution of 27 (1.1 g, 5.4 mmol) and 28 (1.35 g, 6.48 mmol) in THF (20 mL) were added LiHMDS (12.4 mL, 12.4 mmol) at 0° C. The mixture was stirred at room temperature for 2 hrs under N₂ atmosphere. After which, the resulting mixture was poured into saturated NH₄Cl (20 mL) and extracted with EtOAc (30 mL*3), dried over anhydrous Na₂SO₄, and then evaporated under vacuum. The residue was purified by chromatography on silica gel (Petroleum ether:Ethyl acetate=3:1 to 2:1) to give 29 (1.8 g, 88.7%) as a white solid. LC/MS (ESI) m/z: 377.0 [M+H]⁺.

Step 7: Synthesis of Example 5. A solution of 29 (1.5 g, 4.0 mmol) and cyclopropanecarboxamide 30 (408 mg, 4.8 mmol) in dioxane (20 mL) was added Pd₂(dba)₃ (201 mg, 0.35 mmol), Xantphos (300, 0.52 mmol) and Cs₂CO₃ (3.9 g, 12 mmol). The mixture was sealed and stirred at 135° C. for 12 hrs. The mixture was filtered and concentrated to dryness. The residue was purified by column chromatography on silica gel eluted with CH₂Cl₂/MeOH (4:1˜2:1) to give pure Example 5 (0.9 g, 53.0%). ¹H NMR (400 MHz, DMSO-d₆): δ11.37 (s, 1H), 11.05 (s, 1H), 9.17 (s, 1H), 8.82 (s, 1H), 8.18 (s, 1H), 7.54 (d, 1H), 7.46 (d, 1H), 7.35 (t, 1H), 3.55 (s, 3H), 2.38 (s, 3H), 2.15-2.05 (m, 1H), 0.83 (s, 4H). LC-MS (ESI): m/z 426 [M+H]⁺.

### Compound Examples 6-15

The following examples were prepared according to similar procedures as shown above in Examples 1-5. Representative characterization data are shown in the table below:

| Example | | | LCMS |
|---|---|---|---|
| No. | Structure | NMR data | (M + H)⁺ |
| 6 | 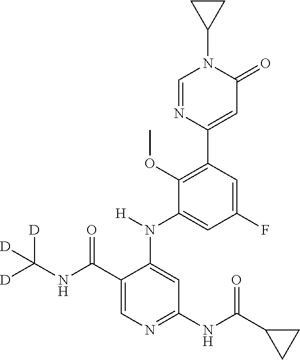 [SMILES: [2H]C([2H])([2H])NC(=O)c1cnc(NC(=O)C2CC2)cc1Nc1cc(F)cc(-c2cc(=O)n(C3CC3)cn2)c1OC] | 1H NMR (400 MHz, MeOD) δ 8.46 (s, 1H), 8.43 (s, 1H), 8.11 (s, 1H), 7.37 (ddd, J = 12.5, 9.4, 3.1 Hz, 2H), 7.16 (d, J = 0.5 Hz, 1H), 3.66 (s, 3H), 3.27 (dd, J = 7.4, 4.1 Hz, 1H), 1.90-1.83 (m, 1H), 1.18 (q, J = 7.1 Hz, 2H), 1.04-0.96 (m, 4H), 0.91- 0.85 (m, 2H). | 496.3 |
| 7 | 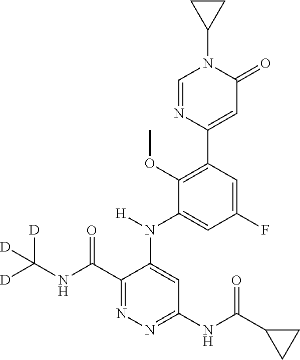 [SMILES: [2H]C([2H])([2H])NC(=O)c1nnc(NC(=O)C2CC2)cc1Nc1cc(F)cc(-c2cc(=O)n(C3CC3)cn2)c1OC] | ¹H NMR (400 MHz, DMSO) δ 11.40 (s, 1H), 11.13 (s, 1H), 9.19 (s, 1H), 8.49 (s, 1H), 8.22 (s, 1H), 7.47 (dd, J = 9.4, 3.1 Hz, 1H), 7.38 (dd, J = 9.6, 3.1 Hz, 1H), 6.94 (s, 1H), 3.59 (s, 3H), 2.07-2.11 (m, 1H), 1.23 (s, 1H), 1.08-0.95 (m, 4H), 0.84 (d, J = 6.1 Hz, 4H). | 497.3 |
| 8 | 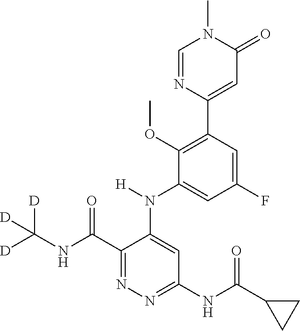 [SMILES: [2H]C([2H])([2H])NC(=O)c1nnc(NC(=O)C2CC2)cc1Nc1cc(F)cc(-c2cc(=O)n(C)cn2)c1OC] | 1H NMR (400 MHz, DMSO) δ 11.40 (s, 1H), 11.14 (s, 1H), 9.19 (s, 1H), 8.60 (s, 1H), 8.22 (s, 1H), 7.47 (dd, J = 9.5, 3.1 Hz, 1H), 7.38 (dd, J = 9.5, 3.1 Hz, 1H), 6.95 (s, 1H), 3.59 (s, 3H), 3.46 (s, 3H), 2.14-2.08 (m, 1H), 0.85 (d, J = 6.1 Hz, 4H). | 471.1 |
| 9 | 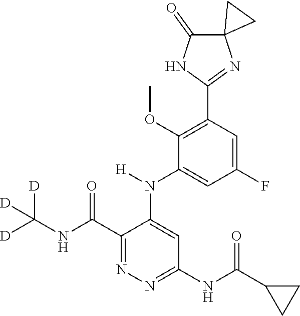 [SMILES: [2H]C([2H])([2H])NC(=O)c1nnc(NC(=O)C2CC2)cc1Nc1cc(F)cc(C2=NC3(CC3)C(=O)N2)c1OC] | 1H NMR (400 MHz, DMSO) δ 11.43 (s, 1H), 11.31 (s, 1H), 11.21 (s, 1H), 9.21 (s, 1H), 8.25 (s, 1H), 7.57 (dd, J = 9.6, 2.9 Hz, 1H), 7.31 (dd, J = 8.9, 2.9 Hz, 1H), 3.70 (s, 3H), 2.09 (dd, J = 12.2, 6.2 Hz, 1H), 1.75 (dd, J = 8.1, 4.1 Hz, 2H), 1.53 (dd, J = 8.1, 4.1 Hz, 2H), 0.86 (d, J = 6.0 Hz, 4H). | 471.2 |
| 10 | 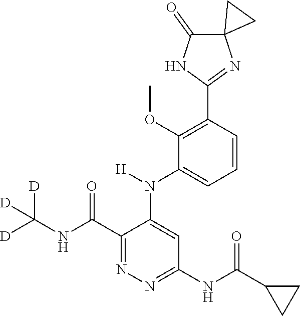 [SMILES: [2H]C([2H])([2H])NC(=O)c1nnc(NC(=O)C2CC2)cc1Nc1cccc(C2=NC3(CC3)C(=O)N2)c1OC] | 1H NMR (400 MHz, DMSO) δ11.35 (s, 1H), 11.18 (s, 1H), 9.15 (s, 1H), 8.14 (s, 1H), 7.63 (d, J = 7.8 Hz, 1H), 7.53 (d, J = 7.6 Hz, 1H), 7.31 (t, J = 7.8 Hz, 1H), 3.70 (s, 3H), 2.08 (s, 1H), 1.72 (d, J = 3.4 Hz, 2H), 1.51 (d, J = 3.4 Hz, 2H), 0.82 (s, 4H). | 453.3 |
| 11 | 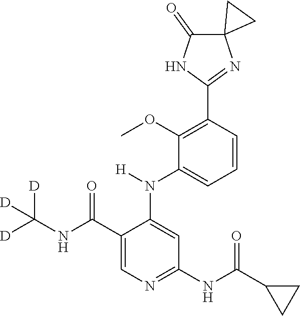 [SMILES: [2H]C([2H])([2H])NC(=O)c1cnc(NC(=O)C2CC2)cc1Nc1cccc(C2=NC3(CC3)C(=O)N2)c1OC] | 1H NMR (400 MHz, DMSO) δ11.14 (s, 1H), 10.80 (s, 1H), 10.72 (s, 1H), 8.61 (s, 1H), 8.53 (s, 1H), 8.04 (s, 1H), 7.60 (d, J = 7.7 Hz, 1H), 7.44 (d, J = 7.6 Hz, 1H), 7.27 (t, J = 7.9 Hz, 1H), 3.69 (s, 3H), 1.97 (dd, J = 12.2, 6.2 Hz, 1H), 1.71 (dd, J = 8.1, 4.1 Hz, 2H), 1.49 (dd, J = 8.0, 4.0 Hz, 2H), 0.78 (d, J = 4.8 Hz, 4H). | 452.3 |
| 12 | 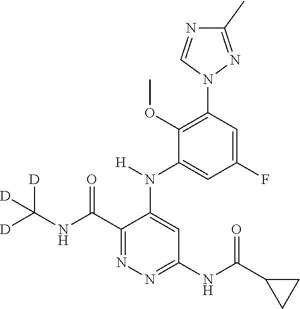 [SMILES: [2H]C([2H])([2H])NC(=O)c1nnc(NC(=O)C2CC2)cc1Nc1cc(F)cc(-n2cnc(C)n2)c1OC] | ¹H NMR (400 MHz, DMSO) δ 11.44 (s, 1H), 11.25 (s, 1H), 9.22 (s, 1H), 8.90 (s, 1H), 8.27 (s, 1H), 7.46 (dd, J = 9.7, 2.9 Hz, 1H), 7.33 (dd, J = 9.1, 2.9 Hz, 1H), 3.55 (s, 3H), 2.38 (s, 3H), 2.10 (dd, J = 12.3, 6.2 Hz, 1H), 0.85 (d, J = 6.1 Hz, 4H). | 444.2 |
| 13 | 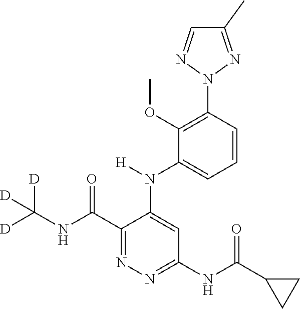 [SMILES: [2H]C([2H])([2H])NC(=O)c1nnc(NC(=O)C2CC2)cc1Nc1cccc(-n2ncc(C)n2)c1OC] | 1H NMR (400 MHz, DMSO) δ 11.36 (s, 1H), 11.03 (s, 1H), 9.16 (s, 1H), 8.17 (s, 1H), 7.91 (s, 1H), 7.59 (dd, J = 7.7, 1.2 Hz, 1H), 7.40-7.31 (m, 2H), 3.59 (s, 3H), 2.37 (s, 3H), 2.08 (dd, J = 12.1, 6.2 Hz, 1H), 0.83 (d, J = 5.7 Hz, 4H). | 426.3 |
| 14 | 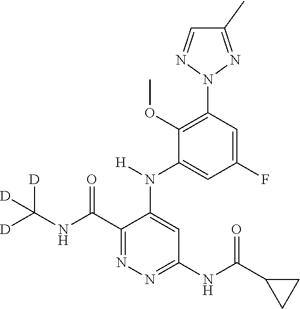 [SMILES: [2H]C([2H])([2H])NC(=O)c1nnc(NC(=O)C2CC2)cc1Nc1cc(F)cc(-n2ncc(C)n2)c1OC] | 1H NMR (400 MHz, MeOD) δ 8.37 (s, 1H), 7.78 (s, 1H), 7.43 (dd, J = 9.7, 2.9 Hz, 1H), 7.21 (dd, J = 9.1, 2.9 Hz, 1H), 3.68 (s, 3H), 2.42 (s, 3H), 2.04-1.84 (m, 1H), 1.02 (dd, J = 6.2, 4.4 Hz, 2H), 0.94 (dd, J = 6.9, 4.4 Hz, 2H). | 444.3 |
| 15 | 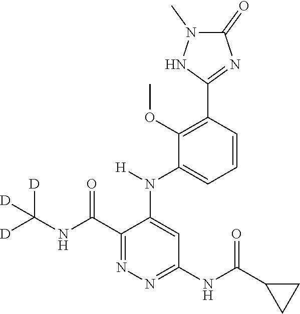 [SMILES: [2H]C([2H])([2H])NC(=O)c1nnc(NC(=O)C2CC2)cc1Nc1cccc(-c2nc(=O)n(C)[nH]2)c1OC] | 1H NMR (400 MHz, DMSO) δ 11.70 (s, 1H), 11.35 (s, 1H), 10.98 (s, 1H), 9.16 (s, 1H), 8.14 (s, 1H), 7.57 (d, J = 8.1 Hz, 1H), 7.48 (d, J = 7.7 Hz, 1H), 7.30 (t, J = 7.9 Hz, 1H), 3.66 (s, 3H), 3.38 (s, 3H), 2.08 (s, 1H), 0.82 (s, 4H). | 442.2 |

### Biological Example 1. TYK2 JH2 Domain Binding Assay

Assays were performed in 384-well plates with a final assay volume of 20 μL containing copper-polyvinyltoluene scintillation proximity assay (SPA) beads (PerkinElmer Life Sciences, catalogue no. RPNQ0095) at 80 g/ml, H3 probe (20 nM), the N-terminal His-tagged TYK2 pseudokinase domain (2.5 nM, purified by Pharmaron), and test compounds in assay buffer (50 mM HEPES, pH 7.5, 100 g ml-1 BSA, 5% DMSO). After incubating at room temperature for 30 min, the inhibition was calculated by the displacement of [3H] 3 binding as determined by scintillation counting. Dose-response curves were generated to determine the concentration required to inhibit H3 probe binding by 50% (IC50).

### Biological Example 2. IL-23 Induced STAT3 QUANTI-Blue in HEK-Blue™ IL-23 Cells

HEK-Blue™ IL-23 cells were prepared at 2×10⁵ cells/ml for adding 5000 cell/well and incubate plates containing compounds+cells for 1 hour at 37° C. Prepare IL-23 (treatment at final conc. 0.1 ng/mL) during incubation and add 2 ul per well of prepared cytokine in media to appropriate wells. The plate was incubated for overnight at 37° C. The next day, QUANTI-Blue solution was prepared. 18 ul of QUANTI-Blue solution per well was added into a new 384-well clear flat bottom plate, followed by addition of 2 ul of induced and treated HEK-Blue cell supernatant from overnight plate. The plate was incubated at 37° C. for 2 hours and SEAP levels determined.

Biological activity data for representative compounds of the invention (compound Examples 1-15 described hereinabove) tested according to the procedures described in Biological Examples 1 and 2 are provided in Table 2 below. A control compound, BMS-986165 (J. Med. Chem. 2019, 62, 20, 8973-8995), was also tested following Biological Examples 1 and 2. BMS986165 is believed to have the structure shown below:

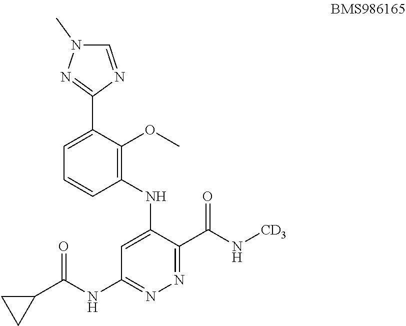 [SMILES: [2H]C([2H])([2H])NC(=O)c1nnc(NC(=O)C2CC2)cc1Nc1cccc(-c2ncn(C)n2)c1OC]

TABLE 2

| | TYK2 JH2 SPA | |
|---|---|---|
| | Binding assay | IL23 reporter |
| Compound | (IC50 nM) | assay_IC50 nM |
| Control (BMS-986165) | 5.9 | 3.76 |
| Example 1 | 26.99 | 318.28 |
| Example 2 | 5.56 | 15.66 |
| Example 3 | 16.08 | 6.5 |
| Example 4 | 35 | 18 |
| Example 5 | 39 | 6 |
| Example 6 | 30 | 18 |
| Example 9 | | 318 |
| Example 10 | | 74 |
| Example 11 | | 111 |
| Example 12 | | 15 |
| Example 13 | | 38 |
| Example 14 | | 71 |
| Example 15 | 89 | 25 |

### Biological Example 3. IL-12 Induced pSTAT4 in Human PBMC

Frozen Human PBMC are thawed and resuspended in complete media containing serum, then cells are diluted to 1.6×10⁶ cells/ml. 2.5 uL of compound or DMSO is added to the well at the desired concentrations and incubated at 1 hr at 37° C. 2.5 uL of stimulus (final concentration of 1.7 ng/mL IL-12) is added for 30 minutes prior to pSTAT4 analysis using cell lysates prepared and analyzed by AlphaLISA assay as per manufacturer protocol. The final DMSO concentration of compound in the assay is 0.1%.

### Biological Example 4. Selectivity Over Other JAK Kinases

This example compares selectivity profiles of exemplified compounds of the present disclosure (Compound Examples 4 and 5) and BMS986165. The results are shown in Table 3 below.

TABLE 3
Selectivity over other JAK kinases.

| | | IC50 (nM) | | |
|---|---|---|---|---|
| Assay type | Assay | BMS986165 | Example 4 | Example 5 |
| Biochemical | Tyk2 JH2 HTRF | 0.13 | 0.38 | 0.14 |
| assay | binding | | | |
| | Jak1 JH2 HTRF | 1.20 | 4.99 | 13.21 |
| | binding | | | |
| Cellular | IL-23 reporter assay | 3.5 | 18.3 | 6.1 |
| assay | (JAK2/Tyk2) | | | |
| | IFNα/β reporter | 1.3 | 3.7 | 4.6 |
| | assay (JAK1/Tyk2) | | | |
| | IL-2 reporter assay | 221.3 | 695.5 | >10000 |
| | (JAK1/3) | | | |
| | IFNγ reporter assay | 1087 | >10000 | >10000 |
| | (JAK1/2) | | | |

The following procedures were followed to test the activities of the selected compounds of the present disclosure and BMS9861615 in the assays referenced in Table 3 in this example.

Tyk2 and Jak1 JH2 HTRF binding were tested following procedures described in J Med Chem. 62(20):8973-8995 (2019).

IFN-alpha/beta reporter assay. Seed HEK-Blue™ IFN-a/b cells (1-3×10⁴ cells/well/100 ml) to 96 cell plate with DMEM (10% HFBS+1% PS) and incubate overnight at 5% CO₂&37° C. Diluent compounds in DMSO. Three times dilution and 10 doses. Add compounds to cell plate and incubate 1 h at 5% CO₂&37° C. Add IFN-alpha/beta (5 U/ml) to cell plate and incubate overnight at 5% CO₂&37° C. Prepare QUANTI-Blue Solution: add 100 μl of QB reagent and 100 μl of QB buffer to 9800 μl sterile water. Use cell supernatant and QUANTI-Blue solution in proportion of 1 to 10, incubate 2 h at 37° C. Read OD630.

IL-2 reporter assay. Seed HEK-Blue™ IL-2 Cells (1-3×10⁴ cells/well/100 ml) to 96 cell plate with DMEM (10% HFBS+1% PS) and incubate overnight at 5% CO₂&37° C. Diluent compounds in DMSO. Three times dilution and 10 doses. Add compounds to cell plate and incubate 1 h at 5% CO₂&37° C. Add IL-2 (1 ng/mL) to cell plate and incubate overnight at 5% CO₂&37° C. Prepare QUANTI-Blue Solution: add 100 μl of QB reagent and 100 μl of QB buffer to 9800 μl sterile water. Use cell supernatant and QUANTI-Blue solution in proportion of 1 to 10, incubate 2 h at 37° C. Read OD630.

IFN-γ reporter assay. Seed HEK-Dual™ IFN-γ Cells (1-3×10⁴ cells/well/100 ml) to 96 cell plate with DMEM (10% HFBS+1% PS) and incubate overnight at 5% CO₂&37° C. Diluent compounds in DMSO. Three times dilution and 10 doses. Add compounds to cell plate and incubate 1 h at 5% CO₂&37° C. Add IFN-γ (0.1 ng/mL) to cell plate and incubate overnight at 5% CO₂&37° C. Prepare QUANTI-Blue Solution: add 100 μl of QB reagent and 100 μl of QB buffer to 9800 μl sterile water. Use cell supernatant and QUANTI-Blue solution in proportion of 1 to 10, incubate 2 h at 37° C. Read OD630.

As can be seen from Table 3 above, selected compounds of the present disclosure were found to be more selective over other JAK kinases when compared to BMS986165, at least in the tested in vitro assays.

### Biological Example 5. In Vivo Inhibition of IL-12/IL-18 Induced IFN-Gamma

Selected exemplified compounds of the present disclosure (compound Examples 4 and 5) and BMS986165 were dosed orally to female C57BL/6J mice (Charles River). One hour later, 0.05 μg of recombinant rat IL-12 was administered IV. Another hour later following IL-12 administration, 1 μg of recombinant rat IL-18 was administered IV. Three hours later, blood was collected and serum were obtained by centrifugation for analysis of IFNγ by ELISA. Results show that representative tested compounds herein can dose-dependently inhibit IFNγ production. See FIGS. 1 (compound Example 4) and 2 (compound Example 5).

### Biological Example 6. In Vivo Mouse Psoriasis Model

Selected exemplified compounds of the present disclosure were also tested in a mouse psoriasis model induced by human IL-23. Psoriasis was induced in 6-8-week-old C57BL/6J female mice (Charles River) by intradermal injection of recombinant human IL-23 into the left ear. IL-23 injections were administered every other day from day 1 through day 9 of the study. Selected compounds at 7.5, 15, and 30 mg/kg, with 15 mg/kg BMS986165 as positive control were dosed BID by oral gavage, with the first dose given the evening before first IL-23 injection. Ear thickness was measured every other day, prior to the ear injection. The results show that representative tested compounds herein dose-dependently protect mice from IL-23-induced psoriasis.

### Biological Example 7. Dog PK

Selected exemplified compounds of the present disclosure were also tested in dog for pharmacokinetic studies. The Beagle dogs were fasted overnight with free access to drinking water prior to treatment. All the dosing solutions were freshly prepared prior to dose administration. In intravenous administration animal group were given a single dose of compound at 1 mg·kg-1. Blood for plasma samples were collected at pre-dose, 5, 15, 30 min, 1, 2, 4, 8, 12 and 24 h through vein into tubes anticoagulated with EDTA-K2. In intragastric administration animal group were given a single dose of compound at 5 mg·kg-1. Blood for plasma samples (˜0.5 mL) were collected at pre-dose, 15, 30 min, 1, 2, 4, 8, 12 and 24 h post-dose through vein into tubes anticoagulated with EDTA-K2. Blood samples were inverted several times and were held on wet ice pending centrifugation. The samples were centrifuged (within 20 minutes of collection) in a centrifuge set at 4° C. for 10 minutes at 1500-1600 g to obtain plasma. Plasma samples were transferred and frozen immediately after separation and stored in a freezer set at below −70° C. prior to analysis.

Selected PK results are shown in Table 4 below:

TABLE 4
Comparison of Dog PK profiles

| | |
|---|---|
| BMS986165 | Example 5 |

| | | | | |
|---|---|---|---|---|
| AUC₀₋ₜ (IV/PO) | 3.8/22.3 | uM · h | 6.4/47.5 | uM · h |
| Cmax (PO) | 2.7 | uM | 7.87 | uM |

The Summary and Abstract sections may set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 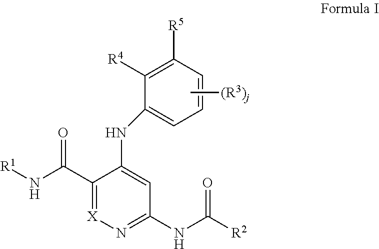
wherein:
X is CH or N;
R¹ is C₁₋₃ alkyl substituted by 0-7 deuterium atoms;
R² is optionally substituted C₁₋₆ alkyl, optionally substituted C₁₋₄ heteroalkyl, optionally substituted cycloalkyl (e.g., C₃₋₆ cycloalkyl), optionally substituted heterocyclyl (e.g., 4-8 membered heterocyclyl), or optionally substituted heteroaryl;
R³ at each occurrence is independently halogen, optionally substituted C₁₋₄ alkyl, or optionally substituted C₁₋₄ heteroalkyl;
j is 0, 1, 2, or 3;
R⁴ is C₁₋₆ alkyl optionally substituted with 1-3 R^{A}, S(O)ₚR^{B}, or OR^{C};
wherein:
p is 0, 1, or 2,
R^{A} at each occurrence is independently halogen, OH, C₁₋₆ alkyl optionally substituted with 1-3 R^{A1},
R^{B} is C₁₋₆ alkyl optionally substituted with 1-3 R^{A1},
R^{C} is hydrogen or C₁₋₆ alkyl optionally substituted with 1-3 R^{A2},
wherein R^{A1} at each occurrence is independently halogen, OH, or CN; R^{A2} at each occurrence is independently F or OH;
R⁵ is 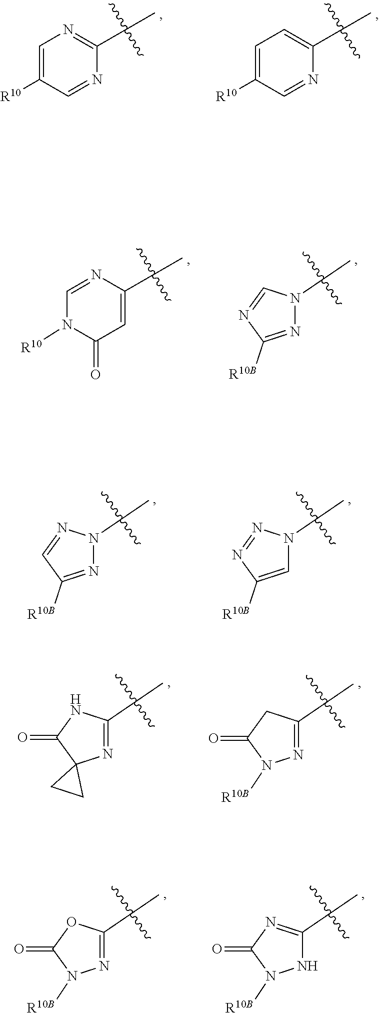
or -L¹-L²-Q-G, wherein:
R¹⁰ at each occurrence is independently an optionally substituted cycloalkyl (e.g., C₃₋₆ cycloalkyl), or optionally substituted heterocyclyl (e.g., 4-8 membered heterocyclyl);
R^{10B} at each occurrence is independently halogen, CN, an optionally substituted C₁₋₆ alkyl, an optionally substituted cycloalkyl (e.g., C₃₋₆ cycloalkyl), or optionally substituted heterocyclyl (e.g., 4-8 membered heterocyclyl);
L¹ is O, C═(O)NH, or null,
L² is C₁₋₄ alkylene, or null,
Q is an optionally substituted heterocycle or optionally substituted heteroaryl, and
G is CN, 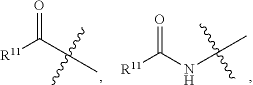
or a Michael acceptor,
wherein R¹¹ is hydrogen, an optionally substituted C₁₋₆ alkyl or an optionally substituted C₃₋₆ cycloalkyl.

2. The compound of claim 1, or a pharmaceutically acceptable salt thereof, wherein X is CH.

3. The compound of claim 1, or a pharmaceutically acceptable salt thereof, wherein X is N.

4. The compound of any one of claims 1-3, or a pharmaceutically acceptable salt thereof, wherein R¹ is CH₃, C₂H₅, CD₃, or CD₂CD₃.

5. The compound of any one of claims 1-4, or a pharmaceutically acceptable salt thereof, wherein R² is a 3-6 membered cycloalkyl, such as cyclopropyl, cyclobutyl, which is optionally substituted with 1-4 substituents independently selected from CN, halogen (e.g., F), OH, and optionally substituted C₁₋₆ alkyl.

6. The compound of any one of claims 1-4, or a pharmaceutically acceptable salt thereof, wherein R² is cyclopropyl optionally substituted with 1-4 substituents independently selected from CN, halogen, OH, and optionally substituted C₁₋₆ alkyl.

7. The compound of any one of claims 1-4, or a pharmaceutically acceptable salt thereof, wherein R² is cyclopropyl.

8. The compound of any one of claims 1-7, or a pharmaceutically acceptable salt thereof, wherein j is 1.

9. The compound of any one of claims 1-8, or a pharmaceutically acceptable salt thereof, wherein when present, R³ at each occurrence is independently F, Cl, or C₁₋₄ alkyl optionally substituted with F.

10. The compound of any one of claims 1-7, or a pharmaceutically acceptable salt thereof, wherein j is 0.

11. The compound of any one of claims 1-10, or a pharmaceutically acceptable salt thereof, wherein R⁴ is OMe.

12. The compound of any one of claims 1-11, or a pharmaceutically acceptable salt thereof,
wherein R⁵ is 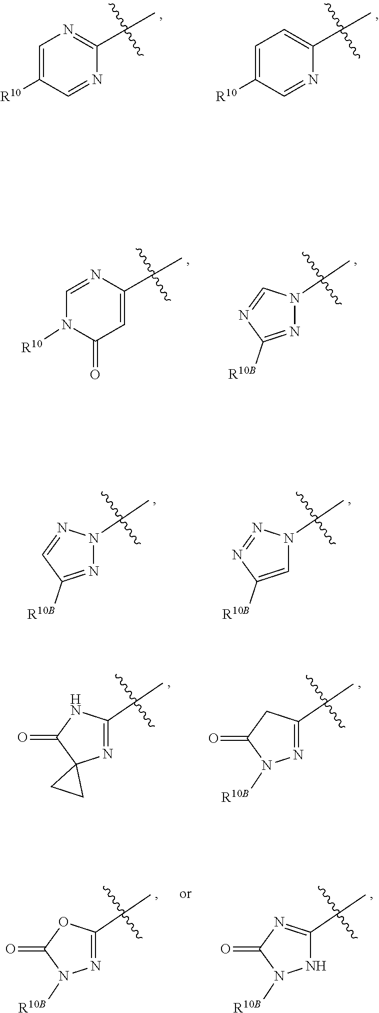
wherein R¹⁰ is an optionally substituted C₃₋₆ cycloalkyl, R^{10B} is an optionally substituted C₁₋₄ alkyl or an optionally substituted C₃₋₆ cycloalkyl.

13. The compound of claim 12, or a pharmaceutically acceptable salt thereof, wherein R¹⁰ is 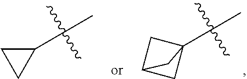
R^{10B} is methyl, CF₃, or cyclopropyl.

14. The compound of any one of claims 1-11, or a pharmaceutically acceptable salt thereof, wherein R⁵ is -L¹-L²-Q-G.

15. The compound of claim 14, or a pharmaceutically acceptable salt thereof, wherein L¹ is O.

16. The compound of claim 14, or a pharmaceutically acceptable salt thereof, wherein L¹ is null.

17. The compound of claim 14, or a pharmaceutically acceptable salt thereof, wherein L¹ is C═(O)NH:

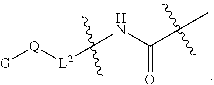

18. The compound of any one of claims 14-17, or a pharmaceutically acceptable salt thereof, wherein L² is null.

19. The compound of any one of claims 14-17, or a pharmaceutically acceptable salt thereof, wherein L² is C₁₋₄ alkylene, e.g., CH₂.

20. The compound of any one of claims 14-19, or a pharmaceutically acceptable salt thereof, wherein Q is a 4-7 membered monocyclic heterocyclic ring having 1 or 2 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

21. The compound of any one of claims 14-19, or a pharmaceutically acceptable salt thereof, wherein Q is a 4-7 membered monocyclic heterocyclic ring selected from:

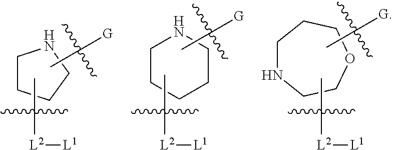

22. The compound of any one of claims 14-19, or a pharmaceutically acceptable salt thereof, wherein Q is a 4-7 membered monocyclic heterocyclic ring selected from:

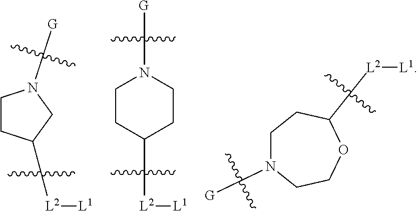

23. The compound of any one of claims 14-19, or a pharmaceutically acceptable salt thereof,
wherein Q is a 6-12 membered bicyclic heterocyclic ring having 1-4 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F,
wherein the bicyclic heterocyclic ring is a fused, spiro, or bridged bicyclic ring, wherein one of the rings is optionally aromatic or heteroaromatic.

24. The compound of any one of claims 14-19, or a pharmaceutically acceptable salt thereof, wherein Q is a 6-10 membered bicyclic heterocyclic ring selected from: 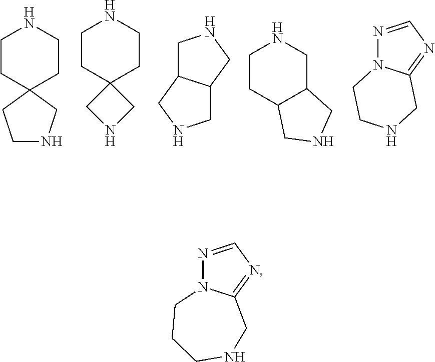
each of which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

25. The compound of any one of claims 14-19, or a pharmaceutically acceptable salt thereof, wherein Q is a 6-10 membered bicyclic heterocyclic ring selected from:

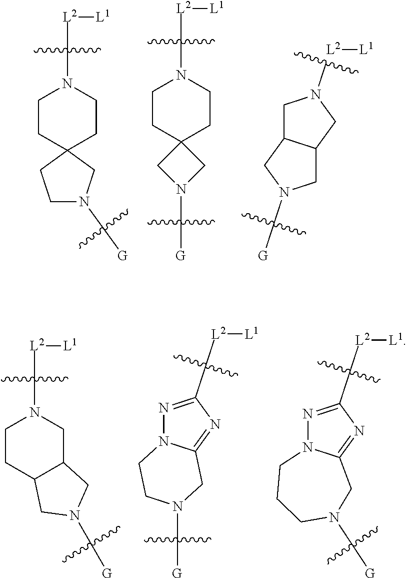

26. The compound of any one of claims 14-19, or a pharmaceutically acceptable salt thereof, wherein Q is a 5 or 6 membered heteroaryl having 1-4 ring heteroatoms independently selected from N, O, and S, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

27. The compound of any one of claims 14-19, or a pharmaceutically acceptable salt thereof, wherein Q is pyridine or pyrimidine, which is optionally substituted with one or more (e.g., 1, 2, or 3) R^{s1},
wherein R^{s1} at each occurrence is independently F, Cl, CN, OH, oxo (as valency permits), C₁₋₄ alkyl optionally substituted with F, cyclopropyl, cyclobutyl, or C₁₋₄ alkoxy optionally substituted with F.

28. The compound of any one of claims 14-19, or a pharmaceutically acceptable salt thereof, wherein Q is

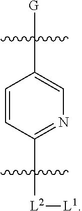

29. The compound of any one of claims 14-28, or a pharmaceutically acceptable salt thereof, wherein G is CN.

30. The compound of any one of claims 14-28, or a pharmaceutically acceptable salt thereof, wherein G is 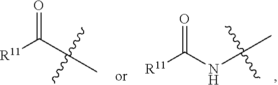
wherein R¹¹ is hydrogen or C₁₋₄ alkyl, such as methyl, optionally substituted with halogen.

31. The compound of any one of claims 14-28, or a pharmaceutically acceptable salt thereof, wherein G is a Michael acceptor.

32. The compound of any one of claims 14-28, or a pharmaceutically acceptable salt thereof, wherein G is

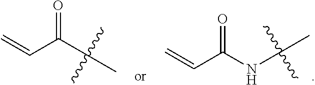

33. The compound of any one of claims 1-11, or a pharmaceutically acceptable salt thereof, wherein R⁵ is selected from:

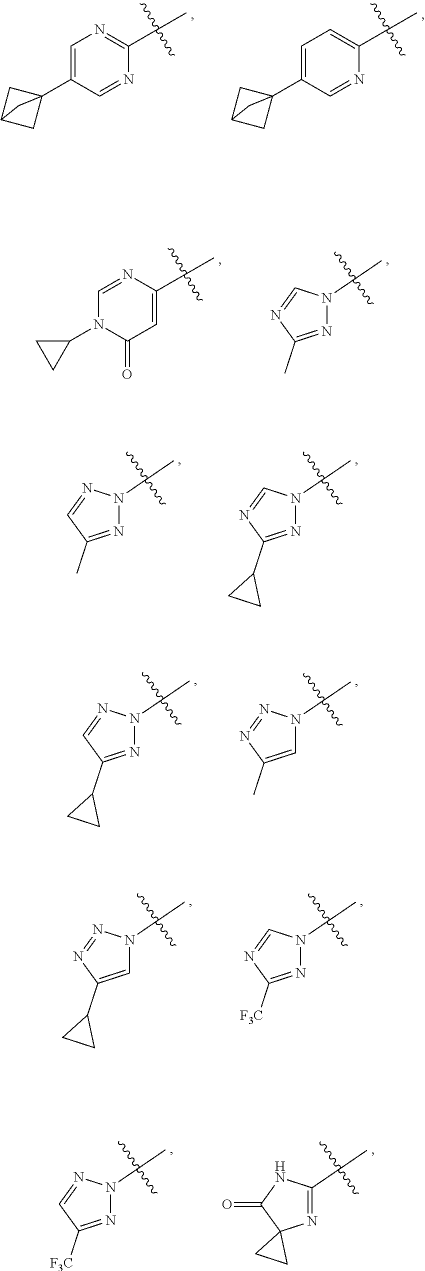

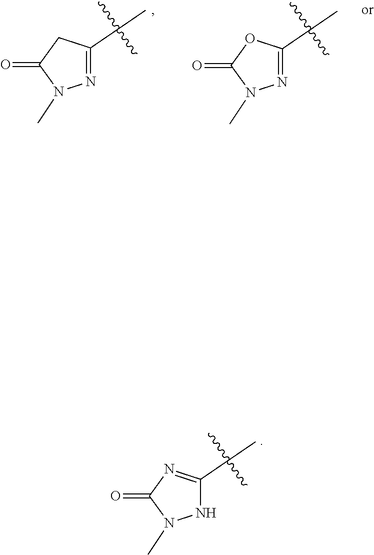

34. The compound of any one of claims 1-11, or a pharmaceutically acceptable salt thereof, wherein R⁵ is selected from:

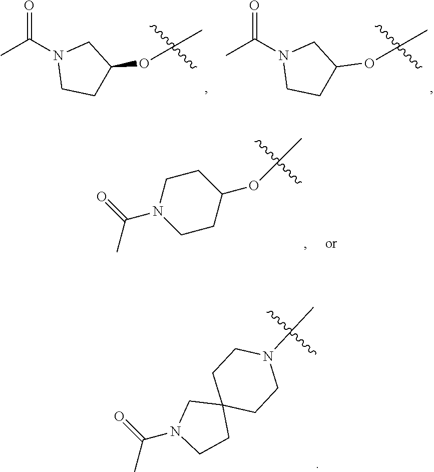

35. The compound of any one of claims 1-11, or a pharmaceutically acceptable salt thereof, wherein R⁵ is selected from:

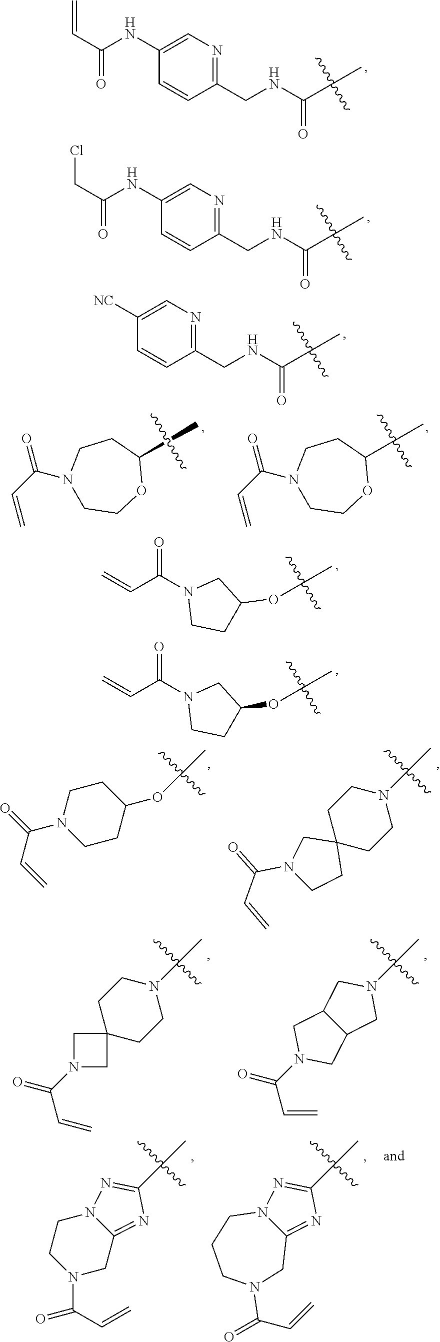

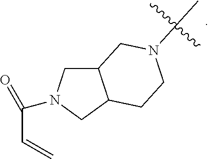 [SMILES: *N1CCC2CN(C(=O)C=C)CC2C1]

36. A compound selected from the compounds in Table 1 herein, or compounds of Examples 1-15, or a pharmaceutically acceptable salt thereof.

37. A pharmaceutical composition comprising the compound of any one of claims 1-36, or a pharmaceutically acceptable salt thereof, and a pharmaceutically acceptable excipient.

38. A method of inhibiting TYK2 in a subject or biological sample comprising contacting the subject or biological sample with an effective amount of the compound of any one of claims 1-36, or a pharmaceutically acceptable salt thereof, or the pharmaceutical composition of claim 37.

39. A method of treating a TYK2-mediated disease or disorder in a subject in need thereof, the method comprising administering to the subject a therapeutically effective amount of the compound of any one of claims 1-36, or a pharmaceutically acceptable salt thereof, or the pharmaceutical composition of claim 37.

40. The method of claim 39, wherein the TYK2-mediated disease or disorder is an autoimmune disease or disorder, an inflammatory disease or disorder, a proliferative disease or disorder, an endocrine disease or disorder (e.g., polycystic ovary syndrome, Crouzon's syndrome, or type 1 diabetes), a neurological disease or disorder (e.g., Alzheimer's disease), and/or a disease or disorder associated with transplantation (e.g., transplant rejection or graft versus host disease).

41. The method of claim 39, wherein the TYK2-mediated disease or disorder is an autoimmune disease or disorder selected from type 1 diabetes, ankylosing spondylitis, cutaneous lupus erythematosus, systemic lupus erythematosus, multiple sclerosis, systemic sclerosis, psoriasis, Behçet's disease, POEMS syndrome, Crohn's disease, ulcerative colitis, inflammatory bowel disease, and combinations thereof.

42. The method of claim 39, wherein the TYK2-mediated disease or disorder is an inflammatory disease or disorder selected from rheumatoid arthritis, asthma, chronic obstructive pulmonary disease, psoriasis, hepatomegaly, Crohn's disease, ulcerative colitis, inflammatory bowel disease, and combinations thereof.

43. The method of claim 39, wherein the TYK2-mediated disease or disorder is a proliferative disease or disorder, such as a hematological cancer (e.g., leukemia, such as T-cell leukemia, e.g., T-cell acute lymphoblastic leukemia (T-ALL)).

44. The method of claim 39, wherein the TYK2-mediated disease or disorder is associated with type I interferon, IL-10, IL-12, and/or IL-23 signaling.

45. A method of treating psoriasis in a subject in need thereof, the method comprising administering to the subject a therapeutically effective amount of the compound of any one of claims 1-36, or a pharmaceutically acceptable salt thereof, or the pharmaceutical composition of claim 37.

46. A method of treating psoriatic arthritis in a subject in need thereof, the method comprising administering to the subject a therapeutically effective amount of the compound of any one of claims 1-36, or a pharmaceutically acceptable salt thereof, or the pharmaceutical composition of claim 37.

47. A method of treating systemic lupus erythematosus in a subject in need thereof, the method comprising administering to the subject a therapeutically effective amount of the compound of any one of claims 1-36, or a pharmaceutically acceptable salt thereof, or the pharmaceutical composition of claim 37.

48. A method of treating Crohn's disease in a subject in need thereof, the method comprising administering to the subject a therapeutically effective amount of the compound of any one of claims 1-36, or a pharmaceutically acceptable salt thereof, or the pharmaceutical composition of claim 37.

49. A method of treating ulcerative colitis in a subject in need thereof, the method comprising administering to the subject a therapeutically effective amount of the compound of any one of claims 1-36, or a pharmaceutically acceptable salt thereof, or the pharmaceutical composition of claim 37.

50. A method of treating inflammatory bowel disease in a subject in need thereof, the method comprising administering to the subject a therapeutically effective amount of the compound of any one of claims 1-36, or a pharmaceutically acceptable salt thereof, or the pharmaceutical composition of claim 37.
